(19) United States
(12) Patent Application Publication
(10) Pub. No.: US 2025/0051348 A1
(43) Pub. Date: Feb. 13, 2025

(54) MECHANOPHORIC MATRICES AND METHODS OF PREPARING SAME

(71) Applicant: Technion Research & Development Foundation Limited, Haifa (IL)

(72) Inventors: Joshua M. GROLMAN, Haifa (IL); Yifan LIAO, Haifa (IL); Hang ZHANG, Haifa (IL); Charles Eliezer DIESENDRUCK, Haifa (IL)

(73) Assignee: Technion Research & Development Foundation Limited, Haifa (IL)

(21) Appl. No.: 18/803,753

(22) Filed: Aug. 13, 2024

Related U.S. Application Data
(60) Provisional application No. 63/532,401, filed on Aug. 13, 2023.

(51) Int. Cl. C07D 491/107

(52) U.S. Cl. CPC C07D 491/107

(57) ABSTRACT

Mechanophoric compounds featuring one or more, or two or more, clickable group(s), which are capable of being coupled to naturally occurring, synthetic and/or metallic substrates via Click chemistry, method of preparing mechanophoric matrices using the mechanophoric compounds, and mechanophoric matrices obtainable thereby, and uses thereof for detecting force-induced damage in the matrices by a colorimetric change are provided.

### RELATED APPLICATION(S)

This application claims the benefit of priority under 35 USC § 119(e) of U.S. Provisional Patent Application No. 63/532,401 filed on Aug. 13, 2023, the contents of which are all incorporated by reference as if fully set forth herein in their entirety.

### BACKGROUND OF THE INVENTION

The present invention, in some embodiments thereof, relates to material science, and, more particularly, but not exclusively, to a novel methodology for converting varying matrices into mechanophores, to novel mechanophoric compounds and novel matrices obtained thereby and to applications thereof.

Mechanophores are stimuli-responsive materials that change color or become fluorescent upon application of a mechanical stimulus, typically by undergoing a chemical reaction when a load is applied to a matrix containing same, and are also referred to as mechanically-responsive materials. As such, mechanophores can serve as stress or force sensors in a myriad of applications, for example, by incorporating mechanophoric-containing matrices into articles or systems and thereby allow determining the real-time stress distribution in these articles or systems directly through a visual response.

Mechanophoric materials typically incorporate force-sensitive molecules, also known and referred to herein as “mechanophoric compounds”.

A wide array of applications that require continuous structural health monitoring can benefit from mechanophoric sensing. These include, for example, detecting material damage [Vidavsky et al. J. Am. Chem. Soc. 2019, 141 (25), 10060-10067; Patrick et al. Nature 2016, 540 (7633), 363-370], flexible electronics, protective coatings, polymer matrix composites, engineered plastics such as self-healing plastics [Diesendruck et al. In Self-Healing Polymers. Binder, W. H., Eds.; Wiley, 2013; pp. 193214], and drug delivery [Versaw et al. J. Am. Chem. Soc. 2021, 143 (51), 21461-21473].

Molecular force sensors have been investigated also in biological-derived materials, particularly in the field of mechano-biology in the context of the links between development, disease, and mechanics. For example, the importance of cells sensing their environment [Grolman et al. Proc. Natl. Acad. Sci. U.S.A 2020, 117 (42), 25999-26007; Darnell et al. Proc. Natl. Acad. Sci. U.S.A 2018, 115 (36), No. E8368-E8377] and directing disease progression such as in myelofibrosis [Vining et al. Nat. Mater. 2022, 21 (8), 939-950] has been shown.

However, the challenges of molecular force sensor incorporation, which would allow connecting between mechanical and biochemical processes, remain unsolved.

Widespread use of force-sensitive molecules is impeded by two main factors: (i) the difficulty in their integration into different bulk materials [Zhang et al. Macromolecules 2014, 47 (19), 6783-6790; Chen et al. “Nat. Chem. 2012, 4 (7), 559-562 and (ii) low sensitivity, which requires very large strains for activation.

The difficulty of integrating a mechanophoric compound (force-sensitive molecule) into the bulk material (typically a polymeric substance or matrix) is further magnified because controlling the placement of the mechanophore is usually difficult, with the exception of heterointerfaces. Typically, the mechanophores are limited by their placement on either a statistical center of the matrix, which is usually the most sensitive to strain, or close to the ends. Because mechanophores are generally introduced into synthetic polymer chains during the chemical synthesis (as initiators, co-monomers, or cross-linkers, typically using highly controlled laboratory scale conditions) prior to polymer processing (thermoplastics) or during its formation (thermosets), the precision of placement is not well defined. This also precludes many types of polymers from use, particularly glaring are naturally-derived ones. In addition, as mechanophores are introduced in a chemical step which is mostly unavailable in the thermoplastic industry, which typically restricts itself to thermal processes that can be detrimental to these sensitive compounds [Kiebala et al. Adv. Funct. Mater. 2023, 33 (50), 2304938]. This limitation is even more pronounced in biologically-derived materials, where in vivo or in vitro chemical synthesis makes pre-polymerization introduction of mechanophores difficult. When incorporated during the chemical step (e.g., polymerization), such mechanophores still need to undergo processing, such as thermal molding in thermoplastics, which occurs under fairly aggressive conditions that can be detrimental to most mechanophores (FIG. 16A) [Willis-Fox et al. Trends Chem. 2023, 5 (6), 415-431].

Further, integration of mechanophores often involves grafting photo-initiators to both ends, which can be expensive and restrict the choice of polymers to synthetic ones. In the case of, for example, spiropyran, it may also limit the polymer processing of the mechanophore as spiropyrans have certain stability issues and are not very inert, which necessitates its performance under harsh reaction conditions and/or an inert atmosphere.

In-situ conjugation of mechanophores to polymeric matrices further faces certain obstacles such as limited solubility, low reactivity, and/or extreme reaction conditions.

The lack of sensitivity of many mechanophores currently in use is also of concern because it hinders the use of these molecular-scale sensors in commercial applications. Typically, common mechanophores (e.g., spiropyran) require more than 100% strain to achieve a detectable output. This can be an obstacle if the desired application is, e.g., drug delivery, because it necessitates the use of ultra-sonication to provide sufficient strain to deliver the payload, which might injure adjacent tissues. Also, many potential commercial applications of mechanophores in engineered plastics require sensitivity below 10% strain, which is the standard by which most materials behave linearly.

Thus, although the ability to sense and report mechanical forces and damage by, e.g., colorimetric change, is an under-appreciated feature of many systems, which can be beneficially applied in many commercial applications and products (e.g., consumer goods, aircraft components), the challenges associated with the integration of mechanophores in the bulk matrix limit their use.

Exemplary force-sensitive molecules that are currently practiced include, e.g., spirolactams, spiropyrans, dianthracenes, merocyanines, naphthopyrans, diarylbibenzothiophenonyl (DABBT), diarylbibenzofuranone (DABBF), tetraarylsuccinonitrile (TASN), bis[3H]-naphtho[2,1-b]pyran thiophenes (BNPTs), bis-naphthopyran (BPT) CDeneke et al. Soft Matter, 2020,16, 6230-6252; Kim et al. Polym. Chem., 2022, 13, 5177-5187].

Table 1 below summarizes the currently known methods for functionalizing polymeric matrices with spiropyrans.

TABLE 1

| | | | | Special | Purification |
|---|---|---|---|---|---|
| Reference | Catalyst | Solvent | Gas | treatment | process |
| Wang et al., “Enhanced | Cu(0) | DMSO | Argon | Three | Precipitation |
| Mechanophore Activation | CuBr₂ | | | freeze- | in 1:1 |
| within Micelles”, | | | | pump-thaw | CH₃OH:H2O |
| Macromolecules, 2016, | | | | cycles | |
| 49(1), 98-104 | | | | | |
| Zhang et al. | Dibutyltin | THF | N₂ | Three | — |
| “Mechanoresponsive | dilaurate | | | freeze- | |
| PSPnBA-PS Triblock | | | | pump-thaw | |
| Copolymers via | | | | cycles | |
| Covalently Embedding | | | | | |
| Mechanophore”, ACS | | | | | |
| Macro Lett. 2013, 2(8), | | | | | |
| 705-709 | | | | | |
| Jiang et al., | Stannous | Anhydrous | — | 110° C., 24 | Precipitated |
| “Mechanoresponsive PS- | octoate | toluene | | hours | in methanol |
| PnBA-PS Triblock | — | THF | N₂ | 60° C., 2 | — |
| Copolymers via | | | | days | |
| Covalently Embedding | Dicumyl | Toluene | — | 100° C., 30 | — |
| Mechanophore”, ACS | peroxide | | | minutes | |
| Macro Lett., 2013, 2, | | | | | |
| 705-709 | | | | | |
| Potisek et al., | Cu(0) | DMSO | Argon | Three | Filtration, |
| “Mechanophore-linked | Me₆TREN | | | freeze- | Precipitated |
| addition polymers”, J. | | | | pump-thaw | in methanol |
| Am. Chem. Soc. 2007, | | | | cycles | |
| 129, 45, 13808-13809 | | | | | |
| Lee et al. “Exploiting | — | THF | N₂ | 70° C. for 2 | Degassed, |
| Force Sensitive | | | | days | Removed |
| Spiropyrans as Molecular | | | | | solvent under |
| Level Probes”, | | | | | high vacuum |
| Macromol. 2013, 46(10), | | | | | |
| 3746-3752 | | | | | |
| Kim et al. “Enhancement | Sylgard | xylene | — | 70° C. for 4 | — |
| of Mechano-Sensitivity | 184 curing | | | hours; | |
| for Spiropyran-Linked | agent | | | followed by | |
| Poly(Dimethylsiloxane) | | | | 25° C. for | |
| via Solvent Swelling”, | | | | 12 hours | |
| Macromol. 2020, 53(18), | | | | | |
| 7954-7961 | | | | | |
| Davis et al., “Force- | Cu(0) | DMSO | Argon | Three | Filtration, |
| induced activation of | Me₆TREN | | | freeze- | Precipitated |
| covalent bonds in | | | | pump-thaw | in methanol |
| mechanoresponsive | | | | cycles | |
| polymeric materials”, | Benzoyl | Water | Argon | N,N- | — |
| Nature, 2009, 459, 68-72 | peroxide | | | dimethylaniline | |
| Weng et al. “Spiropyran | Dibutyltin | Tetrahydro | N₂ | Three | — |
| as a Mechanochromic | dilaurate | furan | | freeze- | |
| Probe in Dual Cross- | | (THF) | | pump-thaw | |
| Linked Elastomers”, | | | | cycles | |
| Macromolecules 2014, 47 | | | | | |
| (19), 6783-6790, 2016, 49, | | | | | |
| 98 | | | | | |

U.S. Pat. No. 8,236,914 discloses a mechanochromic material that includes a polymer having a backbone containing a mechanophore.
U.S. Pat. No. 11,174,359 discloses a composite material comprising a matrix material, a fiber dispersed in the matrix material, and a UV-light sensitive mechanophore grafted to the surface of the fiber.
U.S. Pat. No. 11,034,791 discloses a composition comprising a cinnamide-derived mechanophore which is covalently-bound and embedded in a thermosetting polymer network.
U.S. Pat. No. 10,908,077 discloses methods and systems for detecting damage in a mechanophore-embedded composite material.
U.S. Patent Application Publication No. 2013/0269445 discloses coating compositions having a polymeric network that incorporates a plurality of chain-centered polyurethane mechanophores.
Li et al. [Polymer, 2017, 112(10), 219-227] describes covalent functionalization of vinyl polymers such as EVA by acrylate-functionalized spiropyran mechanophoric cross-linker, in the presence of a catalyst.
Additional Background Art includes Liao et al., J. Am. Chem. Soc. 2024, 146, 17878-17886; Battigelli et al. Bioconjugate Chem. 2022, 33 (2), 263-271; Liu et al. Chem. Soc. Rev. 2017, 46 (16), 5147-5172; and Watabe et al. Macromolecules 2021, 54, 4, 1725-1731; Li et al. Am. Chem. Soc. 2014, 136, 45, 15925-15928; Grossweiler et al. J. Am. Chem. Soc. 2015, 137, 19, 6148-6151; and Barbee et al. J. Am. Chem. Soc. 2018, 140 (40), 12746-12750.

### SUMMARY OF THE INVENTION

According to an aspect of some embodiments of the present invention there is provided a method of preparing a mechanophoric matrix (a mechanophoric composition-of-matter), the method comprising contacting a substrate (e.g., a polymeric material, a metallic material or substrate) that features a plurality of a first reactive group with a mechanophoric compound that features at least one of a second reactive group, wherein the first and second reactive groups are capable of undergoing a chemical reaction that leads to a covalent bond formation therebetween, thereby preparing the mechanophoric matrix.

According to some of any of the embodiments described herein, the contacting is performed under conditions that allow the chemical reaction to occur.

According to some of any of the embodiments described herein, the contacting is performed in the absence of a catalyst.

According to some of any of the embodiments described herein, the contacting is performed at a temperature lower than 100, or lower than 80, or lower than 60, or lower than 40, ° C.

According to some of any of the embodiments described herein, the contacting comprises contacting the substrate (e.g., a polymeric material, a metallic substrate) with a solution that comprises the mechanophoric compound.

According to some of any of the embodiments described herein, the solution comprises water and/or a polar organic solvent, such as for example, an alcohol (e.g., ethanol), and/or dichloromethane.

According to some of any of the embodiments described herein, the chemical reaction is a quantitative reaction.

According to some of any of the embodiments described herein, the chemical reaction is a Click reaction, as described and defined herein in any of the respective embodiments. According to some of these embodiments, the first reactive group is a first clickable group and the second reactive group is a second clickable group (which is complementary with the first clickable group), such that the first and second reactive or clickable groups are capable of undergoing a Click reaction with one another to thereby form a covalent bond (a Click bond) therebetween, thereby preparing the mechanophoric matrix or composition-of-matter.

According to some of any of the embodiments described herein, the Click reaction features at least one, or all, of the following: it is performed in the absence of a catalyst; it is performed at a temperature lower than 100, or lower than 80, or lower than 60, or lower than 40, ° C.; it is a quantitative reaction; and it is performed at ambient (e.g., oxygen-containing) environment.

According to some embodiments of any of the embodiments described herein, the method comprises contacting a substrate that features at least one, or a plurality, of the first reactive group (a first clickable group) with a mechanophoric compound that features at least one of a second reactive group (a second clickable group), wherein each of the first and second reactive groups are complementary clickable groups that are capable of undergoing a Click reaction with one another to thereby form a covalent bond (a Click bond) therebetween, as described herein in any of the respective embodiments, thereby preparing the mechanophoric matrix or composition-of-matter.

According to some embodiments of any of the embodiments described herein, the Click reaction is selected from Huisgen 1,3-dipolar cycloaddition, a thiol-ene reaction, a Diels-Alder reaction, an inverse electron demand Diels-Alder reaction, an isonitrile-tetrazine cycloaddition, a nucleophilic substitution (e.g., between small strained cycloalkyl or heteroalicyclic and alkyne), an amide bond formation (e.g., between carbonyl, carboxylate, C-amide and amine), strain-promoted alkyne-nitrogen cycloaddition (SPANC), nucleophilic ring opening of strained heteroalicyclic (e.g., aziridine, epoxide), and strain-promoted azide-alkyne cycloaddition (SPAAC). According to some of these embodiments, the first and second reactive or clickable groups are selected suitable for participating in a Click reaction as selected from the aforementioned reactions.

According to some embodiments of any of the embodiments described herein, the first reactive/clickable group is not an acrylic group (e.g., an acrylate, a methacrylate, an acrylamide or a methacrylamide group) or does not comprise an acrylic group.

According to some embodiments of any of the embodiments described herein, the mechanophoric compound is devoid of an acrylic group.

According to some embodiments, the mechanophoric compound can comprise more than one type of a reactive/clickable group, or two or more mechanophoric compounds are contacted with the substrate and each features a different first reactive/clickable group and/or the substrate can feature more than one type of a second reactive/clickable group, such that two or more types of covalent (Click) bonds are formed upon contacting the mechanophoric compound and the substrate.

According to some embodiments of any of the embodiments described herein, the second reactive (clickable) group is or comprises an alkene.

According to some embodiments of any of the embodiments described herein, the second reactive (clickable) group is or comprises a strained cycloalkyl or a strained unsaturated cycloalkyl.

According to some embodiments of any of the embodiments described herein, the second reactive (clickable) group is or comprises a norbornene.

According to some embodiments of any of the embodiments described herein, the first reactive (clickable) group is or comprises an azide.

According to some embodiments of any of the embodiments described herein, the second reactive group is or comprises a strained unsaturated cycloalkyl, e.g., norbornene, and the first reactive group is or comprises an azide.

According to some embodiments of any of the embodiments described herein, the first reactive (clickable) group is or comprises a tetrazine.

According to some embodiments of any of the embodiments described herein, the second reactive group is or comprises a strained unsaturated cycloalkyl, e.g., norbornene, and the first reactive (clickable) group is or comprises a tetrazine.

According to some embodiments of any of the embodiments described herein, the first reactive (clickable) group is or comprises a thiol.

According to some embodiments of any of the embodiments described herein, the second reactive group is or comprises a strained unsaturated cycloalkyl, e.g., norbornene, and the first reactive (clickable) group is or comprises a thiol.

According to some embodiments of any of the embodiments described herein, the substrate is or comprises a polymeric matrix, which is also referred to herein throughout as a polymeric material.

According to some embodiments of any of the embodiments described herein, the polymeric material comprises a natural polymeric substance and/or a synthetic polymeric substance.

According to some embodiments of any of the embodiments described herein, the synthetic polymeric substance is selected from an epoxy resin, a polystyrene, a polyolefin, a polyester, a polyamine (e.g., nylon), a polyurethane, an acrylic polymer, a polyvinyl polymer, a rubber, a silicone, a co-polymer or block-co-polymer comprising two or more of the foregoing, and any combination thereof.

According to some embodiments of any of the embodiments described herein, the synthetic polymeric substance is or comprises a polystyrene (e.g., a styrene-vinyl benzyl chloride copolymer). According to some of these embodiments, the first reactive group is or comprises an azide.

According to some embodiments of any of the embodiments described herein, the synthetic polymeric substance is or comprises an epoxy resin. According to some of these embodiments, the first reactive group is or comprises an epoxide (oxirane).

According to some embodiments of any of the embodiments described herein, the natural polymeric substance is selected from a polysaccharide (e.g., alginate, cellulose, chitin), and a proteinaceous substance.

According to some embodiments of any of the embodiments described herein, the natural polymeric substance is or comprises an alginate. According to some of these embodiments, the first reactive group is or comprises a tetrazine.

According to some embodiments of any of the embodiments described herein, the natural polymeric substance is a fibrous proteinaceous substance (e.g., wool, silk).

According to some embodiments of any of the embodiments described herein, the natural polymeric substance is wool. According to some of these embodiments, the first reactive group is or comprises a thiol.

According to some embodiments of any of the embodiments described herein, the substrate is a metallic substance, that is, it comprises in at least a portion thereof a metal, which can be arranged, for example, as a metal film.

According to some embodiments of any of the embodiments described herein, the substrate comprises a metallic material, for example, a metal film such as a gold film.

According to some of these embodiments, the metallic substrate comprises a metal (e.g., stainless steel) having a metallic film deposited on at least a portion thereof. According to some of these embodiments, the metallic film is a gold film that features at least one, preferably a plurality of the first reactive (clickable) group (e.g., thiol group(s)).

According to some embodiments of any of the embodiments described herein, the metallic substrate comprises gold. According to some of these embodiments, the gold features at least one, preferably a plurality of the first reactive (clickable) group (e.g., thiol group(s)).

According to some embodiments of any of the embodiments described herein, the method further comprises generating the first reactive (clickable) group or a plurality of such groups in and/or on the substrate (e.g., polymeric material or matrix, metallic substance), preferably on at least a portion of the surface of the substrate.

According to some embodiments of any of the embodiments described herein, the mechanophoric compound features at least two of the second reactive (clickable) group.

According to some embodiments of any of the embodiments described herein, the mechanophoric compound is represented by Formula I:

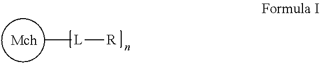

wherein:
Mch is a force-responsive or force-sensitive moiety (derived from a force-sensitive molecule or force-response compound);
L is a linking group or absent;
R is the second reactive (clickable) group; and
n is a positive integer, or an integer greater than 1, representing the number of second reactive/clickable groups in the mechanophoric compound.

According to some embodiments of any of the embodiments described herein, Mch is selected from a spiropyran (SP) moiety, a dithienylethene moiety, a spirolactam moiety, a dianthracene moiety, a merocyanine (MC) moiety, a naphthopyran (NP) moiety, a bis[3H]-naphtho[2,1-b]pyran thiophene moiety, a bis-naphthopyran moiety, a copper complex, a disulfide moiety, a diarylbibenzothiophenonyl (DABBT) moiety, a diarylbibenzofuranone (DABBF) moiety, a tetraarylsuccinonitrile (TASN) moiety, a ladderene moiety, an oxazime moiety, a fulminic acid moiety, each being optionally substituted by one or more substituents other than the [L-R].

According to some embodiments of any of the embodiments described herein, Mch is or comprises a spiropyran moiety.

According to some embodiments of any of the embodiments described herein, the L linking group is selected from an alkylene, an alkenylene, an alkynylene, a cycloalkyl linking group, an aryl linking group, a heteroaryl linking group, a heteroalicyclic linking group, —O—, —S—, an alkoxylene linking group, an aryloxylene linking group, a thioalkoxylene linking group, a thioaryloxylene linking group, a sulfinyl linking group, a sulfonyl linking group, a sulfonate linking group, a sulfate linking group, a cyano linking group, a phosphonyl linking group, a phosphinyl linking group, a carbonyl linking group, a thiocarbonyl linking group, an urea linking group, a thiourea linking group, a carbamyl linking group, a thiocarbamyl linking group, an amido linking group, a carboxy linking group, a sulfonamido linking group, a guanyl linking group, a guanidinyl linking group, a hydrazine linking group, a hydrazide linking group, a thiohydrazide linking group, and an amine linking group, as these groups are described and defined herein.

According to some embodiments of any of the embodiments described herein, L is or comprises a carboxy linking group (—C(═O)—O—or —O—C(═O)—).

According to some embodiments of any of the embodiments described herein, n is an integer greater than 1 (e.g., is 2, 3, 4 or higher).

According to some of these embodiments, when n is greater than 1, each of the —L—R moieties are the same. According to some of these embodiments, at least two of the —L—R moieties differ from one another by the linking group and/or the second reactive/clickable group.

According to some embodiments of any of the embodiments described herein, n is 2.

According to some embodiments of any of the embodiments described herein, the mechanophoric compound is represented by Formula II:

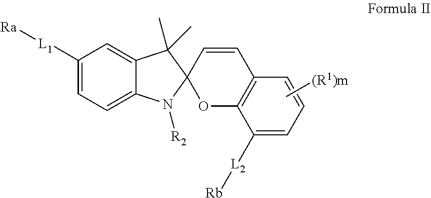

wherein:
Ra and Rb are each independently selected from a substituent, as described herein, and the second reactive (clickable) group, as described herein in any of the respective embodiments and any combination thereof, or is absent, wherein at least one of Ra and Rb is the second reactive (clickable) group as described herein;
L₁ and L₂ are each independently the linking group, as described herein in any of the respective embodiments and any combination thereof, or is absent;
R¹ is a substituent selected from alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, carbamate, thiocarbamate, amide, carboxylate, sulfonamide, guanyl, guanidinyl, hydrazine, hydrazide, thiohydrazide, and amine;
m is 0, 1, 2 or 3, representing the number of the R¹ substituent(s); and
R² is selected from hydrogen, alkyl, cycloalkyl, and aryl.

According to some embodiments of any of the embodiments described herein, each of Ra and Rb is independently a second reactive/clickable group as described herein.

According to some of these embodiments, Ra and Rb are the same second clickable group.

According to some of these embodiments, each of Ra and Rb is a different second clickable group.

According to some embodiments of any of the embodiments described herein, at least one, or each, of Ra and Rb is or comprises an alkene.

According to some embodiments of any of the embodiments described herein, at least one, or each, of Ra and Rb is or comprises a strained cycloalkyl or a strained unsaturated cycloalkyl.

According to some embodiments of any of the embodiments described herein, at least one of Ra and Rb is or comprises a norbornene.

According to some embodiments of any of the embodiments described herein, each of Ra and Rb independently is or comprises a norbornene.

According to some embodiments of any of the embodiments described herein, R² is alkyl, preferably a lower alkyl such as methyl.

According to some embodiments of any of the embodiments described herein, m is 1, 2, or 3, and at least one of the R¹ substituents is an electron-withdrawing substituent, such as described and defined herein. In exemplary embodiments, R¹ is nitro. In exemplary embodiments, m is 1 and R¹ is nitro.

According to some embodiments of any of the embodiments described herein, the electron-withdrawing substituent is at the meta position with respect to the —L₂-Rb.

According to some embodiments of any of the embodiments described herein, the mechanophoric compound is:

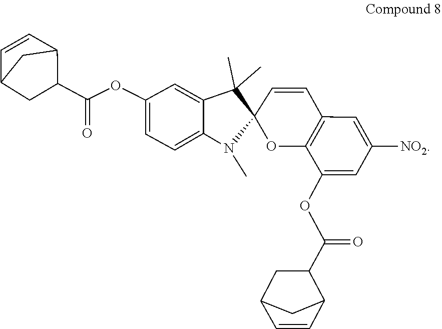 [SMILES: CN1c2ccc(OC(=O)C3CC4C=CC3C4)cc2C(C)(C)[C@]12C=Cc1cc([N+](=O)[O-])cc(OC(=O)C3CC4C=CC3C4)c1O2]

According to some embodiments of any of the embodiments described herein, the obtained mechanophoric matrix or composition-of-matter is capable of exhibiting a force-induced colorimetric change as described herein in any of the respective embodiments.

According an aspect of some of any of the embodiments described herein, there is provided a mechanophoric matrix or composition-of-matter obtainable by the method as described herein in any of the respective embodiments and any combination thereof.

According an aspect of some of any of the embodiments described herein, there is provided a mechanophoric matrix or composition-of-matter (a mechanophore-functionalized matrix or composition-of-matter), comprising a substrate (e.g., a polymeric material, a metallic substrate), having at least one, or a plurality of, a mechanophoric moiety derived from the mechanophoric compound as described herein in any of the respective embodiments and any combination thereof, covalently bound or couples to the substrate via a Click bond. According to some of these embodiments, the Click bond is a moiety formed by a Click reaction, as described herein, between the second clickable group of the mechanophoric compound and a complementary first clickable group present or generated in the substrate.

According to some embodiments of any of the embodiments described herein, the Click bond is selected from a triazole linking moiety, a dihydrotriazole linking moiety, a pyridazine linking moiety, a dihydropyridazine linking moiety, a cycloalkene (e.g., cyclohexene) linking moiety, a cyclodiene linking moiety (e.g., cyclohexadiene), a thioether linking moiety, a vinyl sulfide linking moiety, an alkenyl linking moiety (an alkenylene), an alkyl linking moiety (an alkylene), an ether linking moiety, an alkylene substituted by thiol and/or hydroxy, an aminoether linking moiety, an amide linking moiety, and an imine linking moiety, all being a linking group as described and defined herein.

According to some embodiments of any of the embodiments described herein, the Click bond is a linking moiety obtained by any of the Click reactions as described herein.

According to some embodiments of any of the embodiments described herein, the Click bond is a linking moiety obtained by a 1,4-Michael addition thiol-ene Click reaction as described herein in any of the respective embodiments.

According to some embodiments of any of the embodiments described herein, one of the first and second clickable groups is selected from an azide and a tetrazine, the other one of the first and second clickable groups is selected from alkene or cycloalkene and alkyne, and the Click bond is a triazole linking group (azide with alkyne), a dihydrotriazole linking group (azide with alkene or cycloalkene), a pyridazine linking group (tetrazine with alkyne), or a dihydropyridazine linking group (tetrazine with alkene or cycloalkene); and/or

one of the first and second clickable groups is or comprises a thiol and the other one of the first and second clickable groups is an alkene, a cycloalkene or an alkyne, and the Click bond is a thioether linking moiety (thiol with alkene or cycloalkene) or a vinyl sulfide linking moiety (thiol with alkyne); and/or

one of the first and second clickable groups is a diene and the other one of the first and second clickable groups is a dienophile such as an alkene, a cycloalkene or an alkyne, and the Click bond is or comprises a cycloalkene linking group (e.g., for a diene with an alkene or cycloalkene) or a cyclodiene linking group (e.g., for a diene with an alkyne); and/or

one of the first and second clickable groups is an isonitrile and the other one of the first and second clickable groups is a tetrazine, and the Click bond is a dihydropyridazine linking group; and/or

one of the first and second clickable groups is selected from a strained cycloalkyl, and a strained heteroalicyclic, and the other one of the first and second clickable groups is selected from an alkene and an alkyne, and the Click bond is or comprises a linear or branched alkenyl or alkyl linking group (e.g., a strained cycloalkyl with alkene), a diene or an alkenyl linking group (e.g., cycloalkyl with alkyne), an ether linking group (e.g., epoxide with alkyne), or an aminoether linking group (e.g., aziridine with alkyne); and/or

one of the first and second clickable groups is a strained unsaturated cycloalkyl, and the other one of the first and second clickable groups is selected from an azide and a tetrazine, and the Click bond is a dihydropyridazine linking group (strained cycloalkene with tetrazine) or a triazole linking group (strained cycloalkene with azide); and/or

one of the first and second clickable groups is selected from a carbonyl (including aldehyde), a carboxylate and an amide, and the other one of the first and second clickable groups is an amine, and the Click bond is an imine linking group (carbonyl or aldehyde with amine) or an amide linking group (carboxylate with amine; or amide with amine); and/or

one of the first and second clickable groups is selected from a strained (e.g., unsaturated) cycloalkyl and a strained heteroalicyclic, and the other one of the first and second clickable groups is a nucleophile such as thiol or amine, and the Click bond is a thioether linking group (cycloalkene with thiol) or an amine-containing thioether linking group or thiol-containing thioether (e.g., aziridine with thiol); a hydroxy-containing thioether group (epoxide with thiol); and/or

one of the first and second clickable groups is selected from a strained (e.g., unsaturated) cycloalkyl, and a strained heteroalicyclic, and the other one of the first and second clickable groups is selected from an azide and a tetrazine, and the Click bond is a dihydropyridazine linking group (cycloalkane and tetrazine).

According to some of any of the embodiments described herein, at least one of the mechanophoric moiety is covalently bound via the Click bond to at least two (first) clickable groups present or generated in substrate (e.g., on a least a portion of a surface of the substrate).

According to some embodiments of any of the embodiments described herein, the substrate is or comprises a polymeric material or matrix and the at least one mechanophoric moiety is covalently bound to at least two pendant moieties of the polymeric material. The pendant moieties can be of the same polymeric chain and/or of different polymeric chains of the polymeric material, and the mechanophoric moiety forms crosslinking in the polymeric material. According to some embodiments, the thus crosslinked polymeric material is a gel.

According to some embodiments of any of the embodiments described herein, the mechanophoric matrix or composition-of-matter is capable of exhibiting a force-induced colorimetric change following deformation (e.g., strain application) of at least 1%, or at least 5%, or at least 10%.

According to some embodiments of any of the embodiments described herein, the mechanophoric matrix or composition-of-matter is capable of exhibiting a force-induced colorimetric change following deformation (e.g., strain application) of less than 200%, or less than 100%, or less than 50%, or less than 40%, or lower.

According to some embodiments of any of the embodiments described herein, the force-induced colorimetric change is such that upon application of force, as described herein, the mechanophoric matrix or composition-of-matter features an average B/G color ratio higher by at least 10%, or 30%, or 50%, or 75%, or 100%, or 150%, or 200%, or 300%, or even more (e.g., 1000%), than an average B/G color ratio of an intact mechanophoric matrix, as described herein in any of the respective embodiments.

According to some embodiments, the average B/G color ratio is determined by converting an image of the mechanophoric matrix and an image of the intact mechanophoric matrix to RGB signals and calculating an average B/G color ratio therefrom.

According to some of any of the embodiments described herein, the mechanophoric matrix or composition-of-matter is capable of exhibiting a force-induced colorimetric change following force application of at least 300 Pa, or at least 400 Pa, or at least 450 Pa, or at least 500 Pa, or at least 550 Pa.

According an aspect of some of any of the embodiments described herein, there is provided an article-of-manufacturing comprising the mechanophoric matrix or composition-of-matter as described herein in any of the respective embodiments and any combination thereof. According to some embodiments, upon application of force (as described herein in any of the respective embodiments), the article-of-manufacturing features an average B/G color ratio higher by at least 10%, or 30%, or 50%, or 75%, or 100%, or 150%, or 200%, or 300%, or even more (e.g., 1000%), than an average B/G color ratio of an intact article-of-manufacturing (onto which no force is applied), as described herein in any of the respective embodiments for the mechanophoric matrix or composition-of-matter.

According an aspect of some of any of the embodiments described herein, there is provided a method of determining a presence and/or a level of a force-induced damage in a mechanophoric matrix (or composition-of-matter) or in an article-of-manufacturing comprising the mechanophoric matrix (or composition-of-matter), as described herein in any of the respective embodiments and any combination thereof, the method comprising determining a colorimetric change (e.g., a change in an RGB signal and/or a change in fluorescence and/or a change in light absorption) in the matrix (or composition-of-matter) or the article-of-manufacturing, the colorimetric change being indicative of the presence and/or the level of the force-induced damage in the matrix or article-of-manufacturing.

According to some embodiments of any of the embodiments described herein, the force-induced damage is effected by traction, cutting, compression and/or folding.

According to some embodiments of any of the embodiments described herein, the force-induced damage results from a force application to the mechanophoric matrix is at least 300 Pa, or at least 400 Pa, or at least 450 Pa, or at least 500 Pa, or at least 550 Pa, and/or is lower than 1 MPa or lower than 10 KPa or lower than 5 KPa or lower than 1 KPa.

According to some embodiments of any of the embodiments described herein, the colorimetric change is observed following deformation (e.g., strain application) of at least 1%, or at least 5%, or at least 10%.

According to some embodiments of any of the embodiments described herein, the colorimetric change is observed following deformation (e.g., strain application) lower than 200%, or lower than 100%, or lower than 50%, or lower than 40%, or lower, as described herein.

According an aspect of some of any of the embodiments described herein, there is provided a mechanophoric compound represented by Formula I*,

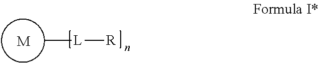

wherein:
Mch is a force-responsive moiety as described herein in any of the respective embodiments and any combination thereof;
L is a linking group or absent, as described herein in any of the respective embodiments and any combination thereof (e.g., for Formula I) or is absent;
R is a clickable group, as described herein in any of the respective embodiments of a second reactive/clickable group, and any combination thereof (e.g., for Formula I); and
n is a positive integer as described herein in any of the respective embodiments and any combination thereof (e.g., for Formula I).

According to some embodiments of any of the embodiments described herein, R in Formula I* is a clickable group or a reactive group capable of participating in a Click reaction by forming a covalent bond with a complementary clickable group, as described herein in any of the respective embodiments and any combination thereof for a first and second reactive or clickable groups.

According to some embodiments of any of the embodiments described herein, Mch in Formula I* is or comprises a spiropyran moiety, as described herein in any of the respective embodiments.

According to some embodiments of any of the embodiments described herein, L in Formula I* is or comprises a carboxy linking group, as described herein in any of the respective embodiments.

According to some embodiments of any of the embodiments described herein, Mch in Formula I* is or comprises a spiropyran, and n is 2.

According to some of these embodiments, the mechanophoric compound is represented by Formula II*:

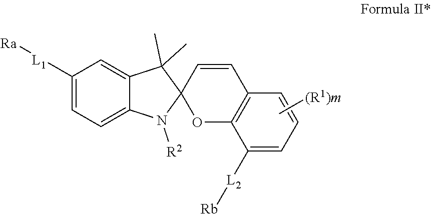

wherein:
Ra and Rb are each independently selected from a substituent or the clickable group, or is absent, wherein at least one of Ra and Rb is the (second, reactive) clickable group, as described herein in any of the respective embodiments (e.g., for Formula II), and wherein the substituent, if present, is selected from alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, carbamate, thiocarbamate, amide, carboxylate, sulfonamide, guanyl, guanidinyl, hydrazine, hydrazide, thiohydrazide, and amine;
L₁ and L₂ are each independently the linking group or is absent, as described herein in any of the respective embodiments and any combination thereof (e.g., for Formula II);
R¹ is a substituent selected from alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, carbamate, thiocarbamate, amide, carboxylate, sulfonamide, guanyl, guanidinyl, hydrazine, hydrazide, thiohydrazide, and amine, and is as described herein in any of the respective embodiments and any combination thereof (e.g., for Formula II);
m is 0, 1, 2 or 3, representing the number of the R¹ substituent(s), as described herein in any of the respective embodiments and any combination thereof (e.g., for Formula II); and
R² is selected from hydrogen, alkyl, cycloalkyl, and aryl, as described herein in any of the respective embodiments and any combination thereof (e.g., for Formula II).

According to some embodiments of any of the embodiments described herein, the compound is Compound 8, as described herein.

According to some embodiments of any of the embodiments described herein, the compound is Compound 7, as described herein.

According to some embodiments of any of the embodiments described herein, the mechanophoric compound is usable in preparing a mechanophore-functionalized matrix or composition-of-matter or an article-of-manufacturing comprising the mechanophore-functionalized matrix or composition-of-matter such as described herein in any of the respective embodiments and any combination thereof or in preparing.

According to an aspect of some embodiments of the present invention there is provided a mechanophoric matrix comprising a metallic material or substrate having at least one mechanophoric moiety covalently attached thereto.

According to some embodiments of any of the embodiments described herein, the mechanophoric moiety is derived from a mechanophoric compound that features a second clickable group, as described herein in any of the respective embodiments and any combination thereof and is covalently bound to the metallic material or substrate via a Click bond formed by a Click reaction between the second clickable group and a complementary first clickable group generated in and/or on the metallic substrate, as described herein.

According to some embodiments of any of the embodiments described herein, the Click reaction, the Click bond, and the first and second clickable groups are as described herein in any of the respective embodiments and any combination thereof.

According to some embodiments of any of the embodiments described herein, the metallic substrate comprises a metallic film featuring the first clickable group.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a scheme presenting exemplary synthetic procedures for the preparation of mono-norbornene and bis-norbornene spiropyran (Compound 7 and Compound 8, respectively), exemplary mechanophoric compounds according to some of the present embodiments.

FIG. 1B presents the exo-configurational isomer and endo-configurational isomer of norbornene acid, a starting material in the preparation of Compound 5 (shown in FIG. 1A).

FIGS. 2A-B are graphs showing a THF-GPC signal from differential refractive-index detector of an exemplary copolymer precursors according to some of the present embodiments, styrene-vinyl benzyl chloride copolymer (PS-Cl) (FIG. 2A) and styrene-vinyl benzyl azide copolymer (PS-N₃) (FIG. 2B).

FIGS. 3A-B are representative images of the breaking point of mono-norbornene-spiropyran-functionalized polystyrene (PS-Mono; FIG. 3A) and bis-norbornene-spiropyran-functionalized polystyrene (PS-Bis; FIG. 3B), exemplary mechanophores-functionalized polymers according to some of the present embodiments, following tensile stress measurements. Scale bar is 0.5 cm.

FIGS. 3C-D present plots showing stress (FIG. 3C) and B/G color ratio (FIG. 3D) as a function of strain in mono-norbornene spiropyran-functionalized polystyrene (blue plots) and bis-norbornene spiropyran-functionalized polystyrene (red plots). A “Blank” polystyrene (PS-N₃) is also presented for comparison (black plots).

FIGS. 3E-F present plots showing the stress (red plot) and B/G ratio (black plot) as a function of uniaxial strain in the exemplary bis-norbornene spiropyran-functionalized polystyrene (FIG. 3E) and in mono-norbornene spiropyran-functionalized polystyrene (FIG. 3F).

FIG. 3G presents a stress-strain plot (blue) of the exemplary bis-norbornene spiropyran-functionalized polystyrene sample, and a corresponding linear fit (dotted red). 5% onset strain is on the edge of the linear regime (vertical grey line).

FIG. 3H presents a comparative scatter plot showing data of the critical activation stress as a function of strain in a spiropyran-based exemplary mechanophoric compounds according to some of the present embodiments, in which the spiropyran-derived mechanophoric moieties are attached to pendant groups of the polymeric chains in comparison with chain-centered spiropyrans incorporated into bulk polymeric materials using previously established methods, as indicated in the Examples section.

FIG. 31 presents a table showing photographs from the time-dependent formation of exemplary bis-norbornene spiropyran-functionalized polystyrene hydrogels, prepared from PS-N₃ (91 mg) and Compound 8 (15 mg) as crosslinker in DCM (0.33 mL) inside a vertically-aligned 5 mL vial incubated at different temperatures, as indicated. The photographs were taken by placing the vial horizontally after its incubation in the vertical position.

FIGS. 4A-C are photographs of the exemplary bis-norbornene spiropyran-functionalized polystyrene according to some of the present embodiments before press (FIG. 4A), after press (FIG. 4B), and after 5 minutes of exposure to white light (FIG. 4C).

FIG. 5A presents comparative plots showing an absorption spectrum of a solution of the exemplary mono-norbornene spiropyran-functionalized polystyrene in MeCN (0.01 mg/mL), demonstrating the change in absorbance over time (0-60 seconds) following exposure to a 16 W ultraviolet light (254 nm).

FIG. 5B is a scheme of an exemplary synthetic procedure for the preparation of a reference spiropyran-functionalized polystyrene (“SP-PS”), which incorporates the spiropyran moiety during the synthesis (polymerization) process as a co-monomer within the polymeric backbone chain (also referred to as “chain-centered”).

FIG. 5C present plots showing the stress (red plot) and B/G ratio (black plot) as a function of uniaxial strain in the reference mechanophoric compound chain-centered SP-PS shown in FIG. 5B, as tested via DMA. The inset images are snapshots from a video analysis taken before (left) and after (right) tensile 60% strain. Stress-strain plot in dog-bone samples and colorimetric changes in the sample (B/G ratio) was measured along the intermediate level, normalized to 2 mm above the clamping area.

FIG. 5D presents representative images showing fluorescence of a dried chain-centered SP-PS thin film shown in FIG. 5B prepared by evaporating droplets of polymer dissolved in DCM on glass slides and then cutting the droplets with a razor blade, before (left) and after incision (right). Images were taken by measuring the fluorescence emission of the film at 575 nm. Scale bar indicates 200 μm.

FIG. 5E is a schematic illustration summarizing the advantageous coupling of an exemplary mechanophoric compound according to some of the present embodiments, bis-norbornene spiropyran (Compound 8) to various substrates and the mechanochromic performance of the obtained exemplary mechanophoric matrices.

FIG. 6A presents an absorption spectra of an aqueous solution of an exemplary tetrazine-modified alginate according to some embodiments of the present invention (3 mg/mL).

FIG. 6B presents an absorption spectra of aqueous solutions containing (4-(1,2,4,5-tetrazin-3-yl)phenyl)methanamine hydrochloride (Tz; “Tetrazine”) at different concentrations. The linear correlation between absorption at 520 nm and the concentration, which allows calculating the molar attenuation coefficient (s) of Tz at 520 nm is shown in the inset.

FIGS. 7A-B are photographs of aqueous solutions of mono-norbornene spiropyran-functionalized tetrazine alginate and bis-norbornene spiropyran-functionalized tetrazine alginate (20 mg/ml) at 4° C. in an inverted vial (FIG. 7A) and in a tilted vial (FIG. 7B).

FIG. 8A presents fluorescence microscopy images of a tetrazine-modified alginate film, a mono-norbornene spiropyran-functionalized tetrazine alginate film, and the exemplary bis-norbornene spiropyran-functionalized tetrazine alginate film, before and after folding the films 180°.

FIG. 8B presents comparative plots showing line profiles quantifying the fluorescence intensity across the folding direction of the bis-norbornene spiropyran-functionalized tetrazine alginate film.

FIG. 8C presents comparative plots showing the fluorescence intensity of bis-norbornene spiropyran-functionalized tetrazine alginate (“ALG-Bis”) thin film measured after bending at increasingly larger angles.

FIG. 8D presents a representative image of the finite element analysis (FEA) model to calculate the stress distribution in an alginate thin film at 135°.

FIG. 8E presents a comparative scatter plot showing correlation between the bending stress and strain from FEA simulations with the experimental bending angle data across the line profile of fluorescence emissions at 575 nm of ALG-Bis.

FIG. 8F presents a schematic illustration of an experimental setup measuring the folding of a film such as a norbornene spiropyran-functionalized tetrazine alginate film according to some embodiments of the present invention, where the film is attached to two glass slides, is folded on itself by moving the glass slides closer one to the other; and the folding angle is measured by a protractor.

FIG. 8G presents a plot showing stress-strain traction of the exemplary bis-norbornene spiropyran-tetrazine alginate hydrogel with a linear fit, generated using the fit function of Matlab™ (dashed red plot).

FIG. 8H presents plots showing the values of the effective strain (von Mises stress) (red squares) and the calculation time over the number of elements for a folding angle of 1000 (black squares).

FIG. 8I presents plots showing the volumetric distribution of the effective (von Mises) stresses (red squares) and standard energy density (SED) over the folding angle of the film (black squares).

FIG. 8J presents representative image of the finite element analysis (FEA) model showing different steps of the film folding process (top) and slice view on the Y-Z plane stresses (bottom), showing effective stress in the film.

FIGS. 9A-C are fluorescence microscopy images of bis-norbornene spiropyran alginate film before compression (FIG. 9A), after compression (FIG. 9B), and after exposure to white light for 1 hour (FIG. 9C) under a red fluorescent protein (RFP) filter. Scale bar is 200 μm.

FIGS. 10A-B are photographs of bis-norbornene spiropyran-functionalized alginate-acrylamide composite film before (FIG. 10A) and after (FIG. 10B) 180° bending. Scale bar is 300 μm.

FIG. 11 presents a plot showing fluorescence intensity at 575 nm of bis-norbornene spiropyran-alginate-acrylamide composite film under various bending angles. The obtained data were plotted, with the eye guideline represented by a red line.

FIGS. 12A-B presents IR spectra of varying amounts of bis-norbornene spiropyran pelleted with 200 mg KBr and their correlation coefficient at 2960 cm⁻¹ wavenumber (FIG. 12A), and of raw wool and wool functionalized by the exemplary bis-norbornene spiropyran (FIG. 12B).

FIG. 13A presents an absorption spectra of varying concentrations of (3-mercaptopropyl) methyldimethoxysilane (MPTES) following its reaction with Ellman solution. The linear correlation between its absorption at 410 nm to the concentration, which allows calculating the molar attenuation coefficient (s), is shown in the inset.

FIG. 13B is a bar graph showing the content of thiol groups in and/or on the surface of the wool [mM per gram of wool] in different stages of the functionalization process.

FIG. 14A is a photograph of a bis-norbornene spiropyran-conjugated TEOS-wool string following MPTES treatment, that was subsequently washed with DCM to remove unreacted bis-norbornene spiropyran after dipping in the solution. The wool fibers were then twisted to apply shear force in the same direction as the yarn's twist. The arrow points to the activated bis-norbornene spiropyran-conjugated TEOS-wool.

FIGS. 14B-C are photographs (FIG. 14B) and fluorescence microscopy images (FIG. 14C) of the bis-norbornene spiropyran-conjugated TEOS-wool before and after compression inside a one cm diameter cylindrical container.

FIG. 14D is a comparative plot presenting the measured stress, strain, and fluorescence of the bis-norbornene spiropyran-conjugated TEOS-wool samples prepared as described in FIGS. 14B-C. The strain was calculated from the change in the sample thickness as a result of compression at different measured pressures. At each pressure, the sample was removed to measure fluorescence at 575 nm.

FIG. 14E presents a fluorescence (575 nm) and brightfield image of bis-norbornene spiropyran-conjugated TEOS-wool following activation by scissor cutting. The scale bar indicates 200 μm.

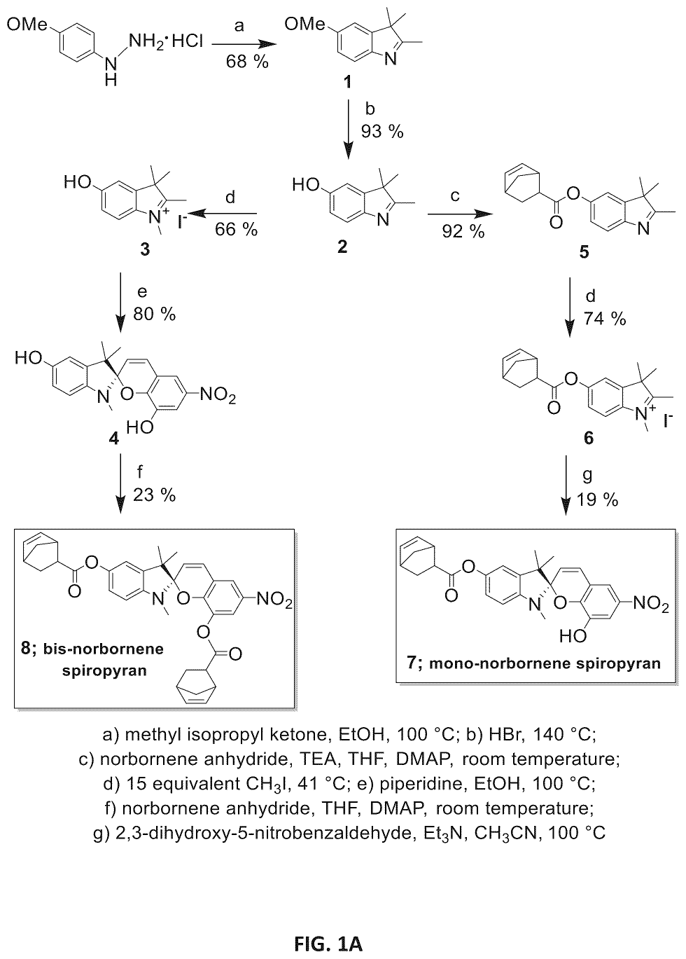

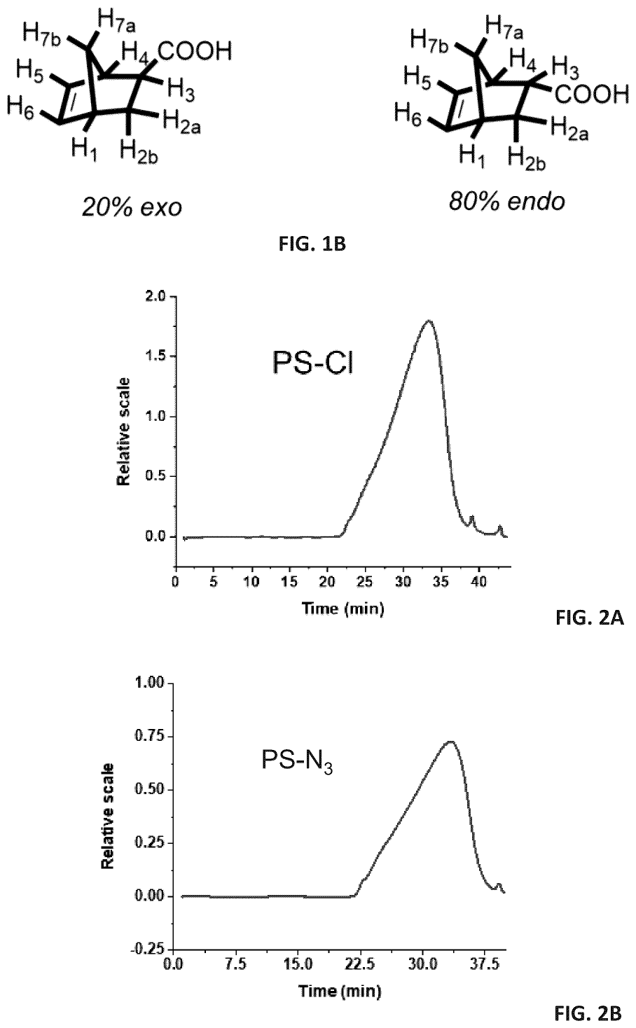

FIG. 14F is a schematic illustration of an exemplary preparation method (“dip-conjugating”) of an exemplary mechanophore-conjugated natural substrate, bis-norbornene spiropyran-functionalized alpaca wool, from washing and substituting the alpaca wool with MPTES (steps 1 and 2) followed by its functionalization with an exemplary reactive group is bis-norbornene spiropyran (Compound 8 of FIG. 1A) under UV light (step 3).

FIG. 15A presents a schematic illustration of a two-step procedure for functionalizing a gold-coating steel foil with thiols and respective images of the gold-coated steel and the thiol-functionalized final product.

FIG. 15B presents goniometer images of a drop of water and its respective contact angle as measured in a wettability test on an exemplary thiol-substituted gold surface before (up, left), during (up, center) and after (up, right) functionalization with an exemplary mechanophoric compound, bis-norbornene spiropyran (Compound 8), and the mechanophoric compound-functionalized metallic substance following UV-activation (bottom).

FIGS. 16A-B present a schematic illustration comparing the method for preparing mechanophoric matrices according to some of any of the respective embodiments described herein using Click chemistry (FIG. 16B), with Background art methods (FIG. 16A) for the preparation of a (e.g., polymer-based) mechanophoric matrices.

### DESCRIPTION OF SPECIFIC EMBODIMENTS OF THE INVENTION

The present invention, in some embodiments thereof, relates to material science, and, more particularly, but not exclusively, to a novel methodology for converting varying matrices into mechanophoric matrices, to novel mechanophoric compounds and novel matrices obtained thereby and to applications thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

As discussed in further details in the Background section, current methodologies for integrating mechanophores into bulk materials, particularly polymers, face significant challenges that limit their practical use. These include difficulties in precisely incorporating mechanophores into the matrix, which lead to poor sensitivity to mechanical strain, and stability issues during processing. Additionally, current methods often require high concentrations of mechanophores and complex procedures, which restrict the types of polymers that can be used. As a result, while mechanophores have offered a range of promising applications, their integration into materials had remained a major challenge, and strategies like conjugational crosslinking, which could address these challenges, had not yet been explored.

The present inventors have devised an approach for circumventing the challenges associated with (e.g., in-situ) conjugation of mechanophores to mechanophoric matrices/substrates. The present inventors have conceived applying a quantitative and biorthogonal conjugation of a mechanophoric compound to a matrix or substrate while employing Click chemistry as a means to universally convert various matrices (e.g., synthetic and natural polymeric materials, metallic substrates) into mechanophoric, force-sensitive materials with colorimetric indicators.

To test this strategic approach for mechanophore incorporation into bulk materials and networks (matrices) or substrates, Click chemistry reactions (herein also being referred to as Click addition or generally as Click chemistry) such as Michael addition reactions, were used, and novel norbornene-substituted spiropyran derivatives were used as exemplary mechanophoric compounds. By utilizing reactive groups that are intrinsically present or are generated in and/or the substrate, and which specifically react with a norbornene group through various Click reactions, a variety of substrates such as bulk polymers and metallic substrates can be functionalized with mechanophores, post-polymerization and/or post-processing.

A novel methodology and novel compounds that allows to graft mechanophores (e.g., spiropyrans) onto various mechanophoric matrices/substrates and/or articles comprising same is described herein. For polymeric materials, the described methodology is by a post-functionalization step instead of the commonly practiced method (e.g., blending a mechanophore with grown polymers into polymer melts). See, for example, FIGS. 16A-B. By using this approach, the mechanophore is grafted onto both natural and synthetic materials through several different bio-orthogonal Click chemistries (e.g., Michael addition to the thiol group in proteins or thiolated gold films; Click reaction to polymers or carbohydrates (e.g., polysaccharides) with azide or tetrazine moieties). This allows functionalizing the substrate (e.g., polymeric material, metallic substrate) by simply contacting it with a solution of the respective mechanophore.

As demonstrated in the Examples section that follows, the present inventors have designed and successfully prepared and practiced a novel strategy for converting raw materials (substrates; matrices) into mechanophore-functionalized (also being referred to as “mechanophore-conjugated”) substrates, by, e.g., Click reaction (see, e.g., FIG. 16B), which allows performing the reaction under mild conditions, without restricting its reversible mechano-activation properties. These provide a simple approach for the incorporation of mechanophores into various substrates, such as polystyrene, alginate hydrogels, wool, silk, 3D-printed nylon, epoxy resins and metal foils, thus providing a wide range of mechanochromic materials (see, e.g., FIG. 5E) that allows turning conventional articles into force-detecting sensors with minimal effort and cost. Furthermore, surprisingly, these mechanophoric matrices exhibit high sensitivity, with a colorimetric change that starts at deformation (e.g., strain) which is as low as 10%, or even 5% or even lower.

The applications of the methods and compounds disclosed herein extend beyond conventional products, as colorimetric force sensors significantly enhanced the performance, maintenance, and safety of the tested natural and synthetic components. This strategy opens up the possibilities of new mechanochromic materials with various scientific and commercial implications in various fields (e.g., biology, mechanical engineering).

Embodiments of the present invention therefore relate to newly designed mechanophoric compounds, to methods of preparing mechanophoric matrices using these compounds and to mechanophoric articles-of-manufacturing obtained thereby.

Herein, the term “mechanophore” or “mechanophoric compound” encompasses any force-responsive or force-sensitive, mono- or polyvalent molecule. Mechanophores are molecular units that undergo selective bond scission in response to an external force to provide a measureable signal to correlate to the applied force for a targeted chemical response. Mechanisms involved in this bond scission include, but are not limited to, targeted homolytic cleavage of the weaker bonds in a structure, dative bond scission for specialized bonds with inorganic elements, cycloreversion to break a cyclic ring and revert back to two separate molecules, and electrocyclic ring opening in which then ring opening retains the single molecule structure.

As known in the art, color-changing mechanophores (also being referred to in the art as mechanochromophore or a mechanochromic material) exhibit a change in their optical properties, as can be directly detected and evaluated by, e.g., optical measurements, even if the degree of activation is not high.

Herein, the term “mechanophoric compound” encompasses any mechanophore molecular unit which comprises at least one moiety, preferably at least two moieties, that can covalently bind to a substrate and/or article according to some of any of the embodiments of the present invention. In some of any of the embodiments of the present invention, the moiety comprises at least one spiropyran or derivative thereof. Upon covalent coupling or conjugation of a mechanophoric compound to the substrate, a mechanophoric moiety is generated, representing the mechanophoric portion of the mechanophoric compound that remains upon the covalent coupling.

According to some of any of the embodiments described herein, the mechanophoric compound as described herein is a mechanochromic compound.

The term “Click reaction” generally refers to a Click chemistry reaction that is highly energetically favored, stereospecific, insensitive towards oxygen and water, carried out in high chemical yield (e.g., near quantitative conversion and mild reaction conditions), and is typically performed under mild reaction conditions (e.g., temperature lower than 100° C.; in water, under air, at room temperature, and in the absence of catalysts), and occurs between two groups that are generally unreactive except with respect to each other.

The term “quantitative” describes a chemical reaction that is performed in at least 80%, preferably at least 90%, or at least 95%, or at least 98%, or at 100%, yield with respect to at least one of the reactants.

Herein, Click reactions encompass any addition and/or addition-elimination reactions that meet the above-mentioned criteria of a “Click reaction”.

Non-limiting examples of Click reactions according to some of the present embodiments include Huisgen 1,3-dipolar cycloaddition, a 1,4-Michael addition thiol-ene reaction, a Diels-Alder reaction, an inverse electron demand Diels-Alder reaction, an isonitrile-tetrazine cycloaddition, a nucleophilic substitution (e.g., between small strained cycloalkyl or heteroalicyclic and alkyne), an amide bond formation (e.g., between carbonyl, carboxylate, C-amide and amine), strain-promoted alkyne-nitrogen cycloaddition (SPANC), nucleophilic ring opening of strained heteroalicyclic (e.g., aziridine, epoxide), and strain-promoted azide-alkyne cycloaddition (SPAAC).

As used herein and in the art, the phrase “Huisgen 1,3-dipolar cycloaddition” describes a chemical reaction that typically forms five-membered rings through the interaction of a 1,3-dipole (such as an azide) with a dipolarophile.

Herein and in the art, “dipolarophile” describes a chemical compound that can react with a 1,3-dipole in a cycloaddition reaction to form a ring structure. Non-limiting examples include alkenes, including cycloalkenes, and alkynes.

As used herein and in the art, the phrase “thiol-ene reaction” or a 1,4-Michael addition thiol-ene reaction describes a chemical reaction between a thiol and an alkene, as these are defined herein, typically resulting in the formation of a thioether moiety via a radical-mediated addition process.

As used herein and in the art, the phrase “Diels-Alder reaction” describes a chemical reaction between a conjugated diene and a dienophile, forming a cycloalkyl such as a cycloalkene (e.g., cyclohexene) ring, which widely used in organic synthesis for constructing six-membered rings.

As used herein and in the art, the term “dienophile” describes a chemical compound containing a single double bond or triple bond that reacts with a diene in cycloaddition reactions, forming a cyclic product. Non-limiting examples include alkenes, cycloalkenes, (e.g., norbornene), alkynes, imides, nitriles, and quinones.

As used herein and in the art, the term “diene” describes a chemical compound containing two double bonds which can participate in cycloaddition reactions such as Diels-Alder reactions. Non-limiting examples include butadiene, furane, and cyclopentadiene.

As used herein and in the art, the phrase “inverse electron demand Diels-Alder reaction” describes a variant of the Diels-Alder reaction where the electron-poor diene (e.g., tetrazine) reacts with an electron-rich dienophile (e.g., norbornene, cyclooctyne, trans-cyclooctene), facilitating the formation of a cycloalkene such as cyclohexene.

As used herein and in the art, the phrase “isonitrile-tetrazine cycloaddition” describes a chemical reaction where an isonitrile group reacts with a tetrazine to form a stable (e.g., unsaturated) heteroalicyclic adduct.

As used herein and in the art, the phrase “nucleophilic substitution” describes a chemical reaction in which a nucleophile such as thiol or amine displaces a leaving group on an electrophile, such as a small strained cycloalkyl or heteroalicyclic.

As used herein and in the art, the phrase “amide bond formation” describes a chemical reaction that creates an amide linkage by coupling, e.g., a carboxylate group (e.g., or amide such as C-amide, as defined herein, with an amine.

As used herein and in the art, the phrase “strain-promoted alkyne-nitrogen cycloaddition” (SPANC) describes a chemical reaction between a strained alkene or alkyne (e.g., norbornene, cyclooctyne) and a nitrogen-containing group (e.g., azide) to form a stable heteroalicyclic.

As used herein, the phrase “nucleophilic ring opening of strained heteroalicyclic” describes a reaction where a nucleophile (e.g., thiol) attacks and opens a strained heteroalicyclic, such as an aziridine or epoxide, resulting in the formation of a more stable heteroalicyclic product.

As used herein, the phrase “strain-promoted azide-alkyne cycloaddition” (SPAAC) describes a chemical reaction between an azide and a strained alkene (e.g., norbornene) or a strained alkyne (e.g., bicyclo[6.1.0]non-4-yne (BCN), dibenzocyclooctyne (DBCO)).

Herein throughout, the term “alkene” encompasses linear alkenes and cyclic alkenes (cycloalkenes) unless otherwise indicated.

According to some of any of the embodiments described herein, a Click reaction as defined herein is effected upon contacting one clickable group with a complementary clickable group in accordance with the Click reactions as described herein. Herein, a pair of a clickable group and a complementary clickable group that can react therewith in a Click reaction are also being referred to as “clickable pairs”, or as “complementary clickable groups” or as a “first and second reactive groups” or as a “first and second clickable groups” or as “or as a “first and second complementary clickable groups”.

Herein, a reactive group that is capable of participating in a Click reaction is also referred to as “a clickable group” or as a “clickable reactive group” or as a first or second reactive groups, and encompasses any of the groups described herein that participate in any of the Click reactions as described herein.

Pairs of first and second reactive or clickable groups, which are described herein as complementary with one another, encompass any combination of the reactive groups that participate in any of the Click reactions as described herein.

According to some of any of the embodiments described herein, whenever a (first or second) clickable or reactive group is referred to, such a group can be any of the groups described herein as participating in any of the Click reactions as described herein.

Whenever a complementary (first or second) clickable or reactive group is referred to, such a group can be the respective other group that participate in the respective Click reaction as described herein.

Herein, a first and second reactive or clickable groups are complementary to one another such that whenever a first reactive group is described as complementary to a second reactive group, the second reactive group is also complementary to the first reactive group.

According to some of any of the embodiments described herein, exemplary clickable group that can be used as a first and a second reactive (clickable) group as described herein include, but are not limited to, an azide, a tetrazine, an alkene, including cycloalkene, a strained cycloalkane, a strained cycloalkene (e.g., norbornene, cyclopropene, cyclobutene), an alkyne, a thiol, a carbonyl, a carboxylate, an amide (e.g., a C-amide), an amine, an isonitrile, an aziridine, a heteroalicyclic (e.g., a strained heteroalicyclic such as epoxide or aziridine), a strained cycloalkyl (e.g., a cyclopropane, a cyclobutane), and fused (e.g., strained) heteroalicyclic and/or cycloalkyl rings.

Pairs of a first and a second reactive (clickable) groups, which are complementary to one another as described herein are in accordance with any of the Click reactions as described herein and/or with any of the respective embodiments as described herein.

Such pairs of complementary first and second reactive (clickable) groups form a Click bond, which is a moiety formed as a result of the Click reactions.

Herein throughout, the phrase “Click bond” describes the moiety formed upon the click reaction by which the mechanophore is linked to the substrate.

According to some embodiments of any of the embodiments described herein, the Click bond is a linking moiety or group obtained by a Click reaction (e.g., 1,4-Michael addition thiol-ene reaction) as described herein in any of the respective embodiments.

Depending on the selected first and second reactive (clickable) complementary groups, the Click bond can be or comprise, for example, a triazole linking moiety, a dihydrotriazole linking moiety, a pyridazine linking moiety, a dihydropyridazine linking moiety, a cycloalkene (e.g., cyclohexene)linking moiety, a cyclodiene linking moiety, a thioether linking moiety, a vinyl sulfide linking moiety, an alkenyl linking moiety, an alkylene linking moiety, an ether linking moiety, an aminoether linking moiety, an amide linking moiety, and an imine linking moiety, all being a linking group as described and defined herein.

According to some embodiments of any of the embodiments described herein, one of the first and second reactive (e.g., clickable) groups is selected from an azide and a tetrazine, and the other one of the first and second reactive (e.g., clickable) groups is selected from an alkene (e.g., cycloalkene) and an alkyne. According to some of these embodiments, the Click bond formed between the first and the second reactive (e.g., clickable) groups is or comprises a triazole linking moiety (azide with alkyne), a dihydrotriazole linking moiety (azide with alkene), a pyridazine linking moiety (tetrazine with alkyne), or a dihydropyridazine linking moiety (tetrazine with alkene).

According to some embodiments of any of the embodiments described herein, one of the first and second reactive (e.g., clickable) groups is or comprises a thiol and the other one of the first and second reactive (e.g., clickable) groups is selected from an alkene and an alkyne. According to some of these embodiments, the Click bond formed between the first and the second reactive (e.g., clickable) groups is or comprises a thioether linking moiety (thiol with alkene) or a vinyl sulfide linking moiety (thiol with alkyne).

Herein throughout, the terms “linking moiety” and “linking group” are used interchangeably, unless otherwise indicated.

According to some embodiments of any of the embodiments described herein, one of the first and second reactive (e.g., clickable) groups is or comprises a diene and the other one of the first and second reactive (e.g., clickable) groups is or comprises a dienophile, as described and defined herein. According to some of these embodiments, the Click bond formed between the first and the second reactive (e.g., clickable) groups is or comprises a cycloalkene linking moiety (e.g., for diene with alkene) or cyclodiene linking moiety (e.g., for diene with alkyne). In preferred embodiments, the cycloalkene or cyclodiene is a 5-membered or a 6-membered ring. As Click reactions provide thermodynamically stable compounds, cycloalkene or cyclodiene are typically 5-membered ring or 6-membered ring.

According to some embodiments of any of the embodiments described herein, one of the first and second reactive (e.g., clickable) groups is an isonitrile and the other one of the first and second reactive (e.g., clickable) groups is a tetrazine. According to some of these embodiments, the Click bond formed between the first and the second reactive (e.g., clickable) groups is or comprises a dihydropyridazine linking moiety.

According to some embodiments of any of the embodiments described herein, one of the first and second reactive (e.g., clickable) groups is selected from a strained (e.g., unsaturated) cycloalkyl (e.g., norbornene, cyclopropyl, cyclobutyl), and a strained heteroalicyclic (e.g., aziridine, epoxide), and the other one of said first and second reactive (e.g., clickable) groups is selected from alkene (preferably a strained unsaturated cycloalkyl) and alkyne. According to some of these embodiments, the Click bond formed between the first and the second reactive (e.g., clickable) groups is or comprises a linear or branched alkyl linking moiety (e.g., cyclopropyl or cyclobutyl with alkene), a linear or branched diene or alkenyl linking moiety (e.g., a cyclopropyl or a cyclobutyl with alkyne), an ether linking moiety (e.g., via ring-opening of epoxide with alkyne), or an aminoether linking moiety (e.g., via ring-opening of aziridine with alkyne).

According to some embodiments of any of the embodiments described herein, one of the first and second reactive (e.g., clickable) groups is selected from a strained cycloalkyl and a strained heteroalicyclic, and the other one of the first and second reactive (e.g., clickable) groups is selected from an azide and a tetrazine. According to some of these embodiments, the Click bond formed between the first and the second reactive (e.g., clickable) groups is or comprises a dihydropyridazine linking moiety (norbornene with tetrazine), or a triazole linking moiety (norbornene with azide).

According to some embodiments of any of the embodiments described herein, one of the first and second reactive (e.g., clickable) groups is selected from a carbonyl (including an aldehyde), a carboxylate and an amide, and the other one of the first and second reactive (e.g., clickable) groups is or comprises an amine. According to some of these embodiments, the Click bond formed between the first and the second reactive (e.g., clickable) groups is or comprises an imine linking moiety (carbonyl or aldehyde with amine) or an amide linking moiety (e.g., carboxylate with amine; or amide with amine).

According to some embodiments of any of the embodiments described herein, one of the first and second reactive (e.g., clickable) groups is selected from a strained (e.g., unsaturated) cycloalkyl and a strained heteroalicyclic (e.g., an aziridine, an epoxide), and the other one of said first and second reactive (e.g., clickable) groups is or comprises a nucleophile (e.g., a thiol). According to some of these embodiments, the Click bond formed between the first and the second reactive (e.g., clickable) groups is or comprises a thioether linking moiety (e.g., via cycloalkyl with thiol; via ring-opening of aziridine with thiol; or via ring-opening of epoxide with thiol).

According to some embodiments of any of the embodiments described herein, one of said first and second reactive (e.g., clickable) groups is selected from a strained cycloalkyl, and a strained heteroalicyclic, and the other one of the first and second reactive (e.g., clickable) groups is selected from an azide and a tetrazine. According to some of these embodiments, the Click bond formed between the first and the second reactive (e.g., clickable) groups is or comprises a dihydropyridazine linking moiety.

According to some of any of the embodiments described herein, one of the first and second reactive (clickable) groups is selected from an azide and a tetrazine, and the other one of the first and second reactive (clickable) groups (a complementary reactive or clickable group) is or comprises a strained unsaturated cycloalkyl.

According to some of any of the embodiments described herein, one of the first and second reactive (e.g., clickable) groups is or comprises a thiol, and the other one of the first and second reactive (e.g., clickable) groups (a complementary reactive or clickable group) is or comprises a strained unsaturated cycloalkyl.

According to some of any of the embodiments described herein, one of the first and second reactive (e.g., clickable) groups is selected from a strained cycloalkyl, and a strained heteroalicyclic, and the other one of the first and second reactive (e.g., clickable) groups (a complementary reactive or clickable group) is or comprises a strained unsaturated cycloalkyl.

Herein throughout, the terms “cycloalkene” and “unsaturated cycloalkyl” are used interchangeably to describe a cycloalkyl as described and defined herein, which includes at least one, preferably one, unsaturated (double) bond.

As used herein and in the art, the term “strained” describes a molecule or moiety that features increased internal energy due to non-ideal bond angles and/or torsional strain, often resulting from small ring sizes and/or steric hindrance.

As used herein and in the art, the phrase “strained cycloalkyl” describes a cycloalkyl or unsaturated cycloalkyl (cycloalkene) as defined herein, which is strained as defined herein.

Non-limiting examples of strained cycloalkyls include cyclopropyl, cyclobutyl, cubane, and fused cycloalkyl rings.

Non-limiting examples of strained unsaturated cycloalkyls include cyclopropene, cyclobutene, norbornene (bicyclo[2.2.1]heptane), and fused rings comprising unsaturated cycloalkyls.

Non-limiting examples of strained heteroalicyclic include aziridine, epoxide (oxirane), and fused rings comprising heteroalicyclic compounds.

### Mechanophoric Compounds:

According to an aspect of some embodiments of the present invention there is provided a mechanophoric compound which is or comprises a force-responsive or force-sensitive moiety featuring one or more reactive group(s) capable of participating in a Click reaction (i.e., one or more clickable groups) as defined herein in any of the respective embodiments.

As used herein, the phrase “reactive group” generally describes a chemical group or moiety which can readily react with another moiety to thereby form one or more chemical bond(s), preferably one or more covalent bond(s). Herein, the reactive group is a clickable group that is capable of participating with a complementary clickable group in any of the Click reactions as described herein.

The mechanophoric compounds according to some of the present embodiments can be collectively represented by Formula I (also referred to herein as Formula I*):

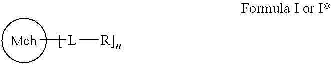

wherein:
Mch is a force-responsive moiety (e.g., a moiety or skeleton derived from a force-responsive or force-sensitive compound or molecule);
L is a linking group or absent;
R is the clickable group; and
n is a positive integer, or an integer greater than 1.

By “moiety derived from a force-sensitive or force-responsive compound” it is meant a moiety that features all the structural features of the force-sensitive or force-responsive (mechanophoric) compound from which it is derived, yet, one or more (represented by the variable “n”) positions of the compound are substituted by the R reactive/clickable group. The R group(s) can substitute a position of the mechanophoric compound which is otherwise unsubstituted in the mechanophoric compound from which the moiety is derived, or can replace a substituent present in the of the mechanophoric compound from which the moiety is derived, as long as this replacement does not affect the force-sensitivity or force-responsiveness. In other words, the moiety has a major portion of the compound from which it is derived, except from the additional/replacement R substituent(s).

For example, a moiety that comprises a spiropyran is derived from a force-sensitive or force-responsive spiropyran, and includes the spiropyran skeleton that accounts for the force-sensitivity or responsivity.

The mechanophoric compound described herein is derived from a force-responsive or force-sensitive compound or molecule, that is, it features a skeleton of a force-responsive compound, represented by the force-responsive moiety denoted as Mch in Formula I and is substituted by one or more clickable groups, denoted as R in Formula I. In some embodiments, the position of the R clickable group(s) substituents are selected so as to maintain the mechanophoric performance upon being coupled to a substrate as described herein.

Representative examples of force-responsive compounds from which Mch can be derived include, but are not limited to, spiropyrans (SP), dithienylethenes, spirolactams, dianthracenes, merocyanines (MC), naphthopyrans (NP), bis[3H]-naphtho[2,1-b]pyran thiophenes, bis-naphthopyrans, copper complexes, disulfides, diarylbibenzothiophenonyls (DABBT), diarylbibenzofuranones (DABBF), tetraarylsuccinonitriles (TASN), ladderenes, oxazimes, fulminic acids, each being optionally substituted by one or more substituents other than the [L—R].

Herein, the term “spiropyrans (SP)” describes a class of mechanophores composed of a spiropyran ring system that is closed in its non-photoactive state. Upon exposure to light (UV radiation) or mechanical stress, the ring opens, resulting in a color change from colorless to typically yellow or red, forming the colored merocyanine (MC) structure.

Herein, the term “merocyanines (MC)” describes a class of mechanophores that are the colored, open-ring form of spiropyrans. These molecules exhibit a color change, typically from yellow to colorless, when they revert to the spiropyran form upon the removal of light or mechanical stress.

Herein, the term “dithienylethenes” describes a class of mechanophores consisting of two thiophene rings connected by a central ethene unit. The system undergoes a reversible color change from colorless (closed-ring form) to colored (typically blue or red) upon exposure to light (UV radiation) or mechanical stress, due to isomerization between the open and closed forms.

Herein, the term “spirolactams” describes a class of mechanophores that feature a spirolactam ring, a cyclic amide linked to a spiro junction. This structure undergoes a color change from colorless to red or pink when subjected to mechanical stress or chemical stimuli, such as a pH change, which causes ring opening.

Herein, the term “dianthracenes” describes a class of mechanophores composed of two anthracene units connected by a central bond. Exposure to light (UV radiation) or mechanical stress can trigger a color change, typically from yellow to colorless, due to the reversible breaking of the central bond.

Herein, the term “naphthopyrans (NP)” describes a class of mechanophores that undergo a reversible color change when exposed to UV light or mechanical stress, typically transitioning from colorless to yellow or orange, due to the opening of the pyran ring.

Herein, bis[3H]-naphtho[2,1-b]pyran thiophenes describes a class of mechanophores that consist of a naphthopyran structure fused with thiophene rings. These compounds undergo a color change from colorless to colored (often yellow or red) upon exposure to UV light or mechanical stress, due to ring-opening reactions.

Herein, the term “bis-naphthopyrans” describes a class of mechanophores composed of two naphthopyran units. These molecules exhibit a reversible color change from colorless to colored (typically yellow or orange) upon exposure to light (UV radiation) or mechanical stress, resulting from the opening of the pyran rings.

Herein, the term “copper complexes” describes a class of mechanophores that involve copper ions coordinated with organic ligands. These complexes may undergo a color change due to changes in coordination geometry or oxidation state when subjected to mechanical stress or chemical stimuli.

Herein, the term “disulfides” describes a class of mechanophores featuring an S-S bond. Mechanical stress or redox reactions can trigger the cleavage of the disulfide bond, leading to a structural change that may result in a color change or alteration in material properties.

Herein, the term “diarylbibenzothiophenonyls (DABBT)” describes a class of mechanophores consisting of a diaryl-substituted bibenzothiophenonyl structure. These compounds can undergo a color change when exposed to mechanical stress, typically due to the breaking or reformation of bonds within the thiophene system.

Herein, the term “diarylbibenzofuranones (DABBF)” describes a class of mechanophores that consist of diaryl-substituted bibenzofuranone structures. These molecules exhibit color changes when mechanical stress induces bond rearrangement or cleavage within the furanone system.

Herein, the term “tetraarylsuccinonitriles (TASN)” describes a class of mechanophores that have a central succinonitrile core with four aryl substituents. These compounds undergo color changes upon mechanical stress, typically resulting from the breaking of the central carbon-carbon bonds.

Herein, the term “ladderenes” describes a class of mechanophores that consist of fused bicyclic or polycyclic structures, resembling a ladder-like framework. These compounds are characterized by their unique mechanical properties, where the alternating single and double bonds in the ladder structure can undergo controlled scission under mechanical stress.

Herein, the term “oxazimes” describes a class of mechanophores that contain an oxime group (—C═NOH) linked to an aromatic or aliphatic structure. Upon exposure to mechanical stress or light, these molecules can undergo a color change due to isomerization or cleavage of the oxime bond.

Herein, the term “fulminic acids” describes a class of mechanophores characterized by the —C≡NO functional group. These compounds are highly reactive and can undergo decomposition or isomerization under mechanical stress, potentially resulting in a color change or the release of gases.

Representative examples of R clickable groups include, but are not limited to, an azide, a tetrazine, an alkene, a cycloalkene, an alkyne, a thiol, a carbonyl, a carboxylate, a C-amide, an amine, an isonitrile, an aziridine, a heteroalicyclic, a cycloalkyl, an epoxide, a norbornene, a cyclopropane, a cyclopropane, a cyclobutene, a cyclobutane, and fused heteroalicyclic and/or cycloalkyl rings, or any of the clickable groups as described herein (e.g., a second reactive or clickable group).

According to some of any of the embodiments described herein, the clickable group R is attached to the force-responsive moiety Mch either directly (when L is absent) or via a linking group L.

Representative examples of linking group L include, but are not limited to, an alkylene, an alkenylene, an alkynylene, a cycloalkyl linking group, an aryl linking group, a heteroaryl linking group, a heteroalicyclic linking group, —O—, —S—, an alkoxylene linking group, an aryloxylene linking group, a thioalkoxylene linking group, a thioaryloxylene linking group, a sulfinyl linking group, a sulfonyl linking group, a sulfonate linking group, a sulfate linking group, a cyano linking group, a phosphonyl linking group, a phosphinyl linking group, a carbonyl linking group, a thiocarbonyl linking group, an urea linking group, a thiourea linking group, a carbamyl linking group, a thiocarbamyl linking group, an amido linking group, a carboxy linking group, a sulfonamido linking group, a guanyl linking group, a guanidinyl linking group, a hydrazine linking group, a hydrazide linking group, a thiohydrazide linking group, and an amine linking group, as these groups are described and defined herein.

According to some of any of the embodiments described herein, L is or comprises a carboxy linking group (e.g., L is —O—C(═O)— or —O—C(═O)—).

According to some of any of the embodiments described herein, L is a linking group of no more than 4, or no more than 3, or no more than 2 atoms in length, such that the clickable group is in close spatial proximity to the Mch skeleton of the mechanophoric compound. This may allow the Mch portion to be in close spatial proximity to the substrate to which it is coupled to form a mechanophoric matrix or composition-of-matter, as described herein.

According to some of any of the embodiments described herein, the mechanophoric compound is derived from a spiropyran such that Mch is or comprises a spiropyran, that is, it features a spiropyran skeleton.

Mechanophoric compounds derived from spiropyrans can be collectively represented by Formula II (which is also referred to herein as Formula II*):

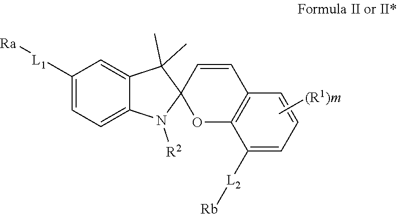

wherein:

Ra and Rb are each independently the clickable group, as described herein in any of the respective embodiments and any combination thereof, or a substituent as described herein, or is absent, wherein at least one of Ra and Rb is a clickable group (a second reactive group or a second clickable group as described herein); L₁ and L₂ are each independently a linking group, as described herein in any of the respective embodiments and any combination thereof, or is absent, such that Ra and/or Rb are attached directly to the respective phenyl ring;
R¹ is a substituent of the benzopyran portion of the spiropyran;
m is 0, 1, 2, or 3, representing the number of the R¹ substituents; and
R² (a substituent of the indoline portion of the spiropyran) is selected from hydrogen, alkyl, alkenyl, alkynyl, cycloalkyl, aryl, heteroaryl, and heteroalicyclic or from hydrogen, alkyl, cycloalkyl and aryl, or from hydrogen, alkyl and cycloalkyl, or from hydrogen and alkyl, preferably a lower alkyl as described herein.

According to some of any of the embodiments described herein, one of Ra and Rb is the clickable group as described herein (e.g., a second clickable or reactive group). In some such embodiments, one of Ra and Rb is the clickable group and the other one is absent.

In some embodiments, Ra is absent and Li is absent or Ra is a substituent of the force-responsive moiety Mch and Li is absent. In some embodiments, Rb is absent and L₂ is absent or Rb is a substituent of the force-responsive moiety Mch and L₂ is absent.

Non-limiting examples for substituents of the force-responsive moiety Mch include alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, carbamate, thiocarbamate, amide, carboxylate, sulfonamide, guanyl, guanidinyl, hydrazine, hydrazide, thiohydrazide, and amine, as these are defined herein.

According to some of any of the embodiments described herein, the clickable group is selected capable of participating in at least one type of Click reaction as defined herein.

According to some of any of the embodiments described herein, the clickable group is a second clickable group or a second reactive group as described herein in any of the respective embodiments and any combination thereof.

According to some embodiments of any of the embodiments described herein, the second reactive/clickable group is not an acrylic group (e.g., an acrylate, a methacrylate, an acrylamide or a methacrylamide group) or does not comprise an acrylic group.

According to some embodiments of any of the embodiments described herein, the mechanophoric compound is devoid of an acrylic group.

According to some of any of the embodiments described herein, Ra and Rb can each independently be any one of the clickable groups (e.g., a second reactive or clickable group) as described herein.

In some of any of the embodiments described herein, Ra and Rb are each independently any one of the clickable groups (e.g., a second reactive or clickable group) as described herein.

In some of any of the embodiments described herein, Ra and Rb can be the same of different.

In some of any of the embodiments described herein, the Ra and/or Rb clickable groups is/are selected to suit respective (complementary) one or more (first) clickable groups of a substrate to be modified.

According to some of any of the embodiments described herein, the clickable group R is or comprises an alkene.

In some of any of the embodiments described herein, at least one, or each, of Ra and Rb is an alkene.

In some of any of the embodiments described herein, Ra and Rb each independently is or comprises an alkene.

According to some of any of the embodiments described herein, the alkene is a cycloalkene (also being referred to herein interchangeably as “unsaturated cycloalkyl”).

According to some of any of the embodiments described herein, the clickable group R is or comprises an unsaturated cycloalkyl as defined herein.

In some of any of the embodiments described herein, Ra and Rb each independently is or comprises an unsaturated cycloalkyl.

In some of any of the embodiments described herein, Ra is or comprises an alkene and Rb is or comprises an unsaturated cycloalkyl, or vice versa.

In some of any of the embodiments described herein, Ra is or comprises an unsaturated cycloalkyl and Rb is absent, or vice versa.

In some of any of the embodiments described herein, Ra is or comprises an unsaturated cycloalkyl and Rb is or comprises a different clickable group, or vice versa. According to some of any of the embodiments described herein, the alkene is a strained cycloalkene.

According to some of any of the embodiments described herein, the clickable group R is or comprises a strained unsaturated cycloalkyl as defined herein.

In some of any of the embodiments described herein, Ra and Rb each independently is or comprises a strained unsaturated cycloalkyl.

In some of any of the embodiments described herein, Ra is or comprises an alkene and Rb is or comprises a strained unsaturated cycloalkyl, or vice versa.

In some of any of the embodiments described herein, Ra is or comprises an unsaturated cycloalkyl and Rb is or comprises a strained unsaturated cycloalkyl, or vice versa.

In some of any of the embodiments described herein, Ra is or comprises a strained unsaturated cycloalkyl and Rb is absent, or vice versa.

In some of any of the embodiments described herein, Ra is or comprises a strained unsaturated cycloalkyl and Rb is or comprises a different clickable group, or vice versa.

According to some of any of the embodiments described herein, the second reactive group is or comprises a strained cycloalkyl or a strained unsaturated cycloalkyl. In some of any of the embodiments described herein, the second reactive group is or comprises a norbornene.

According to some of any of the embodiments described herein, the clickable group is or comprises a norbornene.

In some of any of the embodiments described herein, at least one of Ra and Rb is or comprises a norbornene.

In some of any of the embodiments described herein, each of Ra and Rb is or comprises a norbornene.

In some of any of the embodiments described herein, Ra and Rb each independently is a norbornene.

In some of any of the embodiments described herein, Ra is or comprises an alkene and Rb is or comprises a norbornene, or vice versa.

In some of any of the embodiments described herein, Ra is or comprises a strained unsaturated cycloalkyl and Rb is or comprises a norbornene, or vice versa.

In some of any of the embodiments described herein, Ra is or comprises a norbornene and Rb is or comprises a different clickable group, or vice versa.

In some of any of the embodiments described herein, Ra is or comprises a norbornene and Rb is absent, or vice versa.

In some of any of the embodiments described herein, R² is hydrogen.

According to some of any of the embodiments described herein, R² is an alkyl, a cycloalkyl or an aryl.

In some of any of the embodiments described herein, R² is an alkyl or a cycloalkyl.

In some of any of the embodiments described herein, R² is an alkyl.

In some of any of the embodiments described herein, R² is a lower alkyl of 1 to 6, or of 1 to 4, or of one, carbon atoms in length.

In preferred embodiments, R² is a linear alkyl. In some of any of the embodiments described herein, R² is a linear alkyl of 1 to 6, or of 1 to 4, or of one, carbon atoms in length.

In preferred embodiments, R² is an unsubstituted alkyl. In preferred embodiments, R² is an unsubstituted linear alkyl. In more preferred embodiments, R² is an unsubstituted linear alkyl of 1 to 6, or of 1 to 4 carbon atoms in length, for example, methyl, ethyl, propyl, or butyl.

In exemplary embodiments, R² is methyl.

According to some of any of the embodiments described herein, m is 0, R¹ is absent, and the benzopyran portion of the spiropyran is unsubstituted.

According to some of any of the embodiments described herein, m is other than 0, and can be 1, 2, or 3, such that the benzopyran portion of the spiropyran is substituted by one or more substituents.

According to some embodiments, one of R¹ substituents is an electron-withdrawing group, and is also referred to herein as an “electron-withdrawing substituent”.

According to some of any of the embodiments described herein, the electron-withdrawing substituent is at the meta position with respect to the —L₂-Rb.

The phrase “electron-withdrawing group” (also referred to herein interchangeably as “electron-withdrawing moiety” or “electron-withdrawing group”), as used herein, generally describes a chemical group in a molecule, which can draw electrons away from another part of the molecule. The distance over which the electron-withdrawing group can exert its effect, namely the number of bonds over which the electron-withdrawing effect spans, can be extended by conjugated π-electron systems. Non-limiting examples of suitable electron-withdrawing groups include, but are not limited to, nitro, ammonium ions (positively charged amino cations, such as pyridinium ions), carboxy-containing groups (e.g., aldehydes, ketones, esters), sulfonate, sulfate, nitrile, cyano, and trihaoalkyls (e.g., trifluoromethyl (—CF₃)), as these terms are defined herein.

In some of any of the embodiments described herein, Ra is a clickable group and L₁ is a carboxy-containing group as described herein (e.g., —O—C═O—).

In some of any of the embodiments described herein, Rb is a clickable group and L₂ is a carboxy-containing group as described herein.

In some of any of the embodiments described herein, Ra is a clickable group and L₁ is a carboxy-containing group as described herein and Rb is a clickable group and L₂ is a carboxy-containing group as described herein.

In some of any of the embodiments described herein, R¹ is an electron withdrawing group and is at the meta position, and m is 1; In some of any of the embodiments described herein, R¹ is nitro and is at the meta position, and m is 1.

In some of any of the embodiments described herein, R¹ is an electron withdrawing group and is at the meta position, m is 1, Rb is a clickable group and L₂ is a carboxy-containing group as described herein.

In some of any of the embodiments described herein, R¹ is nitro and is at the meta position, m is 1, Rb is a clickable group and L₂ is a carboxy-containing group as described herein.

In some of any of the embodiments described herein, R² is an alkyl such as methyl, Ra is a clickable group and Li is a carboxy-containing group as described herein.

In some of any of the embodiments described herein, R² is methyl, m is 1, Ra is a clickable group and Li is a carboxy-containing group as described herein.

In some of any of the embodiments described herein, R¹ is an electron withdrawing group such as nitro and is at the meta position, and m is 1, and Ra and Rb are each independently a clickable group as described herein in any of the respective embodiments. In some of these embodiments, each of Ra and Rb is a strained unsaturated cycloalkyl (e.g., a norbornene).

In some of any of the embodiments described herein, R¹ is an electron withdrawing group such as nitro and is at the meta position, and m is 1, and L₁ and L₂ are each independently a carboxy-containing group as described herein.

In some of any of the embodiments described herein, R¹ is an electron withdrawing group such as nitro and is at the meta position, and m is 1, Rb is a clickable group and Li is a carboxy-containing group as described herein, Li is absent and Ra is a substituent such as hydroxyl.

In some of any of the embodiments described herein, R¹ is an electron withdrawing group such as nitro and is at the meta position, and m is 1, Rb is a clickable group and Li is a carboxy-containing group as described herein.

In some of any of the embodiments described herein, R² is an alkyl such as methyl, Ra is a clickable group and Li is a carboxy-containing group as described herein.

In some of any of the embodiments described herein, R² is an alkyl such as methyl, Ra is a clickable group and Li is a carboxy-containing group as described herein, m is 1, L₂ is absent and Rb is a substituent such as hydroxyl.

In some of any of the embodiments described herein, R¹ is an electron withdrawing group such as nitro and is at the meta position, and m is 1, Rb is a clickable group and L₂ is a carboxy-containing group as described herein.

In some of any of the embodiments described herein, R¹ is an electron withdrawing group such as nitro and is at the meta position, m is 1, Ra is a clickable group and Li is a carboxy-containing group as described herein, and Rb is a clickable group and L₂ is a carboxy-containing group as described herein. In some of these embodiments, each of Ra and Rb is a strained unsaturated cycloalkyl such as norbornene.

In some of any of these embodiments, R² is an alkyl, for example, methyl.

According to some of any of the embodiments described herein, Ra is a clickable group, Li is a linking moiety which is or comprises a carboxy linking group, R² is an alkyl, for example, methyl, m is 1, R¹ is an electron withdrawing group and is at the meta position with respect to the benzopyran portion of the spiropyran, L₂ is absent and Rb is a hydroxyl group.

According to some of any of the embodiments described herein, Formula I and Formula I* are the same. Alternatively, Formula I and Formula I* differ from one another by the type of the force-responsive moiety, the type of the clickable group, the number n of clickable groups, the presence and/or type of the linking group, and/or by the presence and/or type of any other variable.

According to some of any of the embodiments described herein, Formula II and Formula II* are the same. Alternatively, Formula II and Formula II* differ from one another by the type of the force-responsive moiety, the type of the clickable group, the number n of clickable groups, the presence and/or type of the linking group, and/or by the presence and/or type of any other variable.

According to some of any of the embodiments described herein, Formulae I and II describe mechanophoric compounds when used for preparing mechanophoric matrices or compositions-of-matter as described herein and Formulae I* and II* describe mechanophoric compounds per se.

According to some of any of these embodiments, the clickable group is a strained cycloalkene as defined herein, for example norbornene.

An exemplary mechanophoric compound according to these embodiments is Compound 7:

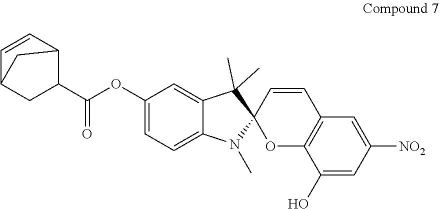 [SMILES: CN1c2ccc(OC(=O)C3CC4C=CC3C4)cc2C(C)(C)[C@]12C=Cc1cc([N+](=O)[O-])cc(O)c1O2]

According to some of any of the embodiments described herein, Rb is a clickable group, L₂ is a linking moiety which is or comprises a carboxy linking group, R² is an alkyl, for example, methyl, m is 1, R¹ is an electron withdrawing group and is at the meta position with respect to the benzopyran portion of the spiropyran, L₁ is absent and Ra is a hydroxyl group.

An exemplary mechanophoric compound according to these embodiments is Compound 7a:

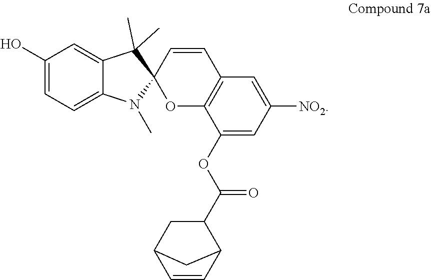 [SMILES: CN1c2ccc(O)cc2C(C)(C)[C@]12C=Cc1cc([N+](=O)[O-])cc(OC(=O)C3CC4C=CC3C4)c1O2]

According to some of these embodiments, the clickable group is a strained cycloalkene as defined herein, for example, norbornene.

According to some of any of the embodiments described herein, Ra and Rb are each a clickable group, L₁ and L₂ are each a linking moiety which is or comprises a carboxy linking group, R² is an alkyl, for example, methyl, m is 1, and R¹ is an electron withdrawing group and is at the meta position with respect to the benzopyran portion of the spiropyran.

An exemplary mechanophoric compound according to these embodiments is Compound 8:

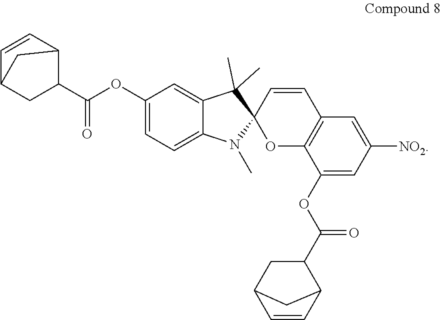 [SMILES: CN1c2ccc(OC(=O)C3CC4C=CC3C4)cc2C(C)(C)[C@]12C=Cc1cc([N+](=O)[O-])cc(OC(=O)C3CC4C=CC3C4)c1O2]

### Method:

According to some embodiments of the present invention, a mechanophoric compound as described herein in any of the respective embodiments is usable in preparing variable mechanophoric matrices, by enabling simple coupling, via Click chemistry, between the one or more of the clickable groups to corresponding, complementary reactive groups in a substrate of interest. Thus, the mechanophoric compounds as described herein are introduced to corresponding substrates via a chemical reaction that can be performed under mild conditions and without excessive catalysts and/or reagents, to thereby maintain both the mechanophoric nature of the introduced mechanophoric moiety and the inherent properties and structure of the substrate.

According to an aspect of some embodiments of the present invention there is provided a method of preparing a mechanophoric matrix (a mechanophoric composition-of-matter). According to some embodiments of this aspect of the present embodiments, the method is effected by contacting a substrate (e.g., a polymeric material, a metallic substrate or as described herein in any of the respective embodiments) that features a plurality of a first reactive group (e.g., a first clickable group) with a mechanophoric compound that features at least one of a second reactive group (e.g., a first clickable group), wherein the first and second reactive groups are capable of undergoing a chemical reaction that leads to a covalent bond formation therebetween (e.g., are complementary clickable groups).

According to some of any of the embodiments described herein, the chemical reaction is a Click reaction, as described and defined herein in any of the respective embodiments. According to some of these embodiments, the first reactive group is a first clickable group as described and defined herein in any of the respective embodiments, and the second reactive group is a second clickable group (which is complementary with the first clickable group) as described and defined herein in any of the respective embodiments, such that the first and second reactive or clickable groups are capable of undergoing a Click reaction with one another to thereby form a covalent bond (a Click bond as described and defined herein in any of the respective embodiments) therebetween, thereby preparing the mechanophoric matrix or composition-of-matter.

According to some of any of the embodiments described herein, the Click reaction features at least one, or all, of the following: it is performed in the absence of a catalyst; it is performed at a temperature lower than 100, or lower than 80, or lower than 60, or lower than 40, ° C.; it is a quantitative reaction; and it is performed at ambient (e.g., oxygen-containing) environment.

Any of the Click reactions, first and second reactive or clickable groups, and Click bonds, as described herein, are contemplated.

According to some of any of the embodiments described herein, the method further comprises, prior to contacting the substrate and the mechanophoric compound, generating at least one, or a plurality of, a first reactive group in and/or on a surface of the substrate, for example, on at least a portion of the surface of the substrate. In some of these embodiments, generating the first reactive group(s) is effected by contacting the substrate with a reagent that comprises the first clickable group, or with a reagent that forms the first clickable group when reacted with intrinsic functional groups in and/or on the substrate.

For example, a reagent that comprises a clickable group and a functional group that can act as a leaving group can be reacted with nucleophilic groups intrinsic to the substrate to thereby form a substrate featuring the first clickable group.

As used herein and as known in the art, the phrase “leaving group” describes an atom or group that becomes detached from an atom in what is considered to be the residual or main part of the substrate in specified reactions (e.g., in nucleophilic substitutions).

Any other reagents are contemplated.

In some of any of the embodiments described herein, the method comprises, prior to generating the first clickable group(s), generating in and/or on the substrate functional groups that can react with the reagent as described herein, for example, nucleophilic groups.

According to some of any of the embodiments described herein, the method is effected by generating one or more, for example, a plurality, of a first reactive (clickable) group in and/or on the substrate (e.g., on at least a portion of a surface of the substrate) by contacting the substrate with a reagent that comprises a clickable group as described herein; and contacting the substrate that comprises the first clickable group(s) with a mechanophoric compound as described herein in any of the respective embodiments, to thereby prepare the mechanophoric matrix.

In alternative embodiments, the clickable groups are intrinsically present in and/or on the substrate.

In some of any of the embodiments described herein, the method comprises, prior to contacting the substrate and the mechanophoric compound or prior to generating the first clickable group(s) in and/or on the substrate, cleaning the substrate (e.g., by immersing the substrate in a solution; to remove foreign objects).

According to some of any of the embodiments described herein, contacting the substrate with a mechanophoric compound as these are described herein in any of the respective embodiments) is performed under conditions that allow the chemical reaction (that leads to a covalent bond formation between the first and second reactive groups; e.g. the Click reaction as described herein in any of the respective embodiments) to occur.

According to some of any of the embodiments described herein, the Click reaction features at least one, or at least two, or all, of the following:
is performed in the absence of a catalyst as described herein;
is performed at a temperature lower than 100, or lower than 80, or lower than 60, or lower than 40, ° C.;
is a quantitative reaction; and is performed at ambient (e.g., oxygen-containing) environment.

According to some of any of the embodiments described herein, the contacting is performed in the absence of a catalyst. In some of any of the embodiments described herein, the contacting is performed in the absence of a chemical catalyst. In some of any of the embodiments described herein, the contacting is performed in the absence of a metal and/or metal ion catalyst. In some of any of the embodiments described herein, the contacting is performed in the absence of a photoinitiator.

According to some of any of the embodiments described herein, the contacting is performed at a temperature lower than 100, or lower than 80, or lower than 60, or lower than 40, ° C., for example, at ambient temperature (e.g., 15-25, or 20-25, ° C.) or a temperature that ranges from −20 to 100, or −20 to 80, or −20 to 60, or −20 to 40, or 0 to 100, or 0 to 80, or 0 to 60, or 0 to 40, or 20 to 100, or 20 to 80, or 20 to 60, or 20 to 40, ° C., including any intermediate values and subranges therebetween.

According to some of any of the embodiments described herein, the contacting is effected/performed in the absence of a cross-linker (other than the mechanophoric compound).

According to some of any of the embodiments described herein, the contacting is effected/performed in the absence of a peroxide.

According to some of any of the embodiments described herein, the substrate comprises a polyolefin, and the contacting is performed in the absence of a crosslinker and/or a peroxide.

According to some of any of the embodiments described herein, the contacting comprises contacting the substrate with a solution comprising the mechanophoric compound. In some of any of the embodiments described herein, the contacting comprises contacting the substrate with an aqueous solution comprising the mechanophoric compound.

According to some of any of the embodiments described herein, the solution comprises water and/or a polar organic solvent, for example, an alcohol (e.g., ethanol), dichloromethane (DCM), acetone, acetonitrile, dimethylformamide (DMF), any other polar organic solvent, or any combination of the foregoing. According to some of any of the embodiments described herein, the organic solvent is a water-miscible organic solvent.

According to some of any of the embodiments described herein, the chemical reaction is a quantitative reaction as defined herein in any of the respective embodiments.

According to some of any of the embodiments described herein, the chemical reaction is performed under ambient environment (e.g., oxygen containing environment, for example, air. In some of any of the embodiments described herein, the chemical reaction is not performed under an inert atmosphere (e.g., inert nitrogen and/or argon atmosphere).

According to some embodiments of any of the embodiments described herein, there is provided a method of preparing a mechanophoric matrix comprising a polymeric material according to some of any of the embodiments described herein, the method comprises contacting the polymeric material with a reagent comprising a clickable group/s, to thereby provide a polymeric material that comprises at least one of a plurality of the clickable group; and contacting the polymeric material that comprises at least one of a plurality of the clickable group with a mechanophoric compound according to some of any of the respective embodiments, to thereby obtain the mechanophoric matrix comprising the polymeric material.

In some of any of the embodiments described herein, the method further comprises co-polymerizing the obtained mechanophoric matrix by contacting it with a plurality of monomers under conditions that induce co-polymerization, to thereby generate a composite material.

In some of any of the embodiments described herein, the method further comprises co-polymerizing a polymeric material that features a plurality of first clickable groups by contacting it with a plurality of monomers under conditions that induce co-polymerization, to thereby generate a composite material. In some of any of the embodiments described herein, the method further comprises co-polymerizing a plurality of monomers that form the polymeric material in which each monomer features one or more of a first clickable groups by contacting it with a plurality of co-monomers under conditions that induce co-polymerization, to thereby generate a composite polymeric material that features a plurality of first clickable groups. In such embodiments, the substrate is a polymeric composite material.

Conditions that induce co-polymerization depend on the nature of the co-polymerized monomers, and may include a presence of a photoinitiator and irradiation, in cases of photo-induced polymerization such as free radical polymerization.

### Mechanophoric Compositions-of-Matter:

According to an aspect of some embodiments of the present invention there is provided a mechanophoric matrix obtainable or obtained by the method as described herein in any of the respective embodiments.

According an aspect of some of any of the embodiments described herein, there is provided a mechanophoric matrix or composition-of-matter (a mechanophore-functionalized matrix or composition-of-matter), which comprises a substrate (e.g., a polymeric material, a metallic substrate), as described and defined herein in any of the respective embodiments, having at least one, or a plurality of, a mechanophoric moiety derived from the mechanophoric compound as described herein in any of the respective embodiments and any combination thereof (e.g., Formula I as described herein), covalently bound to the substrate via a Click bond as described and defined herein in any of the respective embodiments. According to some of these embodiments, the Click bond is formed by a Click reaction between the second clickable group of the mechanophoric compound as described and defined herein in any of the respective embodiments and a complementary first clickable group as described and defined herein in any of the respective embodiments which is present or generated in the substrate, as described herein in any of the respective embodiments.

According to some of any of the embodiments described herein, at least one of the mechanophoric moiety features two second clickable groups is covalently bound via two respective Click bonds to two (first) clickable groups present or generated in substrate (e.g., on a least a portion of a surface of the substrate).

According to some embodiments of any of the embodiments described herein, the substrate is or comprises a polymeric material and at least one mechanophoric moiety is covalently bound to at least two pendant moieties of the polymeric material. The pendant moieties can be of the same polymeric chain and/or of different polymeric chains of the polymeric material, and the mechanophoric moiety forms crosslinking in the polymeric material. According to some embodiments, the thus crosslinked polymeric material is a gel.

According to some of any of the embodiments described herein, the substrate is or comprises a polymeric material or a metallic material or substance.

According to some of any of the embodiments described herein, the substrate is or comprises a polymeric material. In some of any of these embodiments, the mechanophoric moiety is covalently bound or coupled to the polymeric material via a side chain and/or a backbone unit of the polymeric material. In some of any of these embodiments, the mechanophoric moiety is covalently bound or coupled to the polymeric material such that it does not form a part of the polymeric backbone chain, that is, it is not a backbone unit (e.g., a co-monomer) of the polymeric backbone chain.

The term “polymeric material” describes a polymeric matrix or network that is already polymerized, namely, it is non-polymerizable and/or non-crosslinkable and/or non-vulcanizable under the conditions used to generate it.

The term “polymer” or “polymeric material” or “polymeric matrix” as used herein encompasses homopolymers, co-polymers and block-co-polymers, and any substance or matrix comprising linear, branched, dendritic and/or star-shaped network thereof.

According to some of any of the embodiments described herein, the polymeric material comprises a natural polymeric substance (also being referred to as a natural polymer or natural polymeric material) and/or a synthetic polymeric substance (also being referred to as a synthetic polymer or a synthetic polymeric material).

Exemplary synthetic polymeric materials include, without limitations, epoxy resins, polystyrenes, polyolefins, polyesters, polyamides (e.g., nylon), polyamines, polyurethanes, acrylic polymers, polyvinyl polymers, rubbers, silicones, co-polymers or block-co-polymers comprising two or more of the foregoing, and any combination thereof.

According to some of any of the embodiments described herein, the polymeric material comprises a synthetic polymer selected from epoxy resins and polyurethanes.

Epoxy resins and polyurethanes are commonly used in the formulation of paints and coatings. The paints may also comprise pigments, fillers, and other additives to enhance their performance and aesthetic qualities, providing tailored solutions for a wide range of surfaces and environments.

According to some of any of the embodiments described herein, the synthetic polymeric material is or comprises a polystyrene (e.g., a styrene-vinyl benzyl chloride copolymer).

In some of any of the embodiments described herein, the synthetic polymeric material is devoid of residual amounts of a catalyst or any other reagent or solvent used for the polymerization/processing of the polymer.

Exemplary natural polymeric materials include, without limitation, polysaccharides and proteinaceous substances.

As used herein and in the art, the term “polysaccharides” describes a class of complex carbohydrates composed of long chains of monosaccharide units bound together by glycosidic linkages. These macromolecules serve various functions, including energy storage and structural roles in living organisms. Non-limiting examples for polysaccharides include alginate, cellulose, chitin, starch, and glycogen.

According to some of any of the embodiments described herein, the substrate is or comprises a natural polymeric material, and the polymeric material is or comprises an alginate. According to some of these embodiments, the first reactive group is or comprises a tetrazine.

In some of any of the embodiments described herein, the substrate is or comprises a non-fibrous proteinaceous substance.

As used herein and in the art, the phrase “non-fibrous proteinaceous substance” describes a type of protein that is typically globular or compact and soluble, rather than elongated and fibrous. Non-limiting examples for non-fibrous proteinaceous substances include enzymes (e.g., amylase, protease), antibodies (e.g., immunoglobulins), and hormones (e.g., insulin).

As used herein and in the art, the phrase “fibrous proteinaceous substance” describes a type of protein that is typically elongated and insoluble, forming the structural framework of cells and tissues in organisms. These substances are characterized by their fibrous morphology and are integral to the mechanical strength and elasticity of various biological structures. Non-limiting examples for fibrous proteinaceous substances include collagen, elastin, keratin (e.g., wool), and fibroin (e.g., silk).

According to some of any of the embodiments described herein, the natural polymeric substance is a fibrous proteinaceous substance.

According to some of any of the embodiments described herein, the natural polymeric substance is wool. Non-limiting examples of wool include sheep wool (e.g., Merino wool, Shetland wool, lambswool), alpaca wool (e.g., Huacaya, Suri), cashmere wool, mohair wool, Angora wool, llama wool, yak wool, camel hair, Qiviut wool, and Vicuna wool. According exemplary embodiments, the wool is alpaca wool.

According to some of any of the embodiments described herein, the substrate is or comprises a metallic material or substance. A metallic material or substance describes a material or substance that comprises one or metals, optionally only in a portion thereof. The metal can be any metal including earth metals, transition metals, noble metals and else.

In some embodiments of any of the embodiments described herein, the substrate comprises a metal film. In some embodiments of any of the embodiments described herein, the metal is or comprises gold. In some embodiments of any of the embodiments described herein, the substrate comprises a gold film.

According to some of these embodiments, the metallic material or substance comprises a metal (e.g., stainless steel) having a metallic film (e.g., gold film) deposited on at least a portion thereof. According to some of these embodiments, the metallic film is a gold film that features at least one, and preferably a plurality of, the first reactive (clickable) group (e.g., thiol group).

According to some embodiments of any of the embodiments described herein, the metallic substance comprises gold. According to some of these embodiments, the gold features at least one, preferably a plurality of the first reactive (clickable) group (e.g., thiol group).

### Metallic Substrates:

According to an aspect of some embodiments of the present invention there is provided a mechanophoric matrix comprising a metallic substrate having at least one mechanophoric moiety covalently attached thereto.

The metallic substrate can be or comprise a metallic material or substance as described herein.

According to some embodiments of any of the embodiments described herein, the mechanophoric moiety is derived from a mechanophoric compound that features a second clickable group as described herein in any of the respective embodiments and any combination thereof, and is covalently bound or coupled to said metallic substrate via a Click bond as described herein in any of the respective embodiments and any combination thereof formed by a Click reaction as described herein in any of the respective embodiments and any combination thereof between the second clickable group and a complementary first clickable group as described herein in any of the respective embodiments and any combination thereof generated in and/or on the metallic substrate.

According to some embodiments of any of the embodiments described herein, the metallic substrate comprises a metallic film featuring the second clickable group, as described herein in any of the respective embodiments and any combination thereof.

According to some embodiments of any of the embodiments described herein, there is provided a method of preparing a mechanophoric matrix comprising a metallic substrate according to some of any of the embodiments described herein, which is effected by contacting the metallic substrate with a reagent comprising a clickable group/s, to thereby provide a metallic substrate that comprises at least one of a plurality of the clickable group, as described herein in any of the respective embodiments and any combination thereof for a first reactive or clickable group; and contacting the metallic substrate that feature at least one or a plurality of the first clickable group with a mechanophoric compound as described herein in any of the respective embodiments and any combination thereof (e.g., of Formula I, I*, II or II*, as described herein), to thereby obtain the mechanophoric matrix comprising the metallic substance.

In some of any of the embodiments described herein, the metallic substrate comprises gold, and the reagent comprising a clickable group/s is thiol-based reagent (e.g., dithiothreitol), such that the clickable group is thiol. In some of any of the embodiments described herein, the method comprises, prior to contacting the metallic substance that comprises at least one or a plurality of the clickable group with the mechanophoric compound, washing the metallic substance that comprises at least one of a plurality of the clickable group (e.g., with water or an aqueous solution).

### Applications:

According to an aspect of some embodiments of the present invention there is provided an article-of-manufacturing comprising the mechanophoric matrix or composition-of-matter as described herein in any of the respective embodiments and any combination thereof.

According to an aspect of some embodiments of the present invention there is provided a mechanophoric matrix or composition-of-matter as described herein, usable in determining a presence and/or a level of a force-induced damage in the substrate or in an article-of-manufacturing comprising same.

According to some of any of the embodiments described herein, determining a presence and/or a level of a force-induced damage is effected by determining a colorimetric change of a mechanophoric matrix as defined herein upon application of force, that is, determining a force-induced colorimetric change.

According to some of any of the embodiments described herein, the mechanophoric matrix or article-of-manufacturing comprising same as described in any of the respective embodiments, exhibits a force-induced change in an optical property (e.g., a colorimetric change). In some such embodiments, the optical property is a colorimetric change (e.g., a change in a UV-Vis absorption). In some such embodiments, the optical property is a change in fluorescence. Methods for measuring a UV-Vis absorption and fluorescence are well known in the art.

According to some of any of the embodiments described herein, the force-induced change in an optical property (e.g., a change is visible color or in fluorescence). According to some of any of the embodiments described herein, the change in the optical property is reversible upon exposure to a light source. In some of any of the embodiments described herein, the force-induced change in an optical property dissipates upon exposure to a light source (e.g., the optical property is reverted to the optical property before the force-induced change, or the change in the optical property after the exposure to a light source in comparison with the optical property before the force-induced change is lower than the force-induced change in an optical property before exposure to the light source). In some such embodiments, the light source is a UV light (up to 350 nm). In some such embodiments, the light source is a white light source (350-700 nm).

According to some of any of the embodiments described herein, upon application of force, the mechanophoric matrix exhibits a deformation, the deformation leading to the colorimetric change in the mechanophoric matrix. The deformation of the mechanophoric matrix can be expressed in terms of force which is being applied onto the mechanophoric matrix.

Non-limiting examples for actions that result in application of force and/or deformation of the mechanophoric matrix include traction, compression, torsion, cutting, shearing, folding, stretching, bending, crushing, indentation, tearing, and impact.

According to some of any of the embodiments described herein, the application of force is effected by an action selected from traction, compression, torsion, cutting, and folding.

According to some of any of the embodiments described herein, the force-induced damage is effected by cutting, traction, compression and/or folding.

According to some of any of the embodiments described herein, the mechanophoric moiety is a mechanochromophore as defined herein in any of the respective embodiments, such that the mechanophoric matrix exhibits a colorimetric change upon an application of force on the mechanophoric matrix as described herein in any of the respective embodiments.

As used herein, the phrase “colorimetric change” encompasses any change which is visual as perceived by the human visual system (e.g., has a maximal wavelength which is within the visual spectrum) and/or is detectable using an optical device (e.g., a fluorescent device, a spectrophotometer, a colorimeter).

According to some of any of the embodiments described herein, upon an application of force, the mechanophoric matrix features an average RGB signal, when determined as described herein, different from an average RGB signal of an intact mechanophoric matrix. As used herein and in the art, the phrase “RGB signal” describes a representation of color information in terms of the intensities of red (R), green (G), and blue (B) light, as perceived by the human visual system or by an optical device, wherein each value ranges from 0 to 255 and signifies the intensity of the respective color component.

In order to convert a color to a RGB signal, one should perform a process for translating a given color (i.e., a color that forms upon the colorimetric change), into its corresponding RGB values. Non-limiting examples for translating a color to RGB signal include using a colorimeter or a spectrophotometer, and using a camera (e.g., a DSLR camera, a digital camera, a smartphone camera).

When using a digital camera, translating a color to RGB signal further includes extracting RGB values from the photo using an image editing software (e.g., Adobe Photoshop™, Microsoft Paint™) or online tools in combination with a respective color picker tool.

A colorimeter typically provides color measurements in non-RGB color space (e.g., CIELAB or CIE XYZ color space). Converting it to RGB values can be performed using a conversion tool which is typically included in the software provided by the colorimeter manufacturer.

A spectrophotometer provides spectral data, often in the form of reflectance or transmittance values. These can be converted to RGB values, e.g., using the stepwise conversion: (i) to a non-RGB color space ((e.g., CIELAB or CIE XYZ color space), which is typically included in the software provided by the spectrophotometer manufacturer, followed by (ii) its conversion to RGB using software and/or online tools, as described herein.

According to some of any of the embodiments described herein, the RGB signal of the mechanophoric matrix is higher by at least 10%, or by at least 20%, or by 30%, or by 50%, or by 75%, or by 100%, or by 200%, or even higher (e.g., by 1000%), than an average RGB signal of an intact mechanophoric matrix.

According to some of any of the embodiments described herein, the RGB signal is a B/G color ratio. A B/G color ratio is determined based on the optical techniques described herein for a RGB signal.

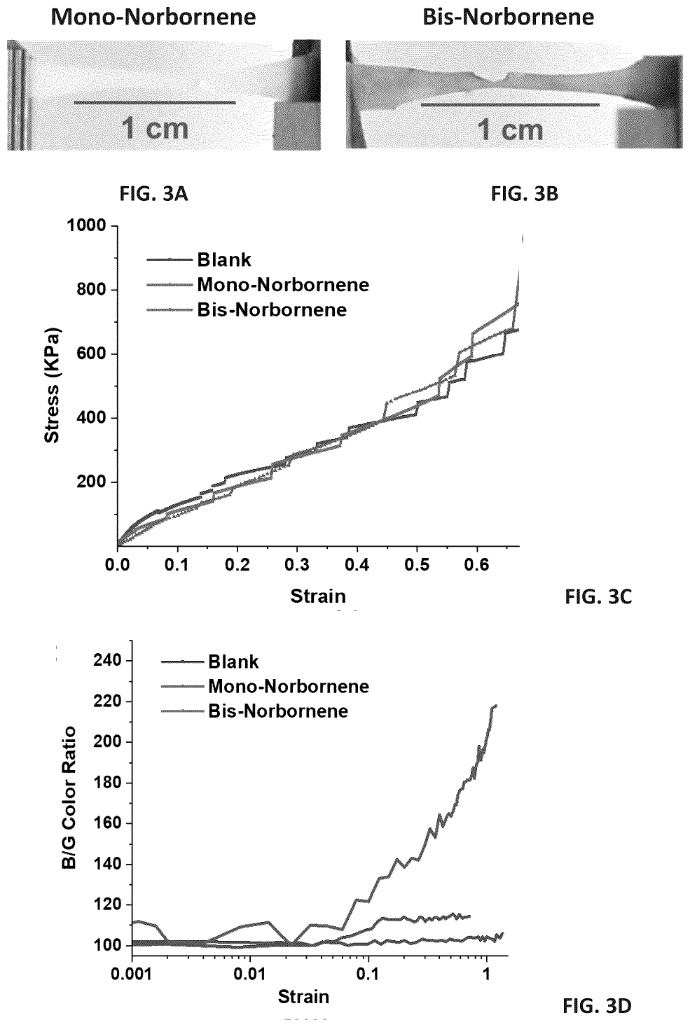

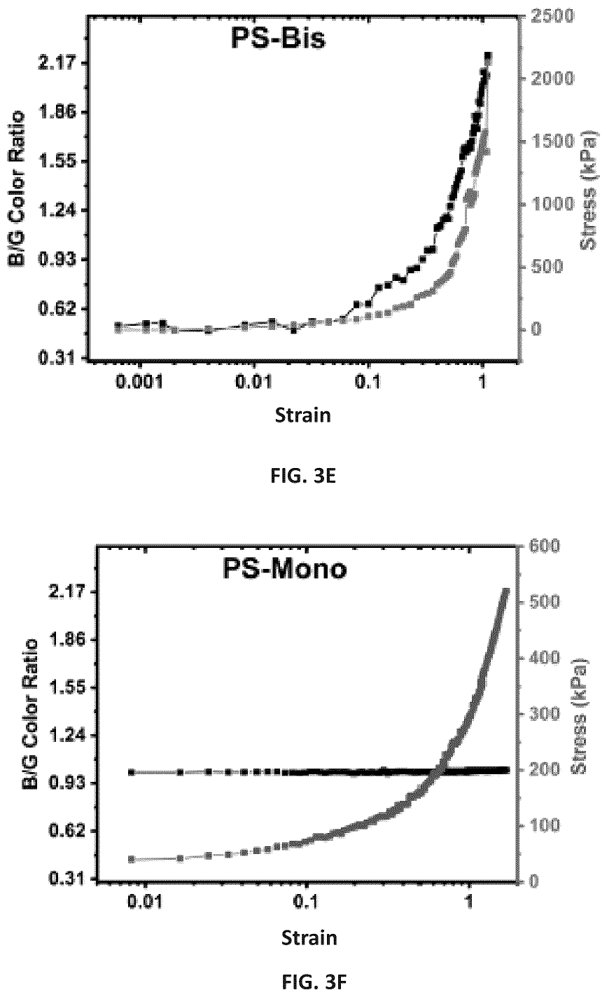

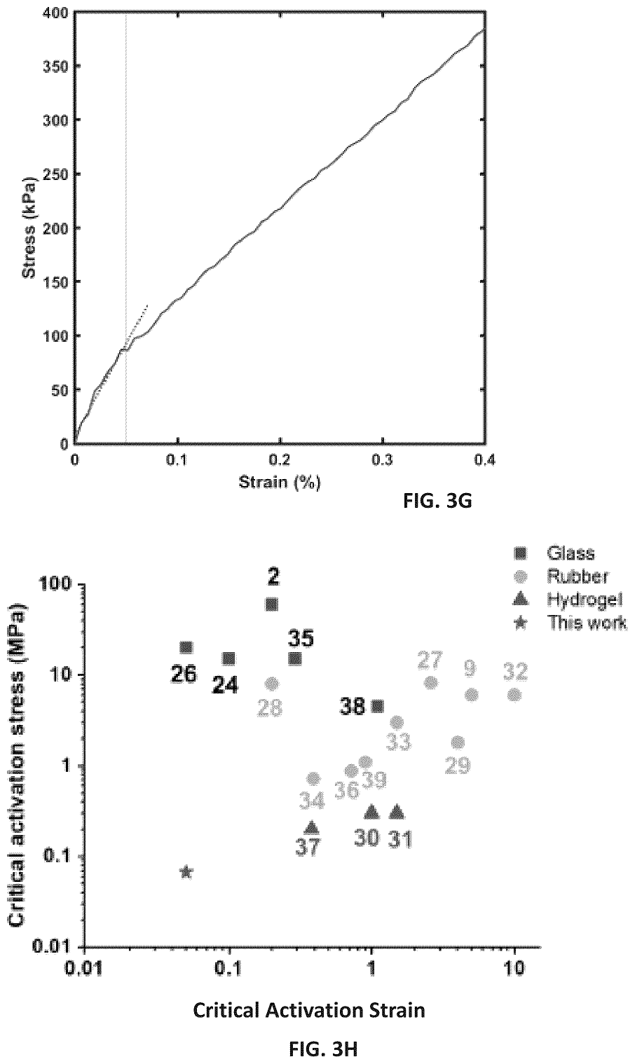

According to some of any of the embodiments described herein, upon application of force, the mechanophoric matrix features an average B/G color ratio, when determined as described herein, higher by at least 10%, or by at least 20%, or by 30%, or by 50%, or by 75%, or by 100%, or by 200%, or even higher (e.g., by 1000%), than an average B/G color ratio of an intact mechanophoric matrix. As shown, e.g., in FIG. 3D, an intact mechanophoric matrix is measured to be 100%, and upon application of force, a B/G color ratio which is higher than 100% can be measured, indicating a colorimetric change in the mechanophoric matrix.

According to some of these embodiments, the average B/G color ratio is higher by at least 30% than an average B/G color ratio of an intact mechanophoric matrix or composition-of-matter. According to some of these embodiments, the average B/G color ratio is higher by at least 50% than an average B/G color ratio of an intact mechanophoric matrix or composition-of-matter. According to some of these embodiments, the average B/G color ratio is higher by at least 75% than an average B/G color ratio of an intact mechanophoric matrix or composition-of-matter. According to some of these embodiments, the average B/G color ratio is higher by at least 100% than an average B/G color ratio of an intact mechanophoric matrix or composition-of-matter. According to some of these embodiments, the average B/G color ratio is higher by at least 150% than an average B/G color ratio of an intact mechanophoric matrix or composition-of-matter. According to some of these embodiments, the average B/G color ratio is higher by at least 200% than an average B/G color ratio of an intact mechanophoric matrix or composition-of-matter.

According to some of these embodiments, the average B/G color ratio is higher by at least 300% than an average B/G color ratio of an intact mechanophoric matrix or composition-of-matter.

According to an aspect of some of the present embodiments, there is provided a mechanophoric matrix (e.g., as described herein in any of the respective embodiments) usable in determining a presence and/or a level of a force-induced damage in a substrate (e.g., as described herein in any of the respective embodiments) or in an article-of-manufacturing comprising same. In some such embodiments, determining a presence and/or a level of a force-induced damage is effected by determining a colorimetric change.

In some such embodiments, determining a presence and/or a level of a force-induced damage is effected by determining a change in fluorescence. In some such embodiments, determining a presence and/or a level of a force-induced damage is effected by determining a change in light absorption.

In some of any of the embodiments described herein, the method is usable in detecting and/or determining a level and/or a presence of a damage in a range of applications (e.g., Department of Defense (DoD), civil infrastructure, packaging, medical device, automotive) where, e.g., composite materials and/or polymeric materials are used.

According to some of any of the embodiments described herein, the mechanophoric matrix is capable of exhibiting a force-induced change in an optical property (e.g., a colorimetric change) following force application in an amount of at least 300 Pa, or at least 400 Pa, or at least 450 Pa, or at least 500 Pa, or at least 550 Pa.

According to some of any of the embodiments described herein, the mechanophoric matrix is capable of exhibiting a force-induced change in an optical property (e.g., a colorimetric change) following force application in an amount lower than 1 MPa, or lower than 1 KPa, or lower.

According to some of any of the embodiments described herein, the mechanophoric matrix is capable of exhibiting a force-induced change in an optical property (e.g., a colorimetric change) following deformation of at least 1%, or at least 5%, or at least 10% and/or following deformation of less than 200%.

According to some of any of the embodiments described herein, the mechanophoric matrix is capable of exhibiting a force-induced change in an optical property (e.g., a colorimetric change) following deformation (e.g., strain application) of less than 200%, or less than 175%, or less than 150%, or less than 120%, or less than 100%, or less than 75%, or less than 60%, or less than 50%, or less than 40%, or even lower.

According to some of any of the embodiments described herein, the mechanophoric matrix is capable of exhibiting a force-induced change in an optical property (e.g., a colorimetric change) following deformation (e.g., strain application) that ranges from about 1 to about 200, or from about 1 to about 150, or from about 1 to about 100, or from about 1 to about 80, or from about 1 to about 60, or from about 1 to about 50, or from about 1 to about 40, or from about 1 to about 30, or from about 1 to about 20, or from about 1 to about 15, or from about 1 to about 10, or from about a to about 5, or from about 5 to about 200, or from about 5 to about 150, or from about 5 to about 100, or from about 5 to about 80, or from about 5 to about 60, or from about 5 to about 50, or from about 5 to about 40, or from about 5 to about 30, or from about 5 to about 20, or from about 5 to about 15, or from about 5 to about 10, or from about 10 to about 200, or from about 10 to about 150, or from about 10 to about 100, or from about 10 to about 80, or from about 10 to about 60, or from about 10 to about 50, or from about 10 to about 40, or from about 10 to about 30, or from about 10 to about 20, or from about 10 to about 15, %, including any intermediate values and subranges therebetween.

In some of any of the respective embodiments, metallic substrates as described herein in any of the respective embodiments are useful as piezo-resistors.

In some of any of the respective embodiments, metallic substrates as described herein in any of the respective embodiments are useful in detecting a damage in mechanically stressed articles.

In some of any of the embodiments described herein, the article-of-manufacturing is a component in a device selected from defense-related device, civil infrastructure, packaging, medical device, transportation device (e.g., vehicles, automotive, non-motor vehicle), energy harvesting device, fuel transportation device, construction elements, water treatment systems, waste treatment systems, microfluidic devices, sealing parts, textile, Articles having a corrodible surface, and agricultural device. Any additional articles-of-manufacturing or parts thereof may be considered.

As used herein throughout, the phrase “medical device” includes any material or device that is used on, in, or through a subject's body, for example, in the course of medical treatment (e.g., for a disease or injury). The subject may be human or a non-human animal, such that the phrase “medical device” encompasses veterinary devices.

In some of any of the embodiments described herein, the medical device is selected from the group consisting of a catheter, an endotracheal tube, a tampon, a tubing, a prosthetic, a medical implant, an artificial joint, an artificial valve, a needle, an intravenous access device, a cannula, a biliary stent, a nephrostomy tube, a vascular graft, an infusion pump, an adhesive patch, a suture, a fabric, a mesh, a polymeric surgical tool or instrument, an intubation device, prosthetics, artificial joints, artificial valves, adhesive patches, sutures, fabrics, a cardiovascular stent, a cardiac surgery device, an orthopedic surgery device, an orthodontic or periodontic device, a dental surgery device, a veterinary surgery device, a bone scaffold, a hemodialysis tubing or equipment, a blood exchanging device, an implantable prostheses, a bandage, a heart valve, an ophthalmic device and a breast implant.

According to some embodiments of the invention, the medical device is an implantable device.

Medical devices include, but are not limited to, medical implants (including permanent implants and transient implants), wound care devices, medical devices for drug delivery, contact lenses and body cavity and personal protection devices. The medical implants include, but are not limited to, pacemakers, heart valves, replacement joints, dialysis tubing, gastric bands, shunts, screw plates, artificial spinal disc replacements, internal implantable defibrillators, cardiac resynchronization therapy devices, implantable cardiac monitors, mitral valve ring repair devices, left ventricular assist devices (LVADs), artificial hearts, implantable infusion pumps, implantable insulin pumps, stents, implantable neurostimulators, maxillofacial implants, dental implants, catheters (e.g., indwelling catheter, urinary catheters, intravascular catheters, central venous catheter, biliary vascular catheter, pulmonary artery catheter, peripheral venous catheter, arterial line, central venous catheter, peritoneal catheter, epidural catheter, central nervous system catheter), catheter access ports, tracheal tubes, injection ports, intubation equipment, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes.

Medical devices for drug delivery include, but are not limited to, needles, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, intrauterine devices (IUDs), diaphragms and condoms.

As used herein throughout, the phrase “packaging” includes packages or containers. Such include, but are not limited to, food packages and containers, beverage packages and containers, medical device packages, packages of pharmaceutical compounds and/or kits, agricultural packages and containers (of agrochemicals), blood sample or other biological sample packages and containers, and any other packages or containers of various articles. Food packages include, but are not limited to, packages of dairy or meat products and/or containers for storage or transportation of dairy or meat products.

As used herein throughout, the phrase “energy harvesting device” includes, for example, a microelectronic device, a microelectromechanical device, a photovoltaic device and the like; As used herein throughout, the phrase “microfluidic devices” includes, for example, micro-pumps or micro valves and the like; As used herein throughout, the phrase “sealing parts” includes, for example, O rings, and the like; As used herein throughout, the phrase “agricultural devices” includes, for example, agrochemicals, systems used in agriculture; As used herein throughout, the phrase “construction elements” includes, but is not limited to, paints, walls, windows, door handles, and the like; Paints may be prepared, e.g., based on epoxy resins or polyurethanes. Epoxy-based paints are commonly used in industrial and marine environments due to their strong resistance to corrosion, abrasion, and harsh chemicals. Polyurethane paints are known for their flexibility, high gloss, and UV stability, and are widely used in automotive, aerospace, and architectural applications.

As used herein throughout, the phrase “water treatment systems” (such as for containing and/or transporting and/or treating aqueous media or water) encompasses, for example, elements, devices, containers, filters, tubes, solutions and gases and the like; and

As used herein throughout, the phrase “waste treatment systems” (such as for containing and/or disposing and/or transporting and/or treating organic waste), devices, elements, containers, filters, tubes, solutions and gases and the like.

As used herein throughout, the phrase “transportation device” includes, but is not limited to, cars, trucks, railway cars, railway tracks, bicycle components and the like.

As used herein throughout, the phrase “defense-related device” includes, but is not limited to, airframes, aircraft engines, marine vessels, sailing ship masts and the like.

Additional articles-of-manufacturing or parts thereof includes, but is not limited to, street lighting poles, oil well casings, hydroelectric turbines, nuclear reactor control rods, windows, doors, mirrors, astronomical instruments, etc. Small articles such as car engines, gears, fasteners, watches, cooking utensils, food containers, packaging, outer shells of consumer electronics, heat sinks for electronic appliances, substrates in high brightness light-emitting diode (LED) lighting, hardware tools, and many other composite and/or polymeric articles.

### General:

Herein, the phrase “linking group” describes a group (e.g., a substituent) that is attached to two or more moieties in the compound; whereas the phrase “end group” describes a group (e.g., a substituent) that is attached to a single moiety in the compound via one atom thereof.

Herein, the term “hydrocarbon” describes organic material with molecular structures containing essentially carbon and hydrogen. Hydrocarbons may Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms. The number of carbon atoms can range from 2 to 20, and is preferably lower, e.g., from 1 to 10, or from 1 to 6, or from 1 to 4. A hydrocarbon can be a linking group or an end group.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups.

Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups.

Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group.

A “cycloalkyl” group refers to a saturated on unsaturated all-carbon monocyclic or fused ring (i.e., rings which share an adjacent pair of carbon atoms) group wherein one or more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond. The cycloalkyl group can be an end group, as this phrase is defined herein, wherein it is attached to a single adjacent atom, or a linking group, as this phrase is defined herein, connecting two or more moieties.

An “aryl” group refers to an all-carbon monocyclic or fused-ring polycyclic (i.e., rings which share adjacent pairs of carbon atoms) end groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. The aryl group can be an end group, as this phrase is defined herein, wherein it is attached to a single adjacent atom, or a linking group, as this phrase is defined herein, connecting two or more moieties.

A “heteroaryl” group refers to a monocyclic or fused ring (i.e., rings which share an adjacent pair of atoms) end group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. The heteroaryl group can be an end group, as this phrase is defined herein, wherein it is attached to a single adjacent atom, or a linking group, as this phrase is defined herein, connecting two or more moieties (also referred to herein as “heteroarylene”).

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like. The heteroalicyclic group can be an end group, as this phrase is defined herein, wherein it is attached to a single adjacent atom, or a linking group, as this phrase is defined herein, connecting two or more moieties.

Herein, the terms “amine” and “amino” each refer to either a —NR′R″ group or a —N+R′R″R″′ group, wherein R′, R″ and R″′ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R″′ are hydrogen or alkyl comprising 1 to 4 carbon atoms. Optionally, R′ and R″ (and R″′, if present) are hydrogen. When substituted, the carbon atom of an R′, R″ or R″′ hydrocarbon moiety which is bound to the nitrogen atom of the amine is not substituted by oxo (unless explicitly indicated otherwise), such that R′, R″ and R″′ are not (for example) carbonyl, C-carboxy or amide, as these groups are defined herein.

An “azide” group refers to a —N═N+=N- end group.

An “alkoxy” group refers to any of an -O-alkyl, —O-alkenyl, —O-alkynyl, —O— cycloalkyl, and —O-heteroalicyclic end group, as defined herein, or to any of an -O-alkylene, —O-cycloalkyl- and —O-heteroalicyclic- linking group, as defined herein.

An “aryloxy” group refers to both an -O-aryl and an -O-heteroaryl group, as defined herein, or to an -O-arylene as defined herein.

A “hydroxy” group refers to a —OH group.

A “thiohydroxy” or “thiol” group refers to a —SH group.

A “thioalkoxy” group refers to any of an -S-alkyl, —S-alkenyl, —S-alkynyl, —S— cycloalkyl, and —S-heteroalicyclic end group, as defined herein, or to any of an -S-alkylene—S-cycloalkyl—and —S-heteroalicyclic- linking group, as defined herein.

The term “ether” describes a R′—O—R″— end group or —R′—O—R″— linking group, as these phrases are defined hereinabove, where R′ and R″ are each as defined hereinabove.

The term “thioether” describes a R′—S—R″— end group or —R′—S—R″— linking group, as these phrases are defined hereinabove, where R′ and R″ are each as defined hereinabove.

The term “aminoether” describes a R′—N-R″R″′— end group or —R′—N-R″R″′—linking group, as these phrases are defined hereinabove, where R′ and R″ are each as defined hereinabove.

A “thioaryloxy” group refers to both an —S-aryl and an —S-heteroaryl group, as defined herein, or to an —S-arylene.

A “carbonyl” or “acyl” group refers to a —C(═O)—R′ end group, where R′ is defined as hereinabove, or to a —C(═O)— linking group.

A “thiocarbonyl” group refers to a —C(═S)—R′ end group, where R′ is as defined herein, or to a —C(═S)- linking group.

A “carboxy”, “carboxyl”, “carboxylic” or “carboxylate” group refers to both “C-carboxy” and “O-carboxy” end groups, as defined herein, as well as to a carboxy linking group, as defined herein.

A “C-carboxy” group refers to a —C(═O)—O—R′ end group, where R′ is as defined herein or to a —C(═O)—O—linking group.

An “O-carboxy” group refers to an R′C(═O)—O—end group, where R′ is as defined herein, or to a —O—C(═O)— linking group.

A “carboxy linking group” refers to a —C(═O)—O—or a —O—C(═O)— linking group.

An “oxo” group refers to a═O end group.

An “imine” group refers to a═N-R′ end group, where R′ is as defined herein, or to an ═N— linking group.

An “oxime” group refers to a═N-OH end group.

A “hydrazone” group refers to a═N-NR′R″ end group, where each of R′ and R″ is as defined herein, or to a═N-NR′— linking group where R′ is as defined herein.

A “halo” group refers to fluorine, chlorine, bromine or iodine.

A “sulfinyl” group refers to an —S(═O)—R′ end group, where R′ is as defined herein, or to an —S(═O)— linking group.

A “sulfonyl” group refers to an —S(═O)₂—R′ end group, where R′ is as defined herein, or to an —S(═O)₂— linking group.

A “sulfonate” group refers to an —S(═O)₂—O—R′ end group, where R′ is as defined herein, or to an —S(═O)₂—O- linking group.

A “sulfate” group refers to an —O—S(═O)₂—O—R′ end group, where R′ is as defined as herein, or to an —O—S(═O)₂—O- linking group.

A “sulfonamide” or “sulfonamido” group encompasses both S-sulfonamido and N-sulfonamido end groups, as defined herein, as well as a sulfonamide linking group, as defined herein.

An “S-sulfonamido” group refers to a —S(═O)₂—NR′R″ end group, with each of R′ and R″ as defined herein.

An “N-sulfonamido” group refers to an R'S(═O)₂—NR″— end group, where each of R′ and R″ is as defined herein.

A “sulfonamide linking group” refers to a —S(═O)₂—NR′— linking group, where R′ is as defined herein.

A “carbamyl” group encompasses both O-carbamyl and N-carbamyl end groups, as defined herein, as well as a carbamyl linking group, as defined herein.

An “O-carbamyl” group refers to an —OC(═O)—NR′R″ end group, where each of R′ and R″ is as defined herein.

An “N-carbamyl” group refers to an R′OC(═O)—NR″— end group, where each of R′ and R″ is as defined herein.

A “carbamyl linking group” refers to a —OC(═O)—NR′— or to a —NR′—C(═O)—O—linking group, where R′ is as defined herein.

A “thiocarbamyl” group encompasses O-thiocarbamyl, S-thiocarbamyl and N-thiocarbamyl end groups, as defined herein, as well as a thiocarbamyl linking group, as defined herein.

An “O-thiocarbamyl” group refers to an —OC(═S)—NR′R″ end group, where each of R′ and R″ is as defined herein.

An “N-thiocarbamyl” group refers to an R′OC(═S)NR″— end group, where each of R′ and R″ is as defined herein.

An “S-thiocarbamyl” group refers to an —SC(═O)—NR′R″ end group, where each of R′ and R″ is as defined herein.

A “thiocarbamyl linking group” refers to a —OC(═S)—NR′— or —SC(═O)—NR′— or a —NR′—C(═S)—O—, or a —NR′—C(═O)—S—linking group, where R′ is as defined herein. An “amide” or “amido” group encompasses C-amido and N-amido end groups, as defined herein, as well as an amide linking group, as defined herein.

A “C-amido” group refers to a —C(═O)—NR′R″ end group, where each of R′ and R″ is as defined herein.

An “N-amido” group refers to an R′C(═O)—NR″— end group, where each of R′ and R″ is as defined herein.

An “amide linking group” refers to a —C(═O)—NR′— or a —NR′—C(═O)— linking group, where R′ is as defined herein.

A “urea group” refers to an —N(R′)—C(═O)—NR″R″′ end group, where each of R′, R″ and R″ is as defined herein, or an —N(R′)—C(═O)—NR″— linking group, where each of R′ and R″ is as defined herein.

A “thiourea group” refers to an —N(R′)—C(═S)—NR″R″′ end group, where each of R′, R″ and R″ is as defined herein, or an —N(R′)—C(═S)—NR″— linking group, where each of R′ and R″ is as defined herein.

A “nitro” group refers to an —NO₂ group.

A “cyano” group refers to a —C≡N group.

The term “phosphonyl” or “phosphonate” describes a -P(═O)(OR′)(OR″) group, with R′ and R″ as defined herein, or a —P(═O)(OR′)—O- linking group, with R′ as defined herein.

The term “phosphate” describes an —O—P(═O)(OR′)(OR″) end group, with each of R′ and R″ as defined herein, or an —O—P(═O)(OR′)—O- linking group, with R′ as defined herein.

The term “phosphinyl” describes a -PR′R″ end group, with each of R′ and R″ as defined herein, or a -PR′— linking group, with R′ as defined herein.

The term “hydrazine” describes a —NR′—NR″R″′ end group, where R′, R″, and R″′ are as defined herein, or to a —NR′—NR″— linking group, where R′ and R″ are as defined herein.

As used herein, the term “hydrazide” describes a —C(═O)—NR′—NR″R″′ end group, where R′, R″ and R″′ are as defined herein, or to a —C(═O)—NR′—NR″— linking group, where R′ and R″ are as defined herein.

As used herein, the term “thiohydrazide” describes a —C(═S)—NR′—NR″R″′ end group, where R′, R″ and R″′ are as defined herein, or to a —C(═S)—NR′—NR″— linking group, where R′ and R″ are as defined herein.

A “guanidinyl” group refers to an —RaNC(=NRd)—NRbRc end group, where each of Ra, Rb, Rc and Rd can be as defined herein for R′ and R″, or to an —R′NC(═NR″)—NR″′—linking group, where R′, R″ and R″′ are as defined herein.

A “guanyl” or “guanine” group refers to an R″′R″NC(═NR′)— end group, where R′, R″ and R″′ are as defined herein, or to a —R″NC(═NR′)— linking group, where R′ and R″ are as defined herein.

As used herein throughout, the term “trihaloalkyl” describes any alkyl as defined herein, that is substituted by three halo substituents, as defined herein. The halo substituents can be on any carbon of the alkyl, optionally on one of the carbons of the alkyl, for example, on a terminal carbon atom of the alkyl. The three halo substituents can be the same or different, preferably the same. A non-limiting example for a trihaloalkyl is trifluoromethyl (—CF₃).

As used herein throughout, the term “trihaloalkyl” describes any alkyl as defined herein, that is substituted by three halo substituents, as defined herein. The halo substituents can be on any carbon of the alkyl, optionally on one of the carbons of the alkyl, for example, on a terminal carbon atom of the alkyl. The three halo substituents can be the same or different, preferably the same. A non-limiting example for a trihaloalkyl is trifluoromethyl (—CF₃).

The term “sulfoxide” or “sulfinyl” describes a —S(═O)R′ end group or an —S(═O)— linking group, as these phrases are defined hereinabove, where R′ is as defined hereinabove.

The term “vinyl sulfide” describes a R'S-CH═CH₃ end group or a —R'S-CH═CH₂-linking group, as these phrases are defined hereinabove, where R′ is as defined hereinabove.

For any of the embodiments described herein, the compound described herein may be in a form of a salt, for example, an acid addition salt or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention.

Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials:

The synthesis of the exemplary mechanophoric compounds, bis-norbornene spiropyran (Compound 8) and mono-norbornene spiropyran (Compound 7) and of the exemplary crosslinker, nonane-1,9-diyl bis(bicyclo[2.2.1]hept-5-ene-2-carboxylate) (CL), involved several chemicals obtained from different suppliers.

For the 5-norbornene-2-carboxylic acid, a mixture of 8:2 endo- and exo- isomers (obtained from Alfa Aesar®) was used, along with methyl isopropyl ketone (MIPK; obtained from Sigma-Aldrich®), ethanol (EtOH; obtained from Bio-lab), hydrobromic acid (HBr; obtained from Fisher Chemical), triethylamine (TEA, obtained from Sigma-Aldrich®), tetrahydrofuran (THF; obtained from Bio-lab), 4-dimethylaminopyridine (DMAP; obtained from Aaron Chemical), triethylamine (Et₃N; obtained from Alfa Aesar®), methanol (obtained from Bio-lab), ethyl acetate (obtained from Sigma-Aldrich®), NaHCO₃ (obtained from Sigma-Aldrich®), NaCl (obtained from Sigma-Aldrich®), Na₂SO₄ (obtained from Sigma-Aldrich®), dichloromethane (obtained from Sigma-Aldrich®), ethylether (obtained from Sigma-Aldrich®), acetone (obtained from Sigma-Aldrich®), acetonitrile (CH₃CN; obtained from Sigma-Aldrich®), hexane (obtained from Sigma-Aldrich®), 1-Ethyl-3-(3-dimethylaminopropyl)carbodiimide (EDCI-HCl; obtained from Sigma-Aldrich®) among others.

Syntheses of Compounds 1-4 (FIG. 1A) and norbornene anhydride were performed according to known procedures [Potisek et al. (2007) supra; Davis et al. (2009) supra; Kim et al. Macromolecules 51, 9177-9183 (2018); Simpson et al. Macromolecules 41, 3613-3619 (2008)].

For the preparation of the spiropyran alginate, sodium alginate (180 kDa) (obtained from KIMICA Corporation, Japan), 1-ethyl-3-(3-dimethylaminopropyl)-carbodiimide hydrochloride (EDC; Sigma-Aldrich®), N-hydroxysuccinimide (NHS; obtained from Sigma-Aldrich®), 2-(N-morpholino) ethanesulfonic acid (MES; obtained from Glentham Life Sciences), (4-(1,2,4,5-Tetrazin-3-yl)phenyl)methanamine hydrochloride (Tz, obtained from Sigma-Aldrich®), dialysis tube (12-14 kDa MWCO, D9527 obtained from Sigma-Aldrich®), hydroxylamine (obtained from Sigma-Aldrich®) were used.

For the preparation of the spiropyran-acrylamide alginate film, all chemicals were obtained from Sigma-Aldrich® (Israel).

For the synthesis of styrene-vinyl benzyl chloride copolymer (PS-Cl), vinylbenzyl chloride (VBC; obtained from Sigma-Aldrich®), styrene (St; obtained from Alfa Aesar®), KOH (Bio-Lab) and benzoyl peroxide (BPO; obtained from Merck®) were used.

Monomers were purified before use by passing them through basic aluminum oxide (Acros Organics) to remove stabilizing agents.

For the preparation of styrene-vinyl benzyl chloride copolymer functionalized with azide (PS-N₃), sodium azide (Sigma-Aldrich®) and N, N-dimethylmethanamide (DMF; Bio-Lab) were used.

Benzyl azide was prepared following known procedures [Trejo-Maldonado et al., React. Funct. Polym. 2021, 164, 104919]. CuCl was washed with acetic acid and dried under vacuum prior to use. KOH (Bio-Lab) and benzoyl peroxide (BPO, Merck) were used are received.

For the preparation of the spiropyran wool, alpaca wool (obtained from alpaca farm, Israel), NaOH (Bio-Lab), (3-Mercaptopropyl) methyldimethoxysilane (MPTES; obtained from Sigma-Aldrich®), 2-Hydroxy-2-methylpropiophenone (obtained from Sigma-Aldrich®), 5,5-dithio-bis-(2-nitrobenzoic acid) (obtained from Holland Moran) were used.

All reagents for synthesis were purchased from commercial sources and used as received unless otherwise mentioned. Water (deionized water) were purified through a Millipore system.

Equipment: UV photoreaction equipped with 2 UV lamps (Philips™ TUV 8W/G8T5) with a power rating of 8 W and a dominant wavelength of 254 nm. pH readings: pH readings were taken using an AZ 86505 pH/mV/Cond./TDS/Temperature meter with a SIN: 1104961.

Digital image: The digital images and video were obtained using a Canon™ DS126291 camera with a Canon Macro Lens EF-S 60 mm 1: 2.8 USM lens under ambient room light conditions.

NMR measurements: All ¹H and ¹³C NMR spectra were recorded using an AVANCE U 400 MHz Bruker™ spectrometer.

Mass Spectrometer measurement: High-resolution mass spectrometry was performed in a Waters™ LCT Premier Mass Spectrometer (ESI) or a Bruker™ maxis impact with APCI solid probe.

ATR-FTIR measurement: Attenuated total reflection-Fourier transform infrared (ATR-FTIR) spectra were recorded on a Thermo Scientific Nicolet FTIR iS50 spectrometer at room temperature in the 400-4000 cm⁻¹ range. FTIR spectroscopy was carried out with powder samples pelleted after careful grinding with anhydrous 200 mg KBr (99+%, FTIR grade, Sigma-Aldrich®) and compared with freshly prepared blank KBr pellets.

UV-Vis measurement: UV-Vis measurements were done in Thermo Evolution™ 220 spectrophotometer after diluting the sample in a solvent.

Fluorescence Image analysis: Images of fluorescence variation of the mechanochromic material were acquired on a fluorescence microscope (Nikon ECLIPSE Ts2R, Japan) using a 4× phase contrast objective (CFI Super Plan Fluor ELWD ADM, China) equipped with a high-resolution digital camera from Hamamatsu (ORCA-Spark Digital CMOS camera C11440-36U). The fluorescence image was acquired with a laser excitation of 460 nm, and the emission was observed with a 575 nm RFP filter. All the microscope images were processed and analyzed using Fiji (an image processing software by ImageJ) or NIS-Elements AR 5.41.01 software (an image processing software by Nikon™).

GPC: Gel permeation chromatography (GPC) analysis was done in THF at 30° C., according to the polymer's solubility, using a Thermo LC system. The THF system consisted of one Tosoh TSKgel HHR-L guard column and four TSKgel G4000HHR columns in sequence, working at a 1 mL/minute flow rate.

Tensile tests: Tensile tests were performed using a dynamic mechanical analyzer (Q800 Dynamic Mechanical Analyzer (DMA), TA Instruments) with a tensile speed of 400 μm/minute. The samples were cut into dog-bone shapes with a gauge length of 30 mm, a width of 3 mm, and a thickness of 1 mm. The cut gel specimens were exposed to white light for about 5 minutes before the test to erase the purple color along the edges due to the force-induced SP-MC transition.

Folding: The bis-norbornene spiropyran-acrylamide film was folded between glass slides at varying angles. Two pieces of glass were used to sandwich a portion of the film and secure it, and another two pieces of glass were used to sandwich and fix the remaining portion of the film. The film was folded inward along the gap between the glass pieces at various angles. The exemplary bis-norbornene spiropyran-functionalized tetrazine alginate film was similarly folded (FIGS. 8A-E).

Calculation of molar ratio of the monomeric units in random copolymers PS-Cl: molar ratio of the monomeric units F=[St]/[VBC] was calculated based on ¹H NMR spectra according to the following equation [Willdorf-Cohen et al., 2023, supra]:

$$F = \frac{( {I}_{c} - 2 \times {I}_{n} ) / 5}{{I}_{n} / 2}$$

where Ia is the integral value of the signals at 6.1-7.2 ppm attributed to the aromatic protons, and In is the integral value of the signal at 4.0-4.3 ppm attributed to the protons of chloromethylene group (—CH₂Cl) in the VBC unit. Which is:

$$F = \frac{( 10.43 - 2 \times 2 ) / 5}{2 / 2} = 1.286$$

$$\{ \begin{matrix}\frac{[ St ]}{[ VBC ]} = F \\ [ St ] + [ VBC ] = 1\end{matrix}$$

$$\{ \begin{matrix}\frac{[ St ]}{[ VBC ]} = 1.286 \\ [ St ] + [ VBC ] = 1\end{matrix}$$

$$[ St ] = 1 - 0.44 = 0.56$$

$$[ VBC ] = 0.44$$

where [St] and [VBC] are molar parts of St 56% and VBC 44% units in a copolymer, respectively.

Calculation of mechanophore decoration in mono- and bis- norbornene spiropyran: The mechanophore decoration was estimated based on the amount of norbornene consumed according to the following equations:

### PS-Bis:

Spiropyran functional amount is the same as crosslink density in PS-Bis.

$$\frac{mol of crosslinks}{mol of reactive sites \times 100 \%} = \frac{mol of norbornene groups \times 2}{mol of reactive sites \times 100 \%} = \frac{0.0168 \times {10}^{- 3} \times 2}{0.19 : ( 0.44 \times 159 + 0.56 \times 104 ) \times 0.44 \times 100 \%} = 5.136 \%$$

Crosslink density in mol %:

$$\frac{g of crosslinks}{g of polymer \times 100 \%} = \frac{10 \times {10}^{- 3}}{0.19 \times 100 \%} = 0.52 \%$$

### PS-Mono:

Spiropyran functional amount in weight (wt.) %:

$$\frac{g of spiropyran}{g of polymer \times 100 \%} = \frac{8 \times {10}^{- 3}}{0.19 \times 100 \%} = 0.41 \%$$

### Crosslink Density in Mol %:

$$\frac{mol of crosslinks}{mol of reactive sites \times 100 \%} = \frac{mol of norbornene groups \times 2}{mol of reactive sites \times 100 \%} = \frac{0.0168 \times {10}^{- 3} \times 2}{0.19 : ( 0.44 \times 159 + 0.56 \times 104 ) \times 0.44 \times 100 \%} = 5.136 \%$$

### Crosslink Density in Weight %.

$$\frac{g of crosslinks}{g of polymer \times 100 \%} = \frac{6.7 \times {10}^{- 3}}{0.19 \times 100 \%} = 0.35 \%$$

Wt. % represents % by weight of a total weight.

Calculation of mechanophore decoration in Alginate: According to FIGS. 6A-B and comparative ¹H NMR spectra (data not shown), the degree of tetrazine substitution of the available carboxylic acid groups of alginate is 2.82%. 3 mg/mL ALG-Tetrazine containing 0.4255*10-3 mM/ml tetrazine. Based on ¹H NMR spectra (not shown), it is assume that the crosslink density in mol is 100%. Crosslink density in wt. % is therefore as follows:

$$\frac{g of crosslinks}{g of polymer \times 100 \%} = \frac{0.4255 \times {10}^{- 6} + 2 \times 594}{( 0.003 + Wsp ) \times 100 \%} = \frac{0.126 \times {10}^{- 3}}{( 0.126 \times {10}^{- 3} + 0.003 )} = 4 \%$$

Determination of thiol (SH) groups in wool: The amount of thiol group in the wool was determined throughout the functional process by soaking the wool overnight in a solution of Ellman's reagent and measuring its absorbance at a wavelength of 412 nm. 0.8 mM, 0.4 mM, 0.2 mM, and 0.1 mM standard MPTES solutions were prepared in a buffer solution containing 0.1 M sodium phosphate and 1 mM EDTA in pH 8.0. 4 mg/mL 5,5-dithio-bis-(2-nitrobenzoic acid) in a buffer solution to Ellman solution were prepared. The 0.25 mL MPTES, 2.5 mL buffer solution, and 50 μL Ellman solution were mixed, and incubated at room temperature for 15 minutes. The UV-vis spectra was used to take a full absorbance spectrum of the compound.

Pressing experiment of bis-norbornene spiropyran-functionalized wool: Bis-spiropyran-functionalized wool (0.486 grams) was uniaxially pressed with 0-890 MPa in a steel die (diameter: 12.8 mm) using a manual uniaxial press (HOLZMANN-MASCHINEN 4170 Haslach, AUSTRIA), their pressure is read by a Pressure Gauge RAM DIA45m for 10 Ton Hydraulic Shop Press.

Finite element analysis (FEA) model: The finite element model was designed to determine the stresses a rectangular film of bis-norbornene spiropyran-acrylamide alginate film (“ALG-Bis alginate-acrylamide film”, “alginate film”) undergoes while incrementally folded from a flat configuration to a fully folded one. This stress is then correlated with the activation or not of the spiropyran molecules and thus, determines the folding energy needed to activate them.

Experimentally, a 10×5×0.5 mm³ alginate film was attached to two glass slides and they were folded incrementally, one on the other, at different angle values measured by a protractor. The slides left a gap of 3 mm to allow free movement of the film during the folding (FIG. 8F). To reduce computation time, the problem was simplified to a 5×5×0.5 mm³ square with a symmetry plane (data not shown), as the geometry is symmetrical to the folding plane [Moerman, M. and Gibbon, K, JOSS 2018, 3(22), 506].

During the quasi-static simulations, lateral and top surfaces were free of displacement constrain, and boundary conditions were applied on one edge of the model corresponding to the folding plane and the bottom face corresponding to the displacement of the glass slide. The symmetry plane composed of one fixed node at the edge, a sliding line at the plane's base, and symmetric nodes on the rest of the plane.

A displacement loading was applied to the nodes corresponding to the interface between the glass slide and the film, i.e., all the nodes were distant of 1.5 mm from the symmetry plane. After translating the center of rotation at y=Y+e, where e is the thickness of the film; Y the initial y coordinates; and y the y coordinates after translation, the following displacement load were applied:

$$\{ yn = - xn for θ = 90$$

$$yn = xn ( cos θ - 1 ) for θ < 90$$

To determine the Von Mises stress and standard energy density (SED) required to activate mechanophores, a finite element model of the folding was developed using the opensource FE Pardiso solver FEBio version 4.2 [Moerman and Gibbon (2018), supra] via the GIBBON open-source toolbox version 3.5.0 [Maas et al., J. Biomech. Eng. 2012, 134 (1), 011005] used with MATLAB 9.14 R2023a (The Mathworks Inc., Natick, MA, USA) for pre and post-processing of the simulations. The runtime of each simulation was in the range of 2 minutes for small folding to 20 minutes for the fully folded model using a 64-bit Windows™ 10 pc with an Intel(R) Xeon(R) W-2245 CPU®3.90 GHz and 128 GB RAM.

Mechanical properties of the ALG-Bis alginate-acrylamide film used have been determined by using a 3345 Universal testing machine (Instron, Norwood MA, USA) mounted with a 2530-5N load (Instron, Norwood MA, USA) and 2711 micro grips (Instron, Norwood MA, USA). 12*10*0.5 mm³ samples were extended at a rate of 10 mm/minute.

Young's modulus of the film was determined by fitting the strain-stress curve up to 30% strain with a linear model using the fit function of Matlab™. E=248 kPa was found (FIG. 8G). The Poisson's ratio was fixed at 0.49 as the films were assumed to be incompressible.

The meshing of the model into quadratic hexahedral (hex20) elements was performed using the meshing tool provided by FEBio. A convergence study has been done before running all the simulations to find the minimal amount of elements and, thus, the shortest simulation time, leading to a constant solution. The parameter used in the simulation was the distance between the nodes. Only a few values of distances between nodes allowed to have an entire number of elements both in x, y and z directions, as presented in Table A.

TABLE A

| Distance | Number of | Number of | Total |
|---|---|---|---|
| between | elements | elements | number of |
| nodes | over x, y | over z | elements |
| 0.5 | 10 | 1 | 100 |
| 0.25 | 20 | 2 | 800 |
| 0.125 | 40 | 4 | 6400 |
| 0.1 | 50 | 5 | 12500 |
| 0.0625 | 80 | 8 | 51200 |
| 0.05 | 100 | 10 | 100000 |

FIG. 8H shows the values of Effective strain (Von Mises stress) and calculation time over the number of elements for a folding angle of 100°. It showed that from 6400 elements, the solution differs only by 2.5% from the best value we got and that for 12500 elements, the solution differs only by 1.6%. However, the simulation time exponentially increases with the number of elements increasing from a few hundred seconds (346 seconds) at 12500 elements to close to one hour (3347 seconds) for 51200 elements without a significant increase in the accuracy of the simulation. This is why simulations were ran with 12500 elements.

The stress is the average Von Mises stress on all the thicknesses of the gel obtained by the following equation [Salengon, J. Handbook of Continuum Mechanics; Springer Berlin Heidelberg: Berlin, Heidelberg, 2001]:

$$vonMiseskP α \frac{1}{n} {\sum }^{√} \frac{1}{2} ( {( {s}_{1} - {s}_{2} )}^{2} + ( {( {s}_{2} - {s}_{3} )}^{2} + ( {( {s}_{3} - {s}_{1} )}^{2} ) \cdot 1000$$

Where S1—S3 are the principal stresses.

### Example 1

### Design and Chemical Syntheses

The present inventors have conceived utilizing the advantages of Click chemistry for conjugating mechanophores to different substrates, without the complicated requirements of multistep chemical processes.

In order to enable Click addition of a spiropyran with polymers, two exemplary novel mechanophore derivatives were designed and prepared using exemplary synthetic pathways, as shown in FIG. 1A.

Spiropyran activation can be initiated by UV-light irradiation and low pH. In order to ensure that the activation of spiropyran was caused predominately due to intramolecular forces within polymer chains, two novel exemplary mechanophoric compounds were synthesized: one with a single norbornene end group (mono-norbornene, Compound 7), and another with two end groups (bis-norbornene, Compound 8). While activation of Compound 8 and de-activation of Compound 7 would indicate a force-induced activation of the spiropyran, the activation of both Compound 7 and Compound 8 would indicate a non-force induced activation. Compound 7 therefore serves as a negative control in the following experiments.

Positions 5′ and 8′ on the indole of the norbornene were selected as attachment points as they showed the greatest sensitivity to force activation [Grossweiler et al. J. Am.

Chem. Soc. 2015, 137, 19, 6148-6151; Barbee et al. J. Am. Chem. Soc. 2018, 140 (40), 12746-12750].

Exemplary synthetic approaches for the preparation of Compounds 7 and 8 (mono- and bis- norbornene, respectively) are as described in FIG. 1A, and as follows.

### Synthesis of 2,3,3-trimethyl-3H-indol-5-yl bicyclo[2.2.1]hept-5-ene-2-carboxylate (5)

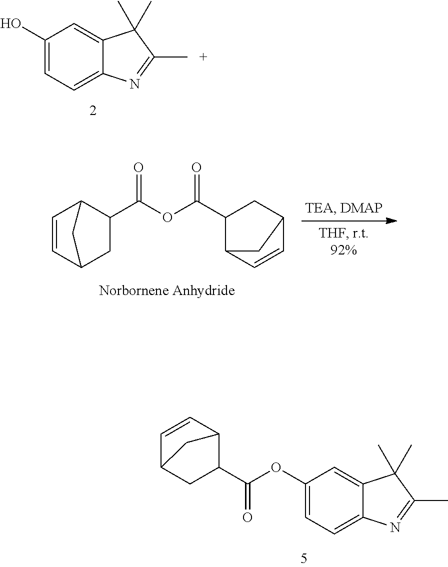

Compound 2 (600 mg, 3.42 mmol, 1.00 mol equivalent) and triethylamine (0.50 mL, 3.59 mmol, 1.05 mol equivalent) were dissolved in tetrahydrofuran (31 mL). The mixture was dropwise added with norbornene anhydride (1.23 gram, 4.77 mmol, 1.40 mol equivalent) and 4-dimethylaminopyridine (62 mg, 0.51 mmol, 0.15 mol equivalent) in tetrahydrofuran (5 mL) at 0° C. After the mixture was stirred at room temperature overnight, the reaction was quenched with methanol (1 mL) and diluted with ethyl acetate. The organic phase was washed with a saturated NaHCO₃ solution and NaCl several times then dried with anhydrous Na₂SO₄. After filtration, the product was purified by silica gel column chromatography using 30:1 dichloromethane/methanol as eluent. The final purified product 5 (460 mg, 1.55 mmol, 92%) was obtained as a yellow viscous liquid. The chemical structure was verified by ¹H NMR (400 MHz, CDCl₃) and ¹³C NMR (101 MHz, 5 CDCl₃). A 20:80 ratio between the exo- and endo-configurational isomers of norbornene acid, a starting material in the preparation of Compound 5 (FIG. 1A), was determined by comparing the integral intensity signal, while comparing to the known shifts of each isomer (Liu et al., ACS Macro Lett. 2019, 8, 846).

¹H NMR (400 MHz, CDCl₃-d_{I}, δ): 7.49 (t, J=8.3 Hz, 1H), 7.04-6.98 (m, 1H), 6.97-6.90 (m, 1H), 6.30-6.04 (m, 2H), 3.45-3.28 (s, 0.44H), 3.27-3.16 (m, 1H), 3.05-2.92 (m, 1H), 2.55-2.42 (m, 0.45H), 2.26 (d, J=3.2 Hz, 3H), 2.11-2.05 (m, 0.45H), 2.03-1.97 (m, 0.54H, Hi-exo), 1.58-1.33 (m, 3H), 1.30 (d, J=4.9 Hz, 6H); ¹³C NMR (126 MHz, CDCl₃, 6): 198.25, 174.63, 172.41, 152.55, 144.04, 140.06, 138.63, 136.62, 132.85, 123.02, 118.04, 115.85, 53.67, 46.53, 41.67, 35.06, 30.71, 22.54, 14.82.

### Synthesis of 5-((bicyclo[2.2.1]hept-5-ene-2-carbonyl)oxy)-1,2,3,3-tetramethyl-3H-indolium iodide (6)

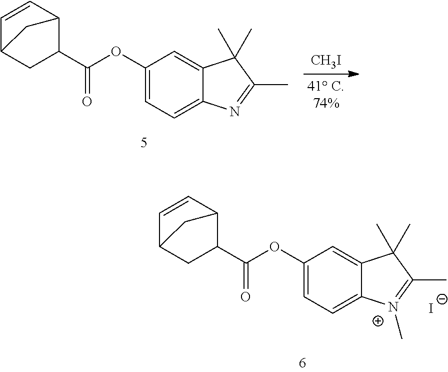

A solution of Compound 5 (136 mg, 0.46 mmol, 1.00 mol equivalent) in methyl iodide (0.44 mL, 6.90 mmol, 15.0 mol equivalent) was stirred at 41° C. for 24 hours. The mixture was filtered and washed with cold ethyl ether. The residue was dissolved in a small amount of acetone and then dropwise added to a cold rapidly stirred ethyl ether solution resulting precipitate. Compound 6 (149 mg, 0.34 mmol, 74%) was obtained by filtration as a white powder. The chemical structure was verified by ¹H NMR (400 MHz, CDCl₃) and ¹³C NMR (101 MHz, CDCl₃).

¹H NMR (400 MHz, CDCl₃-dl, 6): 7.76 (t, J=8.6 Hz, 1H), 7.33-7.26 (m, 1H), 7.25-7.19 (m, 1H), 6.29-6.02 (m, 2H), 4.20 (dd, J=3.7, 1.0 Hz, 3H), 3.37 (s, 0.43H), 3.29-3.16 (m, 1H), 3.04 (dd, J=5.1, 1.0 Hz, 3H), 2.99 (s, J=3.5 Hz, 1H), 2.49 (ddd, J=9.0, 4.4, 1.4 Hz, 0.54H), 2.02 (dddd, J=11.7, 7.1, 3.7, 2.5 Hz, 1H), 1.71 (s, 0.88H), 1.62 (d, J=4.5 Hz, 6H), 1.58-1.42 (m, 3H); ¹³C NMR (101 MHz, CDCl₃-dl, 6): 198.95, 174.63, 171.71, 152.97, 144.33, 140.61, 138.63, 136.02, 131.77, 123.98, 118.04, 116.63, 53.07, 46.53, 43.08, 41.67, 36.23, 30.71, 22.95, 14.46.

### Synthesis of (±)-8-hydroxy-1,3′,3′-trimethyl-6-nitrospiro[chromene-2,2′—indolin]-5′-yl bicyclo[2.2.1]hept-5-ene-2-carboxylate (7)

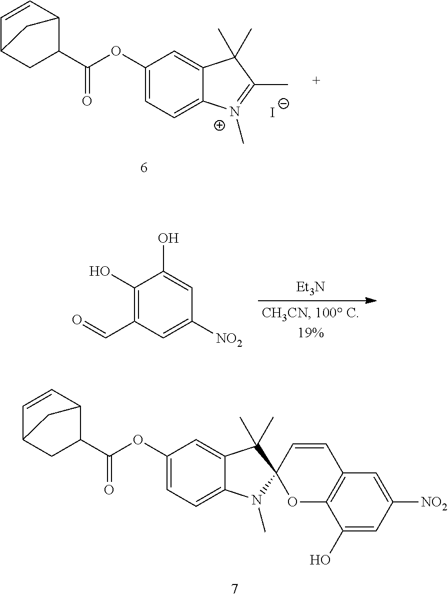 [SMILES: "Et3N" ; CC1=[N+](C)c2ccc(OC(=O)C3CC4C=CC3C4)cc2C1(C)C ; [I-] ; O=Cc1cc([N+](=O)[O-])cc(O)c1O ; CN1c2ccc(OC(=O)C3CC4C=CC3C4)cc2C(C)(C)[C@]12C=Cc1cc([N+](=O)[O-])cc(O)c1O2]

Compound 6 (437 mg, 1.00 mmol, 1 mol equivalent), 2,3-dihydroxy-5-nitrobenzaldehyde (183 mg, 1.00 mmol, 1 mol equivalent) and triethylamine (0.28 mL, 2.00 mmol, 2 mol equivalent) were dissolved in acetonitrile (9.5 mL) and reflux at 100° C. for 5 hours. Then the product was purified by silica gel column chromatography using 50:1 dichloromethane/methanol as eluent to obtain Compound 7 (90.1 mg, 0.19 mmol, 19%) as a black solid. The chemical structure was verified by ¹H NMR (400 MHz, CDCl₃). The chemical structure was verified by ¹H NMR (400 MHz, CDCl₃), ¹³C NMR (101 MHz, CDCl₃) and HRMS.

¹H NMR (400 MHz, CDCl₃-d_{I}, S): 7.69 (ddd, J=18.8, 2.6, 1.7 Hz, 2H), 7.06-6.67 (m, 3H), 6.53 (t, J=8.5 Hz, 1H), 6.37-6.05 (m, 2H), 5.92-5.76 (m, 1H), 3.41 (s, 0.48H), 3.29-3.17 (m, 1H), 3.02 (s, 1H), 2.76 (d, J=4.6 Hz, 3H), 2.50 (ddd, J=8.9, 4.5, 1.6 Hz, 0.54 H), 2.13-1.99 (m, 1H), 1.82 (d, J=5.3 Hz, 0.79H), 1.64 (d, J=8.5 Hz, 1H), 1.51-1.36 (m, 1.39H), 1.26 (dd, J=25.4, 4.7 Hz, 6H); ¹³C NMR (101 MHz, CDCl₃-d_{I}, δ): 174.30, 170.51, 151.33, 145.07, 140.04, 138.18, 135.75, 132.21, 128.40, 119.88, 118.70, 116.05, 106.84, 51.97, 49.80, 49.40, 46.00, 45.29, 43.61, 42.73, 41.79, 31.63, 29.44, 28.89, 22.32, 19.59, 15.42; HRMS (ESI) m/z: [M+H⁺] calculated for C₂₇H₂₇N₂O₆⁺, 475.1869; found 475.1876.

### Synthesis of (±)-1′,3′,3′-trimethyl-6-nitrospiro[chromene-2,2′-indoline]-5′,8-diyl bis(bicyclo[2.2.1]hept-5-ene-2-carboxylate) (8)

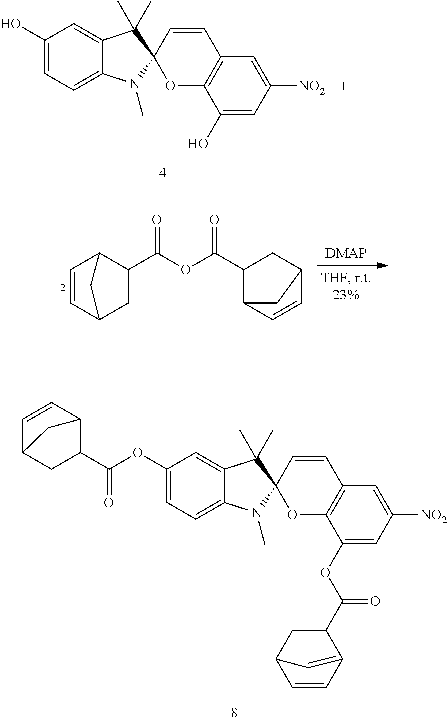

Compound 4 (300 mg, 0.84 mmol, 1.0 mol equivalent) and 4-dimethylaminopyridine (187 mg, 1.52 mmol, 1.8 mol equivalent) were fully dissolved into THF (7.5 ml), followed by the dropwise addition of norbornene anhydride (564 mg, 2.18 mmol, 2.6 mol equivalent). After stirring for 7 hours at room temperature, the reaction was quenched with methanol (1 ml). The crude product was purified by column chromatography eluting with 50:1 dichloromethane/methanol. After removing the solvent, the product was purified by recrystallization from boiling hexane to afford Compound 8 (124 mg, 0.20 mmol, 23%) as a shallow green powder. The chemical structure was verified by ¹H NMR (400 MHz, CDCl₃), ¹³C NMR (101 MHz, CDCl₃) and HRMS.

¹H NMR (400 MHz, CDCl₃-d_{I}, δ): 8.07-7.63 (m, 2H), 7.13-6.67 (m, 3H), 6.47 (td, J=8.5, 4.8 Hz, 1H), 6.31-5.93 (m, 3.38H), 5.88 (ddd, J=10.3, 4.8, 2.2 Hz, 1H), 5.62 (ddd, J=41.1, 5.8, 3.0 Hz, 0.6 H), 3.45-2.70 (m, 3H), 2.54-2.39 (m, 0.4H), 2.17 (s, 0.4H), 2.11-1.94 (m, 1H), 1.88-1.58 (m, 1H), 1.53-1.27 (m, 5H), 1.23-1.09 (m, 6H), 1.08-0.94 (m, 0.76H); ¹³C NMR (101 MHz, CDCl₃-d_{I}, δ): 173.53, 172.20,151.43,145.07,140.04,138.18, 137.43, 135.75, 132.21, 128.40, 121.28, 120.06, 119.03, 115.48, 106.89, 51.97, 49.80, 49.40, 46.85, 46.44, 46.00, 44.93, 43.62, 42.73, 42.34, 41.79, 40.10, 31.63, 30.63, 29.51, 28.89, 25.89, 22.69, 19.62, 14.17; HRMS (ESI) m/z: [M+H⁺] calculated for C₃₅H₃₅N₂O₇+, 595.2439; found 595.2439.

### Example 2

### Mechanophore-Conjugated Synthetic Polymers

Mechanical activation was demonstrated using styrene-vinyl benzyl chloride copolymer (PS-Cl) as an exemplary synthetic polymer, and azide was used as an exemplary moiety (e.g., a clickable moiety) that can undergo an addition reaction (e.g., Click addition), to provide an exemplary synthetic polymer substrate, PS-N₃. An exemplary synthetic procedure for the preparation of PS-N₃ is as follows.

### Synthesis of styrene-vinyl benzyl chloride copolymer (PS-Cl)

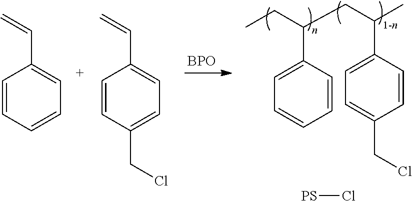

Copolymer PS-Cl was synthesized according to Willdorf-Cohen et al. [ACS Appl. Energy Mater. 2023, 6, 2, 1085-1092], using conventional radical polymerization with a 1:0.25 styrene to VBC molar ratio, as follows.

Benzoyl peroxide (BPO) (0.64 gram, 2.5% of monomer, 2.48 mmol), VBC (2.8 mL, 19.8 mmol), and styrene (9.1 mL, 79.42 mmol) were added to a Schlenk flask and sealed with a rubber septum. The polymerization was conducted under Argon atmosphere at 123° C. with magnetic stirring until the solution became viscous and the magnetic stirrer stopped moving. Then, the reaction mixture was dissolved in CH₂Cl₂ (DCM) and the polymer PS-Cl precipitated in methanol to provide a pure white powder (10 grams, 88% yield). The chemical structure was verified by ¹H NMR (CDCl₃), and GPC was used to determine a molecular weight (Mₙ) of 30 kDa (see, FIG. 2A).

¹H NMR (CDCl₃) δ 6.14-7.21 (10H, Ph-H), 4.4-4.8 (2H, —CH₂Cl), 1.2-2.2 (3H, —CHCH₂—). Mn (THF-SEC): 30 KDa, D=2.8.

### Synthesis of an exemplary clickable moiety, styrene-vinyl benzyl chloride copolymer functionalized with azide (PS-N₃)

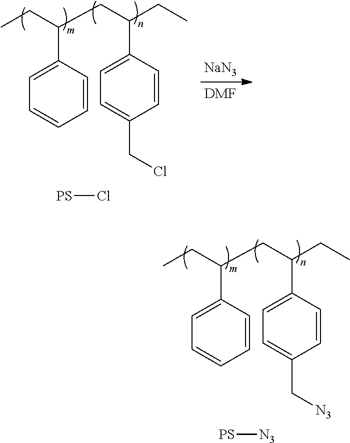

PS-Cl (Mn=30 kDa, 500 mg) and sodium azide (1023 mg, 15.7 mmol) were dissolved in DMF (10 mL) and stirred at room temperature for 48 hours. The mixture was precipitated with cold water 2 times and dry in the chemical hood to provide PS-N₃ as a white powder (335 mg, 67% yield). The chemical structure was verified by ¹H NMR (400 MHz, CDCl₃), and GPC was used to determine a molecular weight (Mₙ) of 36 kDa (see, FIG. 2B).

### Synthesis of nonane-1,9-diyl bis(bicyclo[2.2.1hept-5-ene-2-carboxylate) (endo/exo=3/1) (Compound 9; CL)

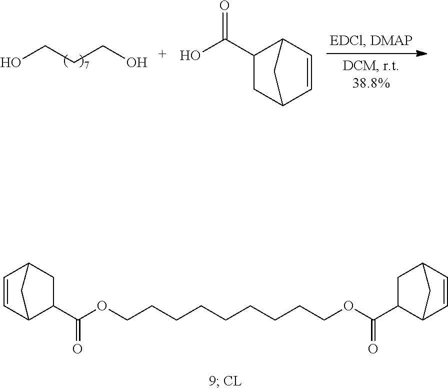

In a Schlenk flask, Nonane-1,9-diol (1 gram, 6.24 mmol) norbornene acid (3.45 gram, 24.96 mmol), DMAP (0.3 gram, 2.5 mmol) were dissolved in anhydrous DCM (120 mL). Then EDCI HCL (4.8 gram, 24.96 mmol) was added in several portions into the mixture which has been cooled down to 0° C. Then, the reaction was left to stir at room temperature for 24 hours. The crude product was purified by column chromatography eluting with 3:1 Hexane/DCM. Upon solvent evaporation, CL was afforded (970 mg, 2.42 mmol, 39%) as a transparent solution. The chemical structure was verified by ¹H NMR (400 MHz, CDCl₃), ¹³C NMR (101 MHz, CDCl₃) and HRMS.

¹H NMR (400 MHz, CDCl₃-d_{I}, δ): 6.21-5.87 (m, 4H), 4.15-3.88 (m, 4H), 3.23-2.87 (m, 5.35 H), 2.25-2.17 (dd, J=10.3, 4.4 Hz, 0.46 H), 1.95-1.85 (tdd, J=12.1, 7.9, 3.9 Hz, 2H), 1.67-1.49 (m, 5.45 H), 1.45-1.40 (m, 2.86 H), 1.39-1.23 (m, 12H); ¹³C NMR (101 MHz, CDCl₃-d_{I}, δ): 176.35, 174.86, 138.05, 137.75, 135.80, 132.38, 64.57, 64.32, 49.64, 46.64, 46.38, 45.74, 43.38, 43.23, 42.55, 41.65, 30.33, 29.41, 29.39, 29.19, 29.16, 28.70, 28.68, 25.94; HRIMS (ESI) m z: [M+H⁺] calculated for C₂₅H₃₇04+, 401.2686; found 401.2689.

### Preparation of polystyrene-functionalized bis-norbornene spiropyran (PS-Bis) or mono-norbornene spiropyran (PS-Mono)

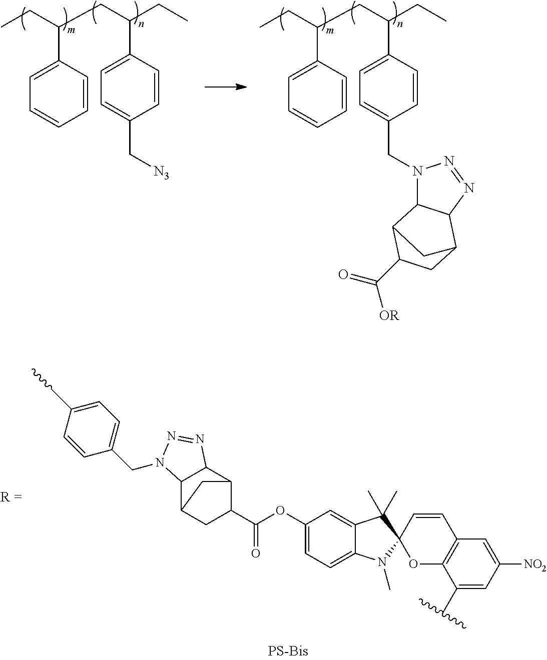

(Dashed lines represent attachment points as R on one end and to another PS-N₃ PS unit on the other end)

To prepare PS-Bis, PS-N₃ (190 mg) dissolved in dichloromethane (DCM; 10 mL), added with bis-norbornene spiropyran (Compound 8; 10 mg, 0.0168 mM) then heat to 50° C. for 3 days.

Zhang et al. [Macromol. Rapid Commun. 2016, 37 (16), 1311-1317] previously reported that an azide-norbornene polyaddition which proceeded at room temperature took 14 days to gelate, and over 3 months in total for the azide groups to be fully consumed.

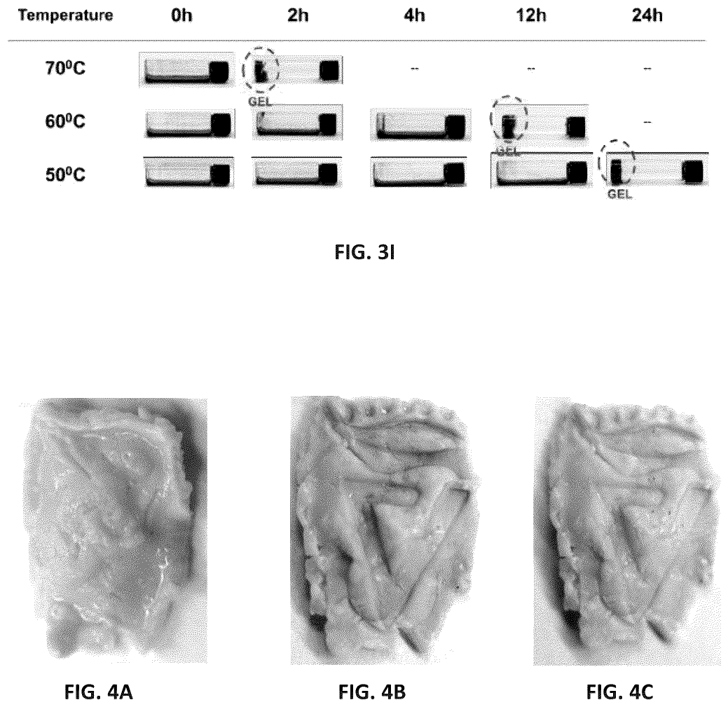

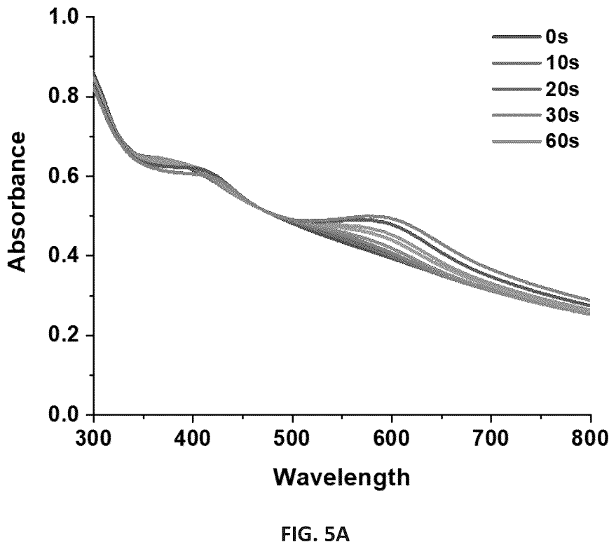

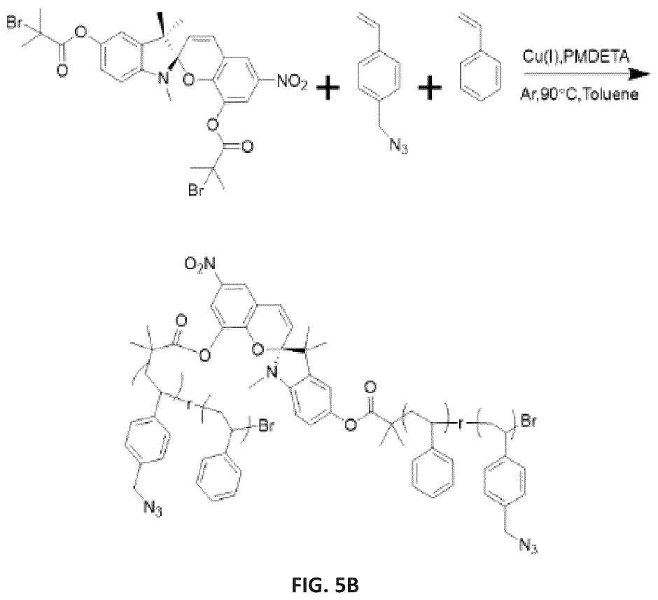

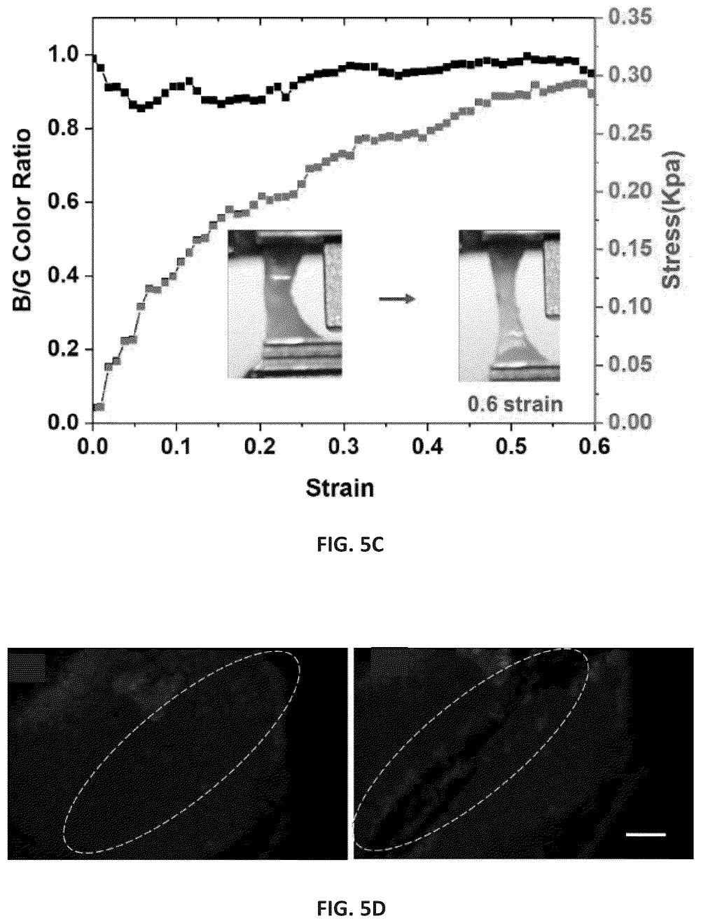

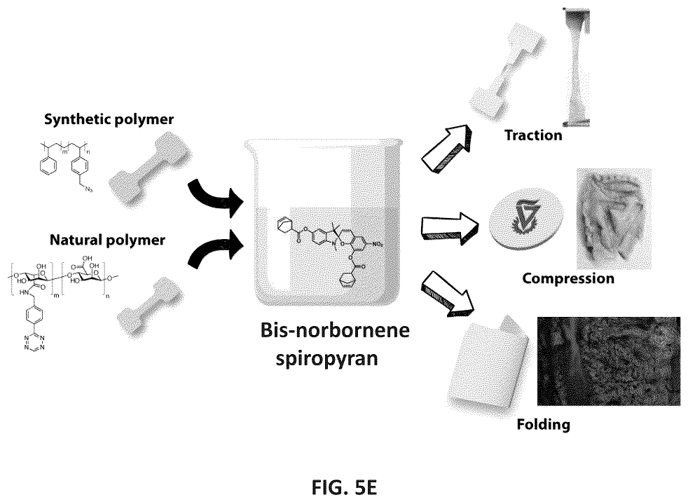

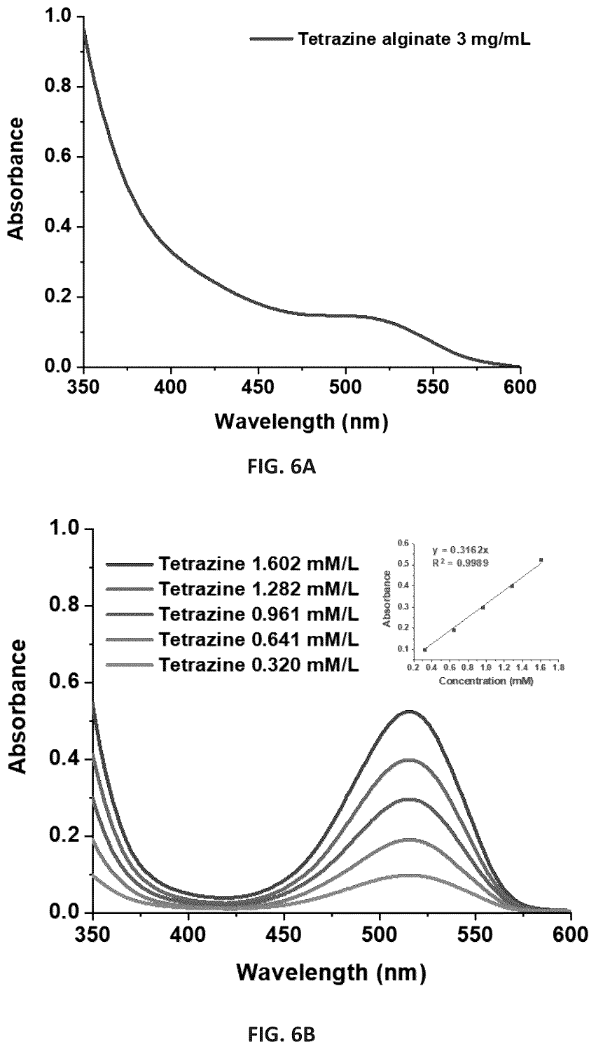

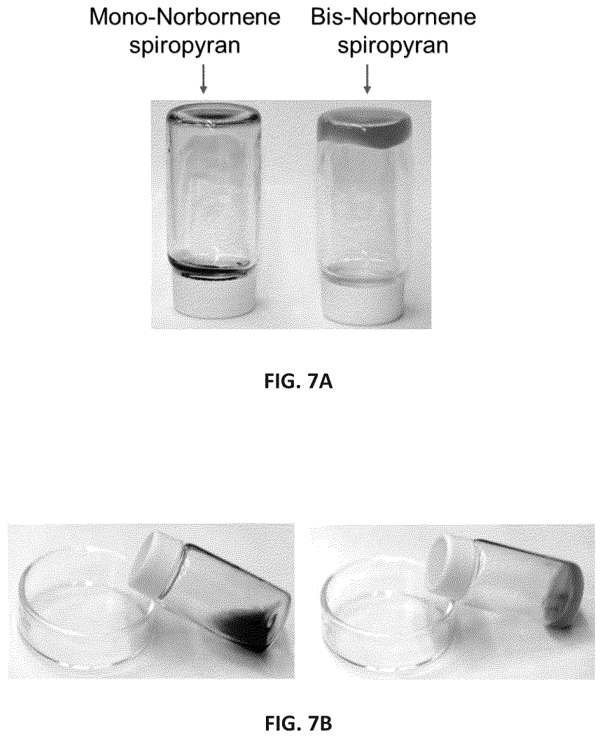

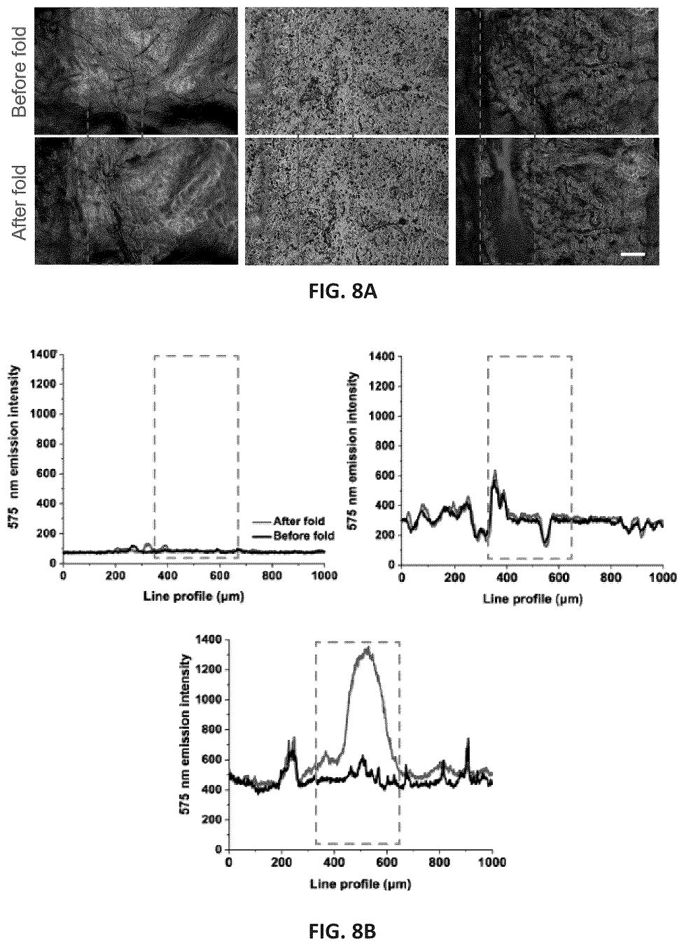

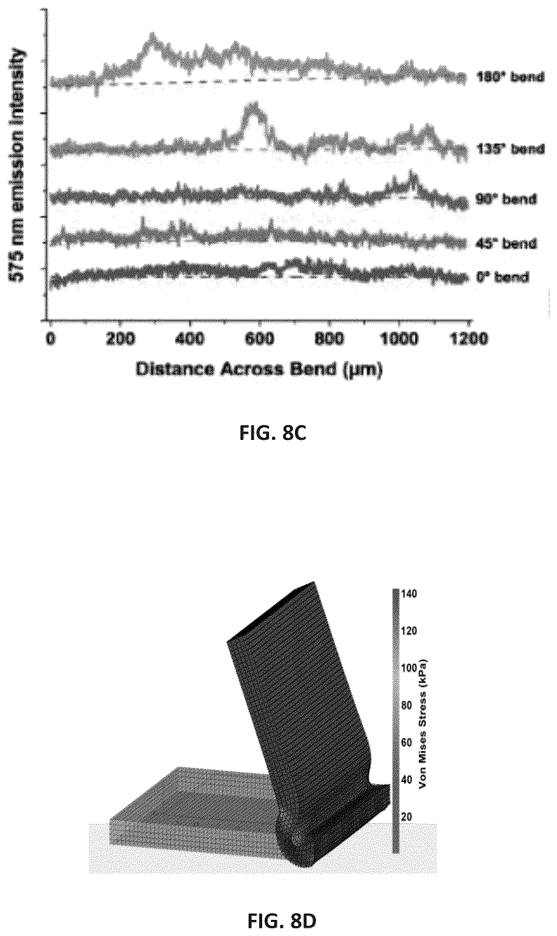

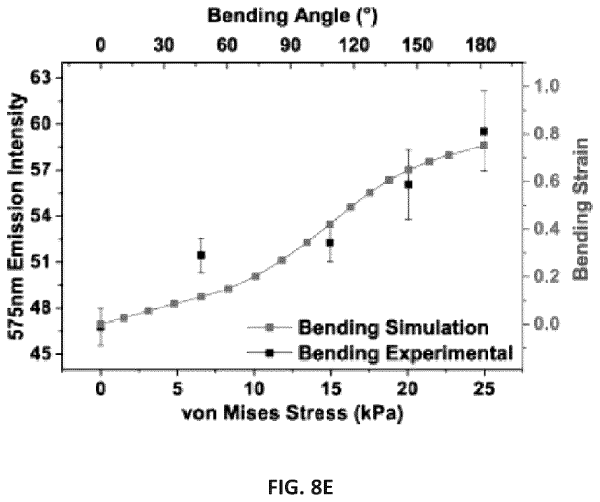

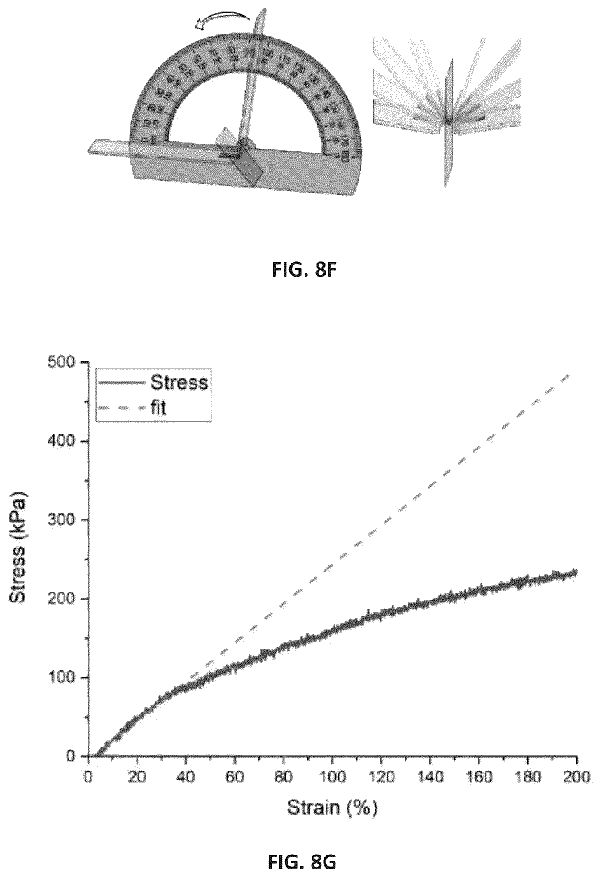

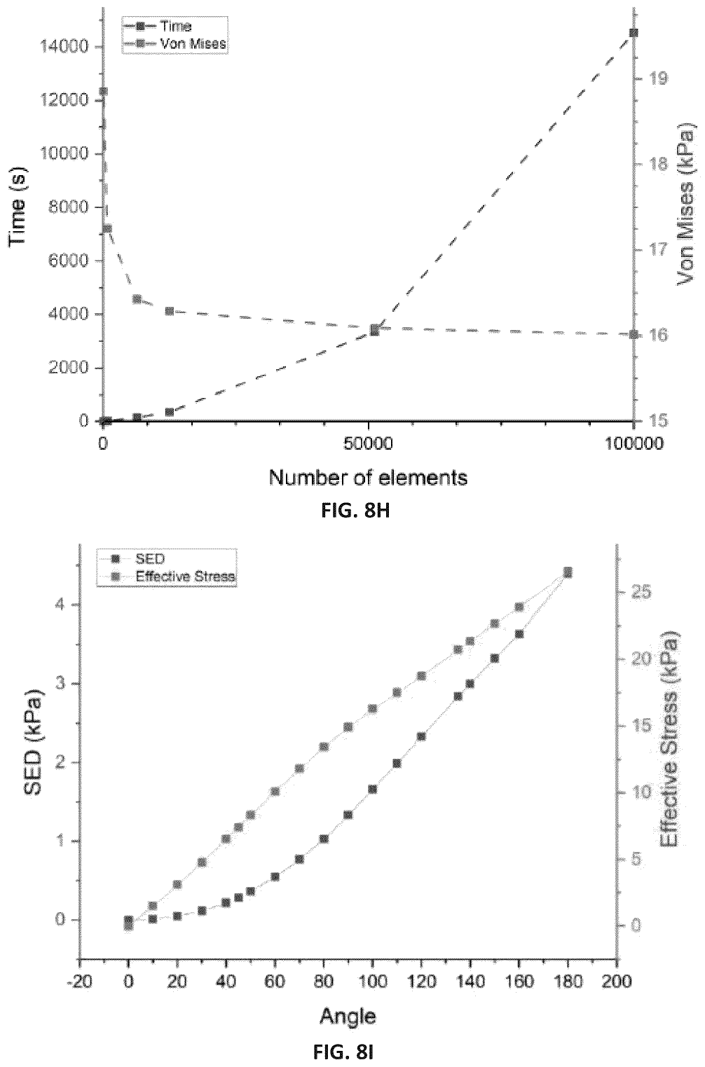

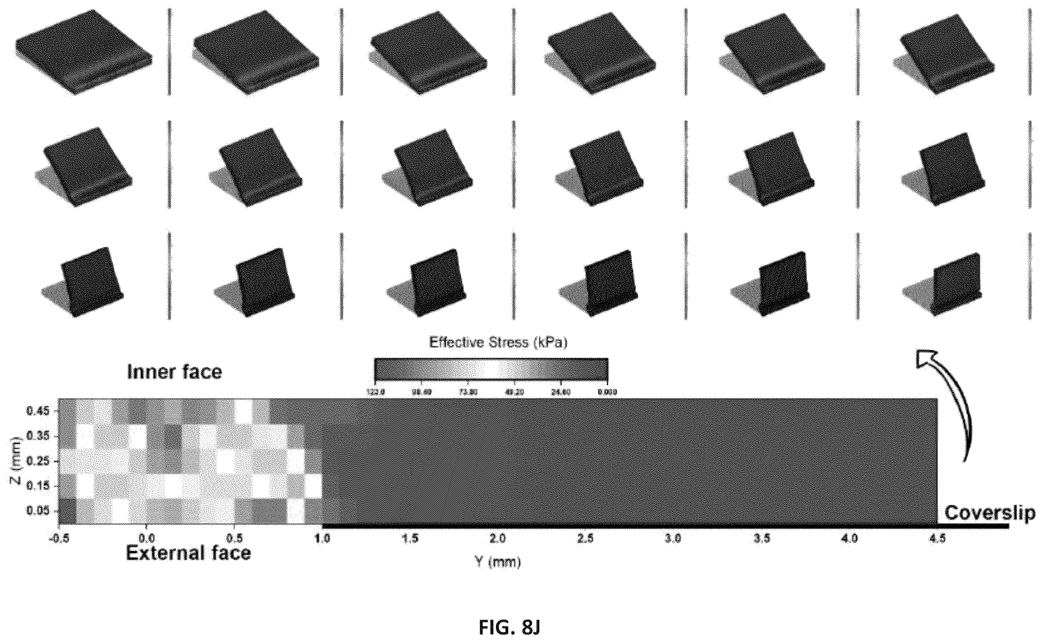

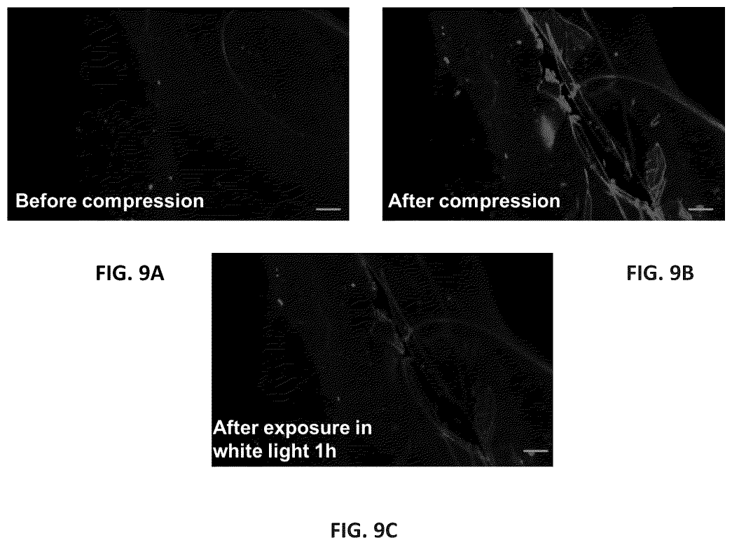

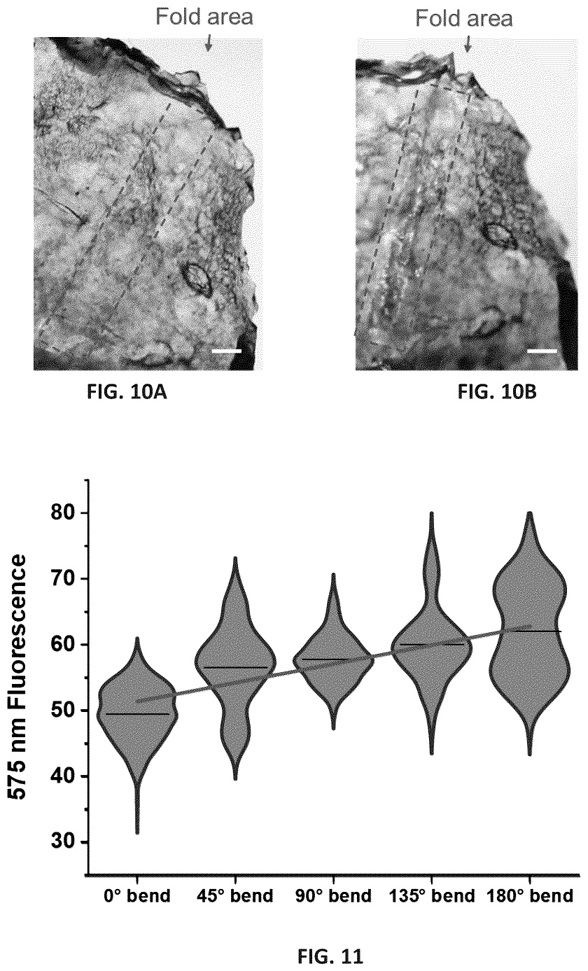

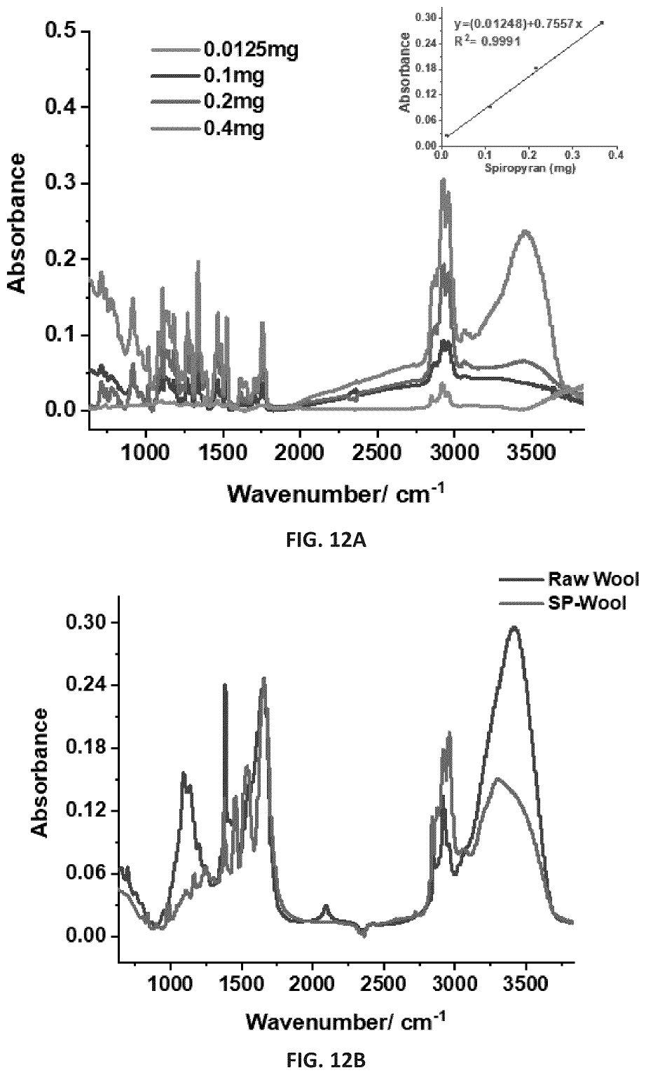

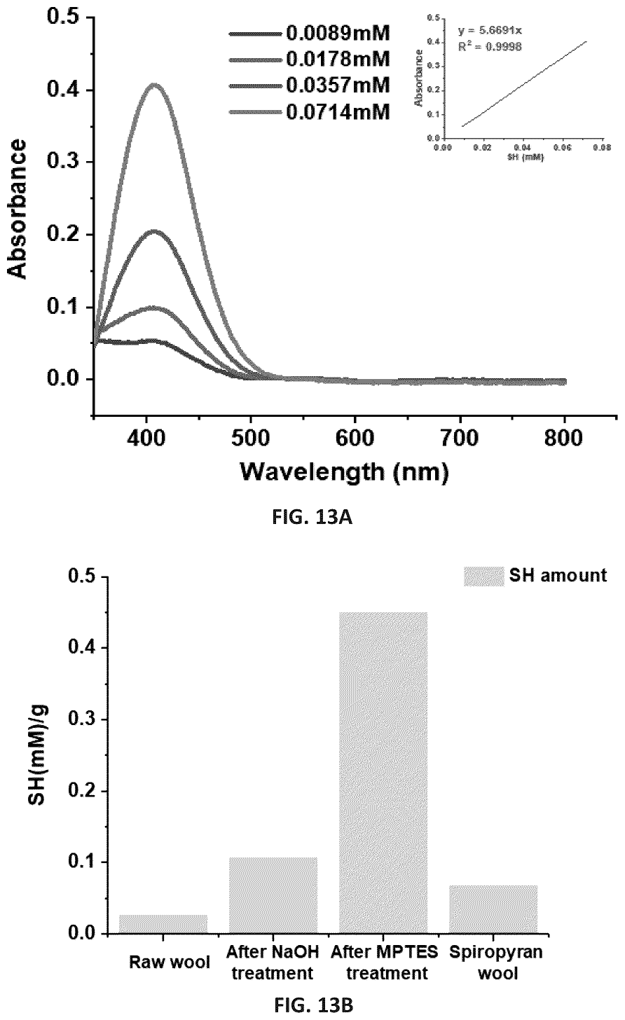

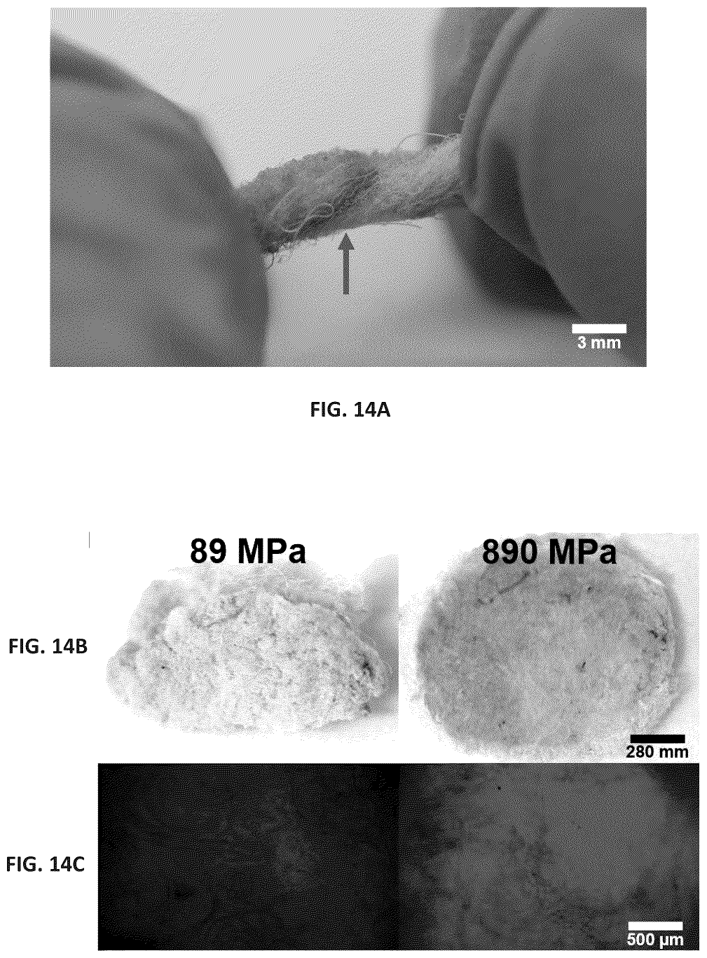

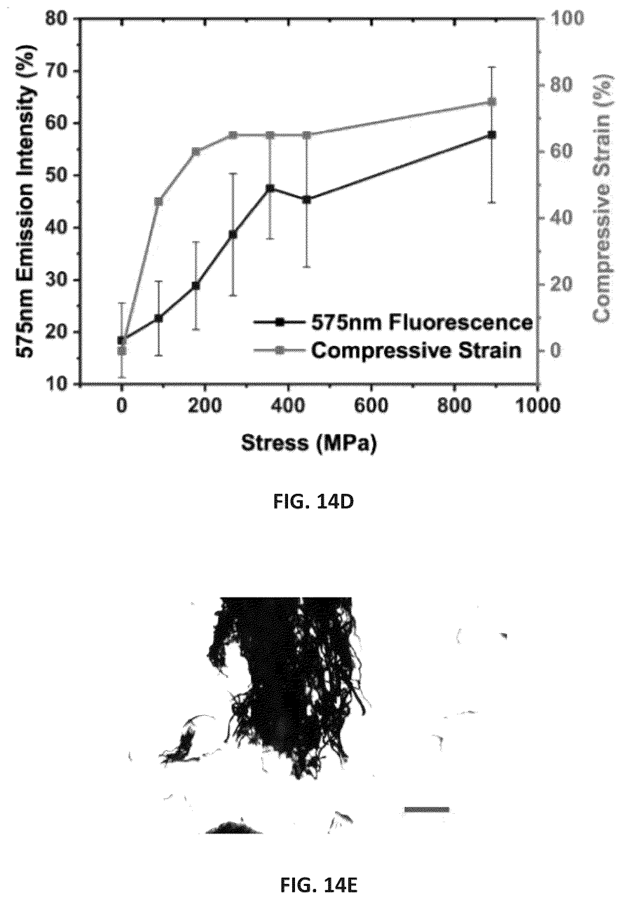

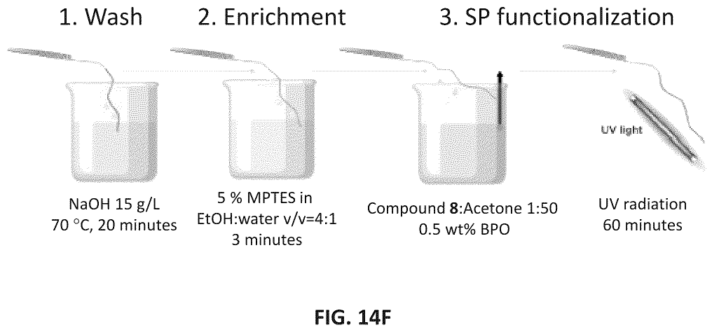

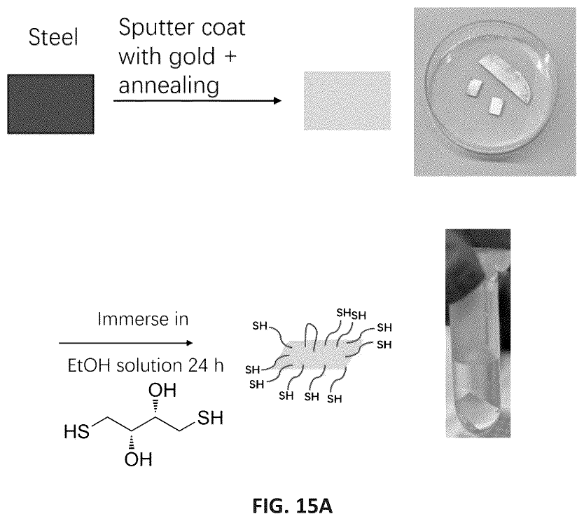

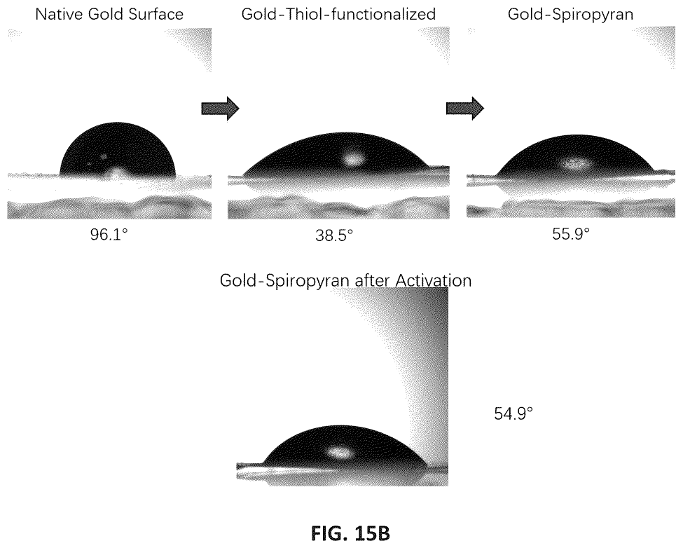

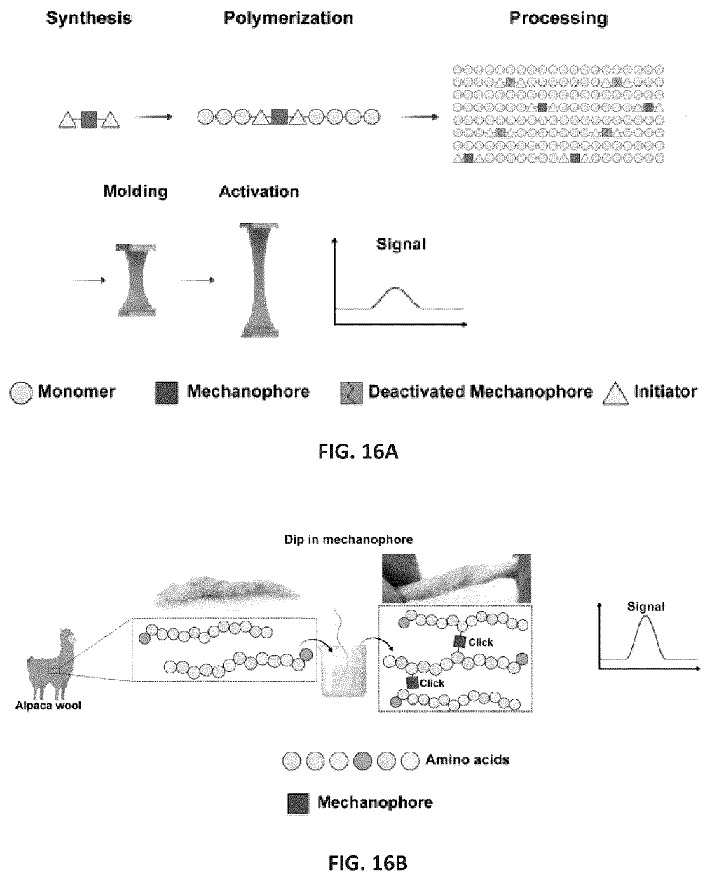

Heating is necessary for the azide-norbornene Click Reaction to take place at reasonable time scales. The reaction was first performed at temperatures of 50° C., 60° C., and 70° C., and the gelation was photographed during a span of 24 hours. The photographs, presented in FIG. 31, indicate gelation begins after about 2 hours at 70° C., after 12 hours at 60° C., and after 24 hours at 50° C.

To prepare PS-Mono, PS-N₃ (190 mg) dissolved in DCM (10 mL). To this solution, mono-norbornene spiropyran (Compound 7; 8 mg, 0.0168 mM) and the nonane-1,9-diyl bis(bicyclo[2.2.1]hept-5-ene-2-carboxylate) (an exemplary crosslinker; Compound 9; CL; 6.7 mg, 0.0168 mM) was added (in order to compensate for the cross-linking effected by the bis-norbornene spiropyran). The resulting mixture was then heated to 50° C. for 3 days. The chemical structure was verified by ¹H NMR (400 MHz, CDCl₃), and the composition of the random copolymer PS-Cl was calculated from the ¹H NMR spectra [Agami et al., ACS Appl. Polym. Mater. 2022, 4, 12, 9250-9256], and the molar percentage of VBC in the copolymer PS-Cl was determined to be 28.9%.

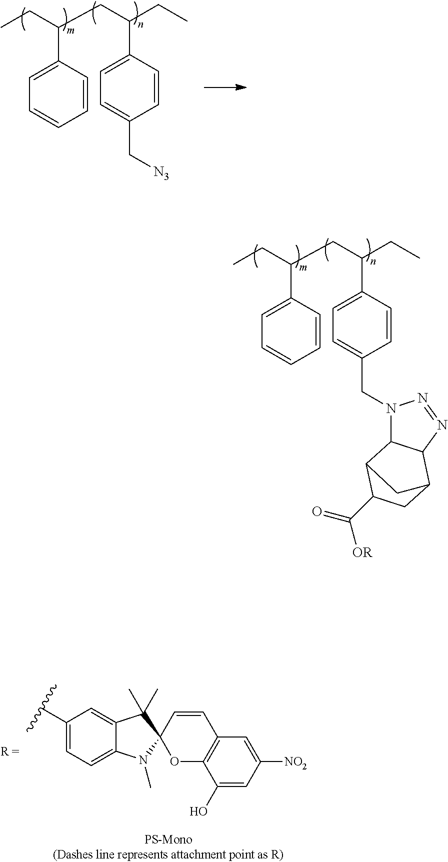

The effect of mono- and bis- norbornene spiropyran-based mechanophores on the force-activation of a polymer was tested. For this purpose, tension was measured on the exemplary mono- and bis- norbornene spiropyran-functionalized polystyrene, where dog-bone structures were cut from thin films, exposed to white light for 10 minutes to ensure conversion of the spiropyran to its un-activated form, and then mounted on a DMA machine with a constant strain rate of 1 mm/s. Then, stress and B/G value were measured as a function of strain with the purple color characterized by the ratio of blue to green channels for the dog-bone specimen.

RGB values were measured based on a video from a DSLR camera equipped with a macro lens. The data was run through Image Analysis software to remove the background and the color channels were separated and compared. B/G color ratio was calculated from isolated dog-bone image, and the ratio of the blue color intensities of the samples divided by the green color intensities was determined in order to ascertain the presence of purple color (CITE).

Dog-bone specimens of mono- and bis- norbornene spiropyran-based mechanophores were each loaded onto a dynamic mechanical analysis (DMA) machine under a constant load rate of 0.2600 N/minute from 0 to 2.000 N. Photographs are presented in FIGS. 3A and 3B, respectively, and the respective data are plotted in FIGS. 3C-D in comparison with a PS-N₃ (“Blank”).

These data indicate that the exemplary bis-norbornene spiropyran-functionalized polystyrene provided an exceedingly favorable outcome following force activation, as it demonstrated an increased B/G value and a distinct colorimetric change (FIGS. 3B and 3D-F). This change in B/G signal indicates the conversion of spiropyran to merocyanine [Davis et al., (2009) supra; Qian et al. Chem (2021), 7 (4), 1080-1091], and represents activation of the exemplary mechanophore.

Surprisingly, the change in B/G signal reflects inherent competition between energy dissipation and was already detected at 5% strain (FIGS. 3E and 3G), which is an order of magnitude more sensitive than most other rubbery or even glassy polymers where spiropyran had previously been incorporated. See, FIG. 3H, wherein each number denotes a specific reference, as follows:

Glass: 2-Vidavsky et al. 2019, supra; 24-Beiermann et al. J. Mater. Chem. 2011, 21 (23), 8443; 26-Kim et al. (2018) supra; 35-Jia et al. Macromolecules (2019), 52 (20), 7920-7928; 38-Huo et al. Macromolecules (2023, 56 (5), 1845-1854.

Rubber: 9-Zhang et al. 2014, supra; 27—Li et al. Polymer (2016), 99, 521-528; 28—Lee et al. J. Am. Chem. Soc. (2010), 132 (45), 16107-16111; 29-Cao, Z. Macromol. Chem. Phys. (2020), 221 (15), 2000190; 32-Song et al. Polymer (2022), 250, 124878; 33 Chen et al. Chem. Sci. (2021), 12 (33), 11098-11108; 34-Qiu et al. Macromolecules (2020), 53 (10), 4090-4098; 36-Kim et al. Macromolecules (2018), 51 (22), 9177-9183; 39—Lin et al. J. Am. Chem. Soc. (2018), 140 (46), 15969-15975.

Hydrogel: 30-Chen et al. Adv. Mater. (2017), 29 (21), 1606900; 31-Xu et al. Gels (2022), 8 (4), 208; 37-Zhang et al. J. Mater. Chem. C (2018), 6 (43), 11536-11551.

Often, in bulk materials, chain scission may dominate and increase the stress on neighboring strands, which lowers the potential for force to propagate through the mechanophore [Lloyd et al. J. Am. Chem. Soc. (2023), 145(2), 751-768].

Without being bound by any particular theory, in this case where the spiropyran is acting as a cross-linker, the competition with premature network failure may be diminished, leading to more uniform force distribution. The mechanical nature of the spiropyran activation in the exemplary PS-Bis is further supported by negligible B/G color ratio changes were seen in the PS-N₃ and PS-Mono samples, even at strains exceeding 100% (see, e.g., FIGS. 3A, 3D and 3F).

The effect of compressing the exemplary bis-norbornene spiropyran-functionalized polystyrene was visually tested. As can be seen in FIGS. 4A-C, pressing the polymer with a stamp resulted in a clear change in color in the pressed regions of the polymer. Following a 5-minute exposure to a white light source, the color of the pressed regions of the polymer dissipated.

FIG. 5A shows the change in absorbance of the exemplary mono-norbornene spiropyran-functionalized polystyrene following exposure to a 16 W ultraviolet light (254 nm) over time. It can be seen that upon exposure to a UV light source, the absorbance of the exemplary mono-norbornene spiropyran-functionalized polystyrene at 590 nm (λₘₐₓ) quickly dissipates.

Without being bound to any particular theory, it is assumed that due to the very short distance between norbornenes and the absence of any spacers between the norbornene and spiropyran moieties, this biases the reactivity to only occur very close to polymer entanglement points. Because the spiropyrans are likely placed near entanglement points of two different chains, instead within a single chain as previously demonstrated, it is hypothesized that this is what provides the substantial increase in sensitivity.

Overall, short-chain spiropyran crosslinker in polystyrene has demonstrated high sensitivity, with a colorimetric change that starts when it only has 5% deformation (5% strain).

For comparison, a mechanophoric compound comprising a synthetic polymer, SP-PS-N₃, was prepared and crosslinked with Compound 9 for the preparation of a mechanophoric matrix (spiropyran-functionalized polystyrene).

The preparation of this control spiropyran-functionalized polystyrene follows a preparation method which includes the incorporation of the mechanophoric compound during the polymerization process, in contrast to the post-processing which is performed in the newly developed method. Here, the mechanophoric compound serves as a co-monomer in the formation of a polystyrene-based backbone. A procedure for its preparation is as follows. A procedure for its preparation is as follows.

### Preparation of a spiropyran-functionalized polystyrene SP-PS

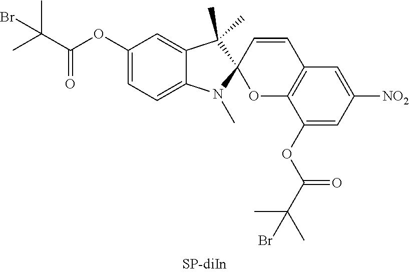

SP-diIn was prepared based on an established procedure [Davis et al., (2009) supra]. SP-PS-N₃ was prepared as described in FIG. 5B, and as follows:

### Synthesis of SP-PS-N₃

In a 10 mL Schlenk tube, copper (I) chloride (5 mg), PMDETA (12 μl), and toluene (0.5 ml) were mixed under argon. After 5 minutes, SP-diIn (25 mg) dissolved in toluene (1 mL) was added. Then, 4-azidomethylstyrene (0.15 mL) and styrene (0.9 mL) toluene were added to the reaction mixture. The reaction mixture was submitted to three freeze-pump-thaw cycles and then placed into an oil bath at 90° C. for 12 hours. The reaction was cooled producing a solid. The solid was slowly dissolved in CHCl₃ and passed through a pad of silica to remove the copper catalyst. The polymer solution was concentrated and precipitated into a large volume of diethyl ether. The slightly brown solid was dried under a vacuum. The chemical structure was verified by ¹H NMR (data not shown). Mw (THF-MALS GPC): 6.5 kDa, PDI: 1.3.

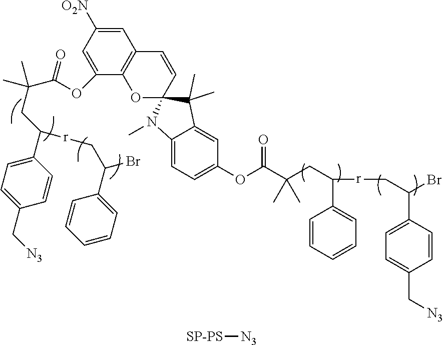

### Synthesis of SP-PS

SP-PS was formed following crosslinking of SP-PS-N₃ with the exemplary crosslinker Compound 9 (CL), following the cross-linking procedure as described hereinabove.

SP-PS was prepared via a standard random copolymer ATRP reaction. An example for such procedure is as follows: SP, vinyl styrene azide, vinyl styrene, Cu(I) (7.9 mg, 0.05436 mmol), and dry toluene (6 mL) were added into a Schlenk tube. The reaction was set under an Argon atmosphere for 1 hours to remove oxygen in the tube. PMDETA (34.7 μL, 0.1631 mmol) was then injected into the Schlenk tube with syringe, and the tube was placed in an oil bath for 24 hours at 90° C. The polymerization was manually stopped by exposing the reactant to air. The mixture was dissolved in THF and then passed through a column of neutral alumina to remove the residual copper catalyst. Finally, the clear solution was precipitated in hexane and the resulting solid cake was dried under vacuum at ambient temperature for 24 hours.

SP-PS formed a thin film while cross-linking.

This material was tested under tension (after plasticization as described above), and the results are shown in FIG. 5C. As B/G color ratios did not change significantly even at 0.6 (60%) strain, these data demonstrate that the norbornene-crosslinked spiropyran-functionalized polystyrene was incapable of activation in excess of 40% strain, at which the thin film ruptures. By shearing the film, as shown in FIG. 5D, fluorescence activation was observed exclusively at the rupture site.

These comparative data indicate that the method developed herein, which, inter alia, allows obtaining polymeric-based mechanophoric matrices via post-processing (post-polymerization) introduction of mechanophoric moieties is advantageous both in terms of the reaction conditions and in terms of the mechanophoric performance of the obtained mechanophoric matrix. Using a mechanophoric moiety with the same basic chemical structure resulted in much more sensitivity compared to the reference method, which suggests that the reason for the surprising sensitivity observations in the exemplary mechanophoric matrices arise from this method.

### Example 3

### Mechanophore-Conjugated Carbohydrates

Herein, alginate was used as an exemplary polysaccharide, and tetrazine was used as an exemplary moiety that can undergo a Click addition, to provide an exemplary natural polymer-based substrate, tetrazine-modified alginate (also being referred to herein as tetrazine alginate). An exemplary synthetic procedure for the preparation of tetrazine alginate is based on a known procedure [Desai et al., Biomaterials 2015, 50, 30] and is as follows.

### Synthesis of tetrazine alginate

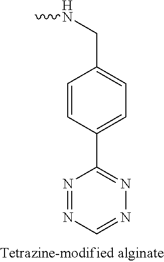

Sodium alginate (133 mg) was dissolved in 0.1 M MES, 0.3 M NaCl, pH 6.5 at 0.5% w/v buffer solution. EDC (0.6253 gram) and NHS (0.4635 gram) were added to the alginate buffer solution, (4-(1,2,4,5-tetrazin-3-yl)phenyl)methanamine hydrochloride (Tz; 25 mg) was added into the solution, and stirring took place at room temperature for 24 hours. Then, the reaction was quenched using hydroxylamine and dialyzed for 4 days using dialysis tubing against a decreasing salt gradient from 150 mM to 0 mM NaCl in deionized water (DW). The supernatant was freeze-dried after being centrifuged at 4000 rpm for 5 minutes to afford the tetrazine alginate (130 mg, 98%) as a pink fiber. The chemical structures was verified by ¹H NMR (D₂0), and the degree of tetrazine substitution of the available carboxylic acid groups of alginate was determined to be 2.82% by examining the absorption of the resulting tetrazine alginate (FIG. 6A) at 520 nm.

Molar attenuation coefficient (s) of the starting material ((4-(1,2,4,5-tetrazin-3-yl)phenyl)methanamine hydrochloride) was determined using Beer-Lambert law based on its absorption spectra (FIG. 6B).

Then, mono- and bis- norbornene spiropyran-functionalized tetrazine alginates were prepared according to an exemplary procedure, as follows.

### Preparation of mono- and bis- norbornene spiropyran-functionalized tetrazine alginate

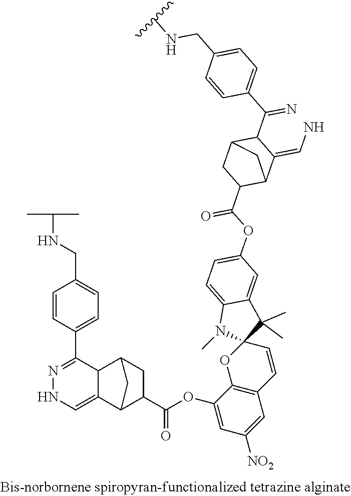 [SMILES: *NCc1ccc(C(=N)C2/C(=C\N)C3CC2CC3C(=O)Oc2ccc3c(c2)C(C)(C)[C@]2(C=Cc4cc([N+](=O)[O-])cc(OC(=O)C5CC6CC5C5=CNN=C(c7ccc(CNC(C)C)cc7)C56)c4O2)N3C)cc1]

(The norbonene is coupled to two alginate-tetrazine units in the polymeric matrix)

Tetrazine-modified alginate (30 mg) was fully dissolved in deionized water (2 mL), and a solution of bis- or mono- norbornene spiropyran (Compound 8 or Compound 7, respectively) in acetone (1 ml; 8.4 mM/mL) was added dropwise while stirring. The alginate solution was shaken vigorously during the mixing process. After 2 minutes, ALG-Mono and ALG-Bis were correspondingly produced. The chemical structures was verified by ¹H NMR (D₂O), and the degree of functionality as a result of the Click-conjugation was calculated to be 2.0 mol % per monomer (4 wt. %) based on the amount of the tetrazine consumed (data not shown).

The formed mono- and bis- norbornene spiropyran-functionalized tetrazine alginates were dissolved in an aqueous solution at 4° C. As shown in FIGS. 7A-B, while mono-norbornene spiropyran-functionalized tetrazine alginate remained liquid, bis-norbornene spiropyran-functionalized tetrazine alginate displayed enhanced gelation at the 20 same conditions.

Without being bound to any particular theory, it is assumed that bis-norbornene spiropyran-functionalized tetrazine alginate is crosslinked, and forms weak hydrogel. This further supports the hypothesis that the bis-norbornene spiropyran acts as a crosslinking agent.

Bending of thin alginate films was performed while examining colorimetric changes. For this purpose, a tetrazine-modified alginate thin film, a mono-norbornene spiropyran-functionalized tetrazine alginate film, and the exemplary bis-norbornene spiropyran-functionalized tetrazine alginate film were each folded between glass slides at varying angles, and fluorescence was examined (FIG. 8A) and quantified (FIGS. 8B-E).

As FIG. 8A shows, after folding the films 180°, the fluorescence at 575 nm was observed only for the bis-norbornene sample, indicating that the activation is solely due to the mechanical strain applied to the polymer film. As can be seen in FIG. 8B, folding the exemplary bis-norbornene spiropyran-functionalized tetrazine alginate film resulted in a clear peak in the visual spectrum (575 nm). In FIG. 8C, after folding over 135°, it is shown that spiropyran-functionalized alginate film shows colorimetric changes.

In order to examine the effect of impact on the exemplary bis-norbornene spiropyran-functionalized tetrazine alginate film, bis-norbornene spiropyran-functionalized tetrazine alginate solution (15 mg/mL) was injected into a round mold (internal diameter: 1.9 cm, height: 1 cm) and dried to a film. The formed film was then manually impacted using scissors, and was exposed to white light for 1 hour. Fluorescence analysis before and after impact was studied using a fluorescence microscope (Nikon ECLIPSE Ts2R) to follow the mechanochromic behavior and recovery behavior on alginate film. As shown in FIG. 9A-C, the exemplary bis-norbornene spiropyran-functionalized tetrazine alginate film remained impacted following exposure to a white light source.

To enhance the mechanical properties of the bis-norbornene spiropyran alginate film, the bis- and mono- norbornene spiropyran alginate films were prepared as composite films based on a known procedure [Sun et al., Nature 2012, 489, 133], as follows.

Preparation of bis- or mono- norbornene spiropyran-acrylamide alginate film: Bis- or mono- norbornene spiropyran-functionalized alginate (31 mg) was dissolved in 2 ml of a 12.4% acrylamide solution, followed by the addition of N,N-methylenebisacrylamide (0.0006 the weight of acrylamide) and ammonium persulfate (0.0004 of acrylamide). After vacuum degassing, N,N,N′,N′-tetramethylethylenediamin (0.0025 the weight of acrylamide) was added and mixed with 1.5% CaSO₄ (0.1328 of alginate), and injected into the mold.

The hydrogel was cured with 16 W power ultraviolet light (254 nm) at 50° C. for 45 minutes [Chen et al. Sci Adv. 2020, 6(20), eaaz5093]. After drying the film, the second polymerized network was formed by swelling the hydrogel with a 0.3 mL mixture solution of acrylamide, N,N-methylenebisacrylamide, and ammonium persulphate, as described herein. The spiropyran alginate hydrogel was subjected to degassing and cured under UV light. The hydrogel was dried in a chemical hood for one day, which resulted in a film.

To approximate the stresses and strains associated with the folding of the norbornene spiropyran-acrylamide alginate films, a finite element analysis (FEA) model was constructed using FEBio, as described in the Method section herein.

To determine the energy needed to activate by folding the exemplary spiropyran molecules, the volumetric distribution of the effective (von Mises) stresses and SED was plotted over the folding angle of the film (FIG. 8I).

As shown in FIG. 8J, both SED and effective stress increase while the film is folded on itself. FIG. 8J (top) shows the different steps of the film folding, where the apparition of a bulge at the basis of the film has very high folding angle values, suggesting the heterogeneous distribution of the effective stress in the film. This was further confirmed by the slice view on the Y-Z plane (x=0, center of the film; FIG. 8J, bottom), which shows that the maximal stresses are encountered at the base of the folding plane, at the contact point between the coverslip and the film, and finally at the most compressed and extend areas of the bulge.

FIG. 8G, which presents a plot showing stress-strain traction of the exemplary bis-norbornene spiropyran-tetrazine alginate hydrogel with a linear fit, shows the detection of a colorimetric change within the linear mechanical behavior of the polystyrene.

These data show that the method is not only surprisingly sensitive in detecting colorimetric changes upon application of force on mechanophoric matrices, but that it allows the detection of colorimetric change before the linear behavior ended, i.e., before material failure.

The exemplary bis-norbornene spiropyran-acrylamide alginate film was folded between glass slides at varying angles, as described herein. As can be seen in the photographs in FIGS. 10A-B, following folding (bending 180°), the film exhibited a clear change in color. Fluorescence intensity of bis-norbornene spiropyran-acrylamide alginate film was measured at 575 nm under various bending angles, and the results are presented in FIG. 11. A linear correlation is observed between the blending angle and average fluorescence.

### Example 4

### Mechanophore-Conjugated Natural Substrate

Herein, alpaca wool was used as an exemplary natural substrate. An exemplary procedure for the preparation of mono- or bis- norbornene spiropyran-functionalized wool is as follows.

Preparation of mono- or bis- norbornene spiropyran-functionalized wool: The wool fiber was pre-treated by (3-mercaptopropyl) methyldimethoxysilane (MPTES), based on a previously described procedure [Fan et al. Cellulose, 27, 493-510 (2020)], and as follows.

Wool fibers were extracted with acetone for 24 hours using a Soxhlet extractor to remove the dirt and surface lipids before use. Then, wool fibers were immersed in a detergent solution containing 15 gram/L NaOH and heated to 70° C. for 20 minutes to remove grease and destroyed disulfide bonds, which provided a favorable basis for the subsequent process. The fiber was washed with deionized water to mutualize then dry the fiber. 5% wt. MPTES was dissolved in an ethanol-deionized aqueous solution (v/v=4:1) by a shaker shake for 3 minutes. Then the fiber was immersed into an as—washed MPTES solution for 3 minutes and cured in a vacuum at 110° C. for 4.5 minutes. Pure water was used to wash the fiber. Then, bis/mono-norbornene spiropyran (Compound 8 or Compound 7, respectively) was dissolved in acetone at a ratio of 1:50, and BPO with 0.5% wt. was added as a photo-initiator. The MPTES-modified wool fiber was dipped into the mixture solution and irradiated under UV light for 1 hour. After completion of the reaction, the surface of the fiber was cleaned with standard deionized water. Drying under vacuum provided the final product.

The content of bis-spiropyran functional ratio on wool was evaluated by measuring the absorbance of the bis-norbornene spiropyran-functionalized wool sample, using Fourier transform infrared spectroscopy (FTIR), based on establishing a linear relationship between absorbance and the amount of bis-norbornene spiropyran in 200 mg of KBr pellet at a wavenumber of 2960 cm⁻¹. As can be seen in FIGS. 12A-B, the mass percentage of spiropyran in the bis-norbornene spiropyran-functionalized wool is 6.7% wt.

The amount of thiol groups in the wool during the functionalization process was determined as described herein, and the results are presented in FIGS. 13A-B. These data indicate MPTES treatment significantly increases the amount of thiol groups in the wool, and confirmed that most of the spiropyrans attached to the wool via MPTES, instead of via thiol group from the wool itself.

Tetraethyl orthosilicate (TEOS) was then used to conjugate the exemplary mechanophoric compound bis-norbornene spiropyran to alpaca wool fibers, by first conjugating TEOS to the wool fibers [Fan et al. Cellulose 2020, 27, 493-510], followed by the conjugation of the exemplary bis-norbornene spiropyran to the TEOS-wool. An exemplary procedure was performed as follows.

### Preparation of bis-norbornene spiropyran-conjugated TEOS-wool

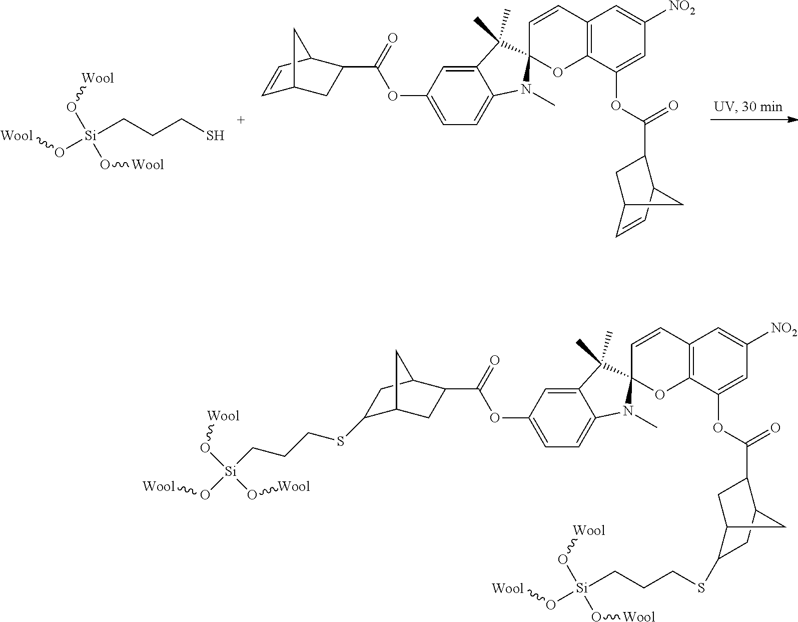

TEOS-wool and the exemplary bis-norbornene spiropyran (Compound 8) were mixed for 60 minutes under UV light (254 nm), cleaned with deionized water and dried under vacuum.

An exemplary protocol for functionalizing wool as an exemplary natural substrate with the exemplary mechanophoric compound, bis-norbornene spiropyran, is schematically illustrated in FIG. 14F.

Without being bound to any particular theory, this final functionalization step is believed to be carried out via a UV-thiol-ene Michael addition, wherein UV light activates the exemplary bis-norbornene spiropyran prior to a thiol-ene Michael addition with the exemplary MPTES-substituted wool fibers.

The bis-norbornene spiropyran-conjugated TEOS-wool (herein, also “activated wool” or “functionalized activated wool”) fibers were then tested by applying stress by manual rotation (twisting), as can be seen in FIG. 14A (the arrow points to the activated bis-norbornene spiropyran-conjugated TEOS-wool due to the shear forces along the twist axis). The bis-norbornene spiropyran-conjugated TEOS-wool was then compressed inside a one centimeter diameter cylindrical container (FIGS. 14B-C), and the strain was calculated from the change in the sample thickness as a result of compression at different measured pressures. At each pressure, the sample was removed to measure fluorescence at 575 nm. FIGS. 14A-D suggest that shear force and pressure can induce spiropyran activation to thus produce TEOS-wool colorimetric change.

The bis-norbornene spiropyran-conjugated TEOS-wool was then cut with scissors, as presented in FIG. 14E. These results show a color change following cutting the wool, and indicate that activation of the mechanophores can result from various mechanical actions, including cutting.

### Example 5

### Mechanophore-comprising Epoxy Resins

The use of the newly designed mechanophoric compounds for functionalizing epoxy resins is explored.

To this end, the exemplary mechanophoric compound, Compound 8, is used to functionalize epoxy resins such as typically used in paints.

An exemplary two-step procedure for the preparation of mechanophore-conjugated epoxy resins is presented in the below schemes, as follows.

Step 1: introducing to a mechanophoric compound according to the present embodiments, for example, Compound 8, a chemical group which reacts via Click reaction with an epoxide (e.g., an amine group such as TETA):

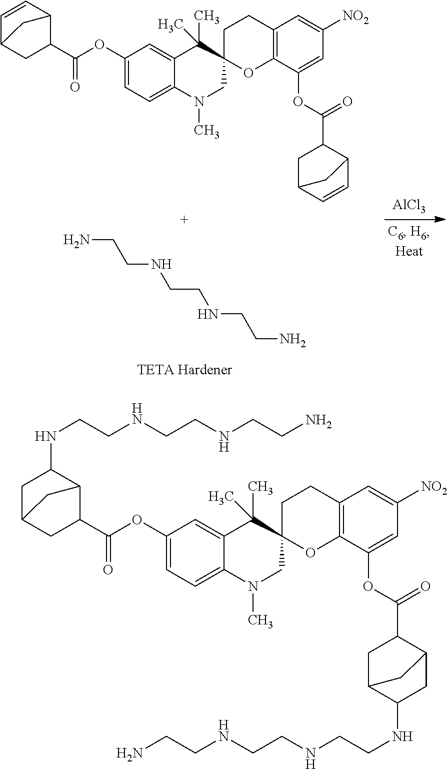

Step 2: coupling via Click chemistry (a reaction between epoxide and amine) the modified Compound 8 to epoxy groups of the epoxy resin (e.g., an epoxy resin such as DGEBA):

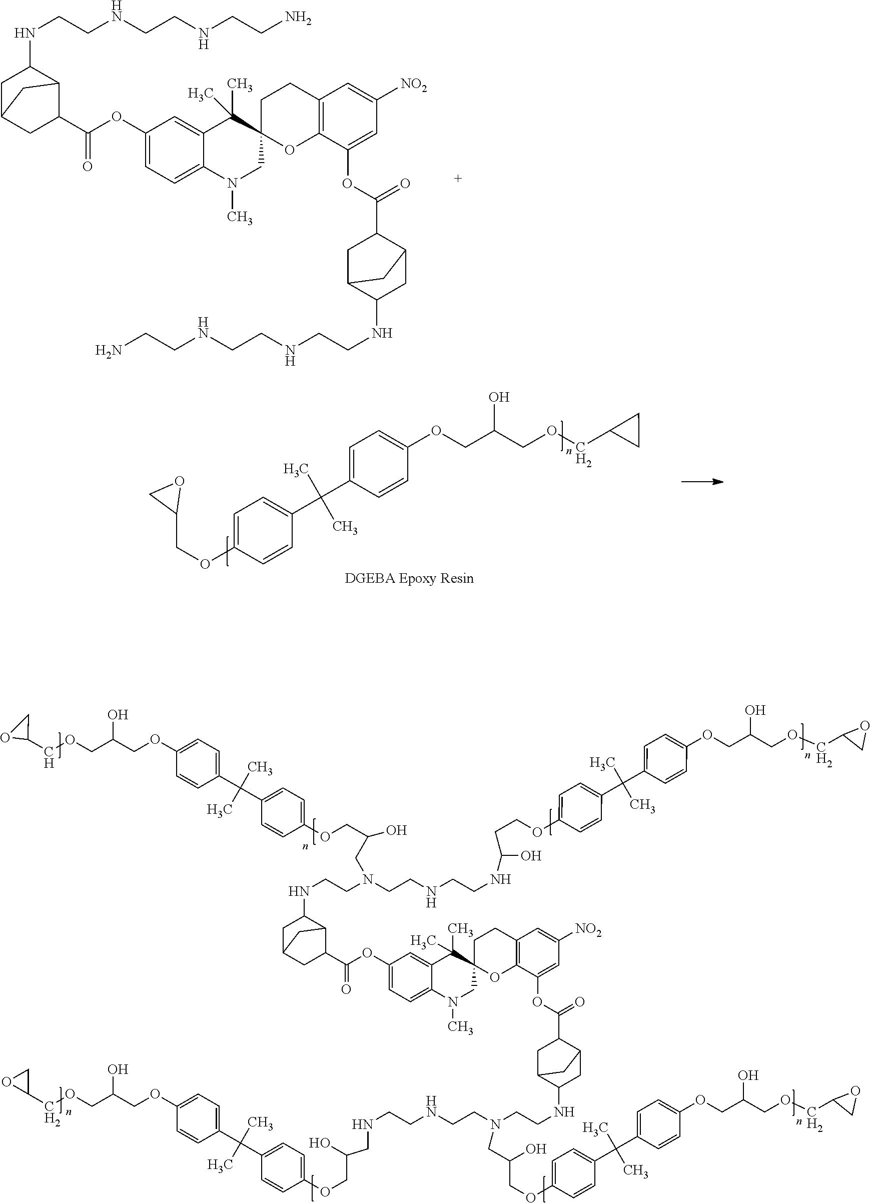

Epoxy resins are usable in a myriad of applications, including, for example, as pre-polymers for forming durable paint coating on various substrates, including metallic substrates. Incorporating mechanophores into epoxy-paints holds great potential, as it allows for the creation of thin, functional layers on a wide variety of articles, including, for example, metallic articles such as aluminum-containing articles.

### Example 6

### Mechanophore-Conjugated Metal Substrate

The use of the newly designed mechanophoric compounds for functionalizing metal-containing substrates was explored.

To this end, steel foils were sputter-coated with gold, and the gold was then annealed in an oven at about 700° C. Thiol groups were introduced to the gold-coated film by immersing it in an ethanolic solution of dithiothreitol (DTT) for 24 hours, as schematically illustrated in FIG. 15A. Photograph of the resulting gold film are also presented in FIG. 15A.

The film was then reacted with the exemplary bis-norbornene spiropyran (Compound 8), via an exemplary preparation procedure, as follows:

The gold plate was dipped into 0.05 M DTT (Dithiothreitol) ethanol solution overnight. Before introducing to the exemplary bis-norbornene spiropyran, the gold plate was dipped into 0.5 M DTT (Dithiothreitol) ethanol solution for 20 min. Then the plate was washed with a lot of water. Then, the gold plate was immersed in a solution with 3 mg/mL Compound 8 in ethanol with 0.06 mg/mL lithium phenyl-2,4,6-trimethyl benzoyl phosphinate photoinitiator. Exposure of the gold plate to 254 nm UV for 40 minutes provided the mechanophore-conjugated gold plate.

The surface contact angle was measured by placing a DI water droplet on the foil at different stages of the reaction process. The data was measured using a goniometer, and are presented in FIG. 15B.

As can be seen in FIG. 15B, a native gold surface is fairly hydrophobic, with a contact angle with the water droplet at about 96°. Following the thiol functionalization, a reduced contact angle (about 38°) was obtained, which indicates that the film became more hydrophilic, which can be explained by the presence of thiols.

After the film reacts with the exemplary bis-norbornene-spiropyran, which is hydrophobic, the surface of the film also becomes more hydrophobic (FIG. 15B, upper right image), thus supporting the successful functionalization of the thiol-substituted metal substrate. Upon UV-activation, as shown in FIG. 15B (bottom), a reduction in contact angle is observed, indicating the conversion of the spiropyran to the changed and more hydrophilic merocyanine form, which is visualized by the change in the surface wettability.

For comparison, functionalization of a gold-film with Compound 7 is performed.

IR and Raman spectroscopy are used to quantify and further analyze the spiropyran-functionalized surface of the gold film.

The newly prepared mechanophore-conjugated metallic surfaces may be useful as a novel and mechanical measurement tool. Such tool may utilize the ability of the spiropyran to become mechanically activated, as the change in the mechanophore could be translated into a change in the resistivity of the film, so it could potentially lead to something more sensitive than piezo-resistors because these are molecular level force sensors. Another macro-level use is the detection of damage in mechanically stressed parts using the mechanophore-conjugated metallic substrate.

These data further highlight the versatility of the method for preparing mechanophore-conjugated substrates, demonstrating that it can effectively utilize a variety of substrates functionalized with reactive groups such as thiols.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of preparing a mechanophoric matrix, the method comprising contacting a substrate that features a plurality of a first reactive group with a mechanophoric compound that features at least one of a second reactive group, wherein said first and second reactive groups are complementary clickable groups that are capable of undergoing a click reaction with one another to thereby form a covalent Click bond therebetween, thereby preparing the mechanophoric matrix.

2. The method of claim 1, wherein said second reactive group is or comprises a strained cycloalkyl or a strained unsaturated cycloalkyl.

3. The method of claim 2, wherein said second reactive group is or comprises a norbornene.

4. The method of claim 1, wherein said substrate is or comprises a polymeric material.

5. The method of claim 1, wherein said substrate is or comprises a metallic material.

6. The method of claim 1, further comprising, prior to said contacting, generating at least one, or a plurality, of said first reactive group in and/or on said substrate.

7. The method of claim 1, wherein said mechanophoric compound is represented by Formula I: 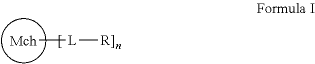
wherein:
Mch is a force-responsive moiety;
L is a linking group or absent;
R is said second reactive group; and
n is an integer greater than 1.

8. The method of claim 7, wherein Mch is or comprises a spiropyran.

9. The method of claim 8, wherein said mechanophoric compound is represented by Formula II: 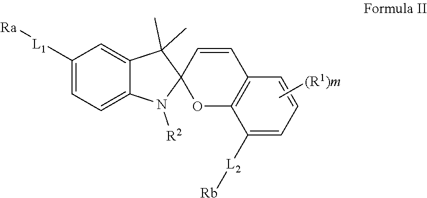
wherein:
Ra and Rb are each independently said second reactive group;
L₁ and L₂ are each independently said linking group or is absent;
R¹ is a substituent selected from alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, carbamate, thiocarbamate, amide, carboxylate, sulfonamide, guanyl, guanidinyl, hydrazine, hydrazide, thiohydrazide, and amine;
m is 0, 1, 2 or 3, representing the number of the R¹ substituent(s); and
R² is selected from hydrogen, alkyl, cycloalkyl, and aryl.

10. The method of claim 9, wherein at least one, or each, of Ra and Rb is or comprises a strained cycloalkyl or a strained unsaturated cycloalkyl.

11. The method of claim 10, wherein at least one, or each, of Ra and Rb is or comprises a norbornene.

12. The method of claim 1, wherein said mechanophoric compound is:

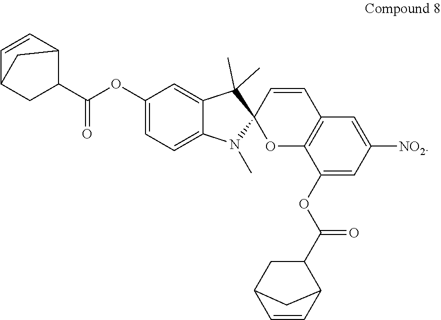 [SMILES: CN1c2ccc(OC(=O)C3CC4C=CC3C4)cc2C(C)(C)[C@]12C=Cc1cc([N+](=O)[O-])cc(OC(=O)C3CC4C=CC3C4)c1O2]

13. A mechanophoric matrix obtainable by the method of claim 1.

14. A mechanophoric compound represented by Formula I*: 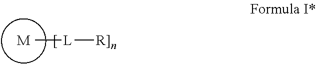
wherein:
Mch is a force-responsive moiety;
L is a linking group or absent;
R is a clickable group which is a reactive group capable of participating in a Click reaction by forming a covalent bond with a complementary clickable group; and
n is a positive integer or an integer greater than 1.

15. The mechanophoric compound of claim 14, wherein Mch is or comprises a spiropyran.

16. The mechanophoric compound of claim 15, represented by Formula II*: 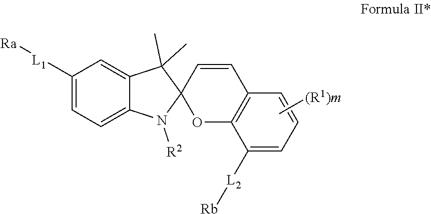
wherein:
Ra and Rb are each independently selected from a substituent and said clickable group, or is absent, wherein at least one of Ra and Rb is said clickable group, and wherein said substituent, if present, is selected from alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, carbamate, thiocarbamate, amide, carboxylate, sulfonamide, guanyl, guanidinyl, hydrazine, hydrazide, thiohydrazide, and amine;
L₁ and L₂ are each independently said linking group or is absent;
R¹ is a substituent selected from alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, urea, thiourea, carbamate, thiocarbamate, amide, carboxylate, sulfonamide, guanyl, guanidinyl, hydrazine, hydrazide, thiohydrazide, and amine;
m is 0, 1, 2 or 3, representing the number of the R¹ substituent; and
R² is selected from hydrogen, alkyl, cycloalkyl, and aryl.

17. The mechanophoric compound of claim 16, wherein said clickable group is or comprises a strained cycloalkyl or a strained unsaturated cycloalkyl.

18. The mechanophoric compound of claim 17, wherein each of Ra and Rb independently is or comprises a norbornene.

19. A mechanophoric matrix (a mechanophore-functionalized matrix), comprising a substrate having at least one, or a plurality of, a mechanophoric moiety derived from the mechanophoric compound of claim 14 covalently coupled to the substrate, wherein each of said at least one, or plurality of, said mechanophoric moiety is coupled to the substrate via a Click bond formed by a Click reaction between a second reactive group which is said clickable group of the mechanophoric compound and a complementary first reactive group which is a clickable group present or generated in said substrate.

20. The mechanophoric matrix of claim 19, capable of exhibiting a force-induced colorimetric change following deformation of at least 1%, or at least 5%, or at least 10% and/or following deformation of less than 200%.

21. The mechanophoric matrix of claim 19, wherein upon application of force, the mechanophoric matrix features an average B/G color ratio higher by at least 10% than an average B/G color ratio of an intact mechanophoric matrix, when determined by converting an image of the mechanophoric matrix and an image of the intact mechanophoric matrix to RGB signals and calculating an average B/G color ratio therefrom.

22. The mechanophoric matrix of claim 21, capable of exhibiting a force-induced colorimetric change following force application in an amount of at least 300 Pa, or at least 400 Pa, or at least 450 Pa, or at least 500 Pa, or at least 550 Pa.

23. An article-of-manufacturing comprising the mechanophoric matrix of claim 19.

24. A method for determining a presence and/or a level of a force-induced damage in the mechanophoric matrix of claim 19 or in an article-of-manufacturing comprising said mechanophoric matrix, the method comprising determining a colorimetric change in said matrix or said article-of-manufacturing, said colorimetric change being indicative of the presence and/or the level of the force-induced damage in the matrix or article-of-manufacturing.

25. The method of claim 24, wherein said force-induced damage is following deformation of at least 1%, or at least 5%, or at least 10% and/or lower than 200% or lower than 100%.

26. The method of claim 24, wherein said force-induced damage is following force application in an amount of at least 300 Pa, or at least 400 Pa, or at least 450 Pa, or at least 500 Pa, or at least 550 Pa.

27. The method of claim 24, wherein said colorimetric change comprises a change in an average B/G color ratio, when determined by converting an image of the mechanophoric matrix and an image of the intact mechanophoric matrix to RGB signals and calculating an average B/G color ratio therefrom.

28. A mechanophoric matrix comprising a metallic substrate having at least one mechanophoric moiety covalently attached thereto.

29. The mechanophoric matrix of claim 28, wherein said mechanophoric moiety is derived from a mechanophoric compound that features at least one clickable group and is covalently coupled to said metallic substrate via a Click bond formed by a Click reaction between said at least one clickable group of said mechanophoric moiety and a complementary clickable group generated in and/or on said metallic substrate.

30. The mechanophoric matrix of claim 29, wherein said metallic substrate comprises a metallic film featuring said complementary clickable group.
